(19) United States
(12) Patent Application Publication
(10) Pub. No.: US 2025/0377343 A1
(43) Pub. Date: Dec. 11, 2025

(54) ADVANCED CALIBRATION SYSTEMS AND METHODS FOR ENHANCED ENVIRONMENTAL AND AIR QUALITY MONITORING

(71) Applicant: CLARITY MOVEMENT CO., Berkeley, CA (US)

(72) Inventors: Paolo Micalizzi, Milan (IT); Baljot Singh, Daly City, CA (US); John Kelly Kodros, Fort Collins, CO (US); Levi George Stanton, New York, NY (US); Kenneth Neal McGary, San Francisco, CA (US)

(73) Assignee: CLARITY MOVEMENT CO., Berkeley, CA (US)

(21) Appl. No.: 19/234,220

(22) Filed: Jun. 10, 2025

Related U.S. Application Data
(60) Provisional application No. 63/658,395, filed on Jun. 10, 2024.

(51) Int. Cl. G01N 33/00

(52) U.S. Cl. CPC G01N 33/0008; G01N 33/0034

(57) ABSTRACT

Systems, methods, and computer-readable media for calibrating air quality sensors are provided. A sensor node includes a sensor node printed circuit board, a sensor module, and a communication module. The sensor node printed circuit board manages power of the sensor node circuitry, the sensor module, and the communication module such that power is provided from a primary power supply supplemented by a secondary power supply. The sensor module includes a plurality of air quality sensors to measure the concentration of air pollutants. The sensor module may be replaceable. The communication module may communicate air quality measurements to and receive configurations from a data management platform, which may perform processes to improve the accuracy of the air quality measurements.

### CROSS-REFERENCE TO RELATED APPLICATION(S)

This application claims the benefit of prior filed U.S. Provisional Patent Application No. 63/658,395, filed Jun. 10, 2024, which is hereby incorporated by reference herein in its entirety.

### COPYRIGHT NOTICE

At least a portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### TECHNICAL FIELD

This disclosure relates to environmental monitoring, specifically focusing on advanced systems and methods for calibrating air quality sensors. This includes innovative techniques for calibrating particulate matter sensors, electrochemical cell sensors measuring gaseous pollutants, and aethalometers measuring black carbon. These systems and methods address the existing challenges in calibration accuracy, sensor integration, and data usability.

### BACKGROUND OF THE DISCLOSURE

Sensor calibration must be improved to enable more accurate systems.

### SUMMARY OF THE DISCLOSURE

This document describes systems, methods, and computer-readable media for calibrating air quality sensors.

For example, a method for calibrating air quality sensors is provided.

As another example, a system for calibrating air quality sensors is provided.

As yet another example, a non-transitory computer-readable storage medium storing at least one program is provided, the at least one program including instructions, which, when executed by at least one processor of an electronic subsystem, cause the at least one processor to calibrate air quality sensors.

One or more improved systems, methods, and/or apparatuses acquire air quality measurements within a given region with high spatiotemporal resolution and high measurement accuracy. Hyperlocal monitoring of air quality within a given region may occur through the deployment of a dense network of environmental sensor nodes. Systems, methods, and/or apparatuses are further provided to ensure and enhance the accuracy of the measurements of said sensor nodes, and that enable the quick and scalable deployment of a dense network of said sensor nodes.

A compact sensor apparatus is disclosed, which includes a power module configured to supply a reliable power supply from a primary power source supplemented by a secondary power source. The apparatus further includes a sensor module configured to monitor a gas, such as ambient air, for one or more characteristics. The apparatus includes a communication module configured to establish a wireless communication channel over a network with a host. The apparatus further includes a controller configured to manage the sensor module and to send measurement data to the host by way of the wireless communication channel. The apparatus includes a printed circuit board configured to interconnect the power module, reliable power supply, controller, and communication module. Finally, the apparatus includes an enclosure configured to house the printed circuit board, power module, sensor module, communication module, and controller.

A system is disclosed including an interchangeable sensor module configured to monitor an air sample for one or more characteristics. The system further includes an enclosure including the interchangeable sensor module, a power module configured to supply power, a communication module configured to establish a wireless communication channel over a network with a host, a controller configured to manage the interchangeable sensor module and to send measurement data to the host by way of the wireless communication channel. Finally, the system includes a universal mount configured to mount the enclosure in a plurality of mounting configurations.

A method is disclosed, which includes placing a first sensor node near a reference monitor within a region. The method next includes placing a plurality of sensor nodes at various locations within the region. The method further includes gathering measurement data from the first sensor node, the reference monitor, and the plurality of sensor nodes. Then the method includes determining a calibration profile for each of the first sensor node and the plurality of sensor nodes based on measurement data from the reference monitor. Finally, the method includes applying the calibration profile for each of the first sensor node and the plurality of sensor nodes to measurement data from each of the first sensor node and the plurality of sensor nodes to obtain calibrated measurement data for each of the sensor nodes.

In some embodiments, a method is disclosed that may include accessing collocation data for each one of a plurality of collocations, wherein: the accessed collocation data for each collocation of the plurality of collocations includes: sensor data collected from a sensor of the collocation over a collocation period of time of the collocation; and reference monitor data collected from a reference monitor of the collocation over the collocation period of time of the collocation; and, for each collocation of the plurality of collocations, the sensor of the collocation was collocated with the reference monitor of the collocation during the collocation period of time of the collocation at a location of the collocation; combining the accessed collocation data from each one the plurality of collocations into global collocation data; developing a global calibration on the global collocation data; and configuring a calibration regulator for a sensor of interest (“SOI”) to apply the developed global calibration.

In some embodiments, a non-transitory computer-readable storage medium is disclosed that may store at least one program, the at least one program including instructions, which, when executed by at least one processor of an electronic subsystem, cause the at least one processor to: access collocation data for each one of a plurality of collocations, wherein: the accessed collocation data for each collocation of the plurality of collocations may include: sensor data collected from a sensor of the collocation over a collocation period of time of the collocation; and reference monitor data collected from a reference monitor of the collocation over the collocation period of time of the collocation; and, for each collocation of the plurality of collocations, the sensor of the collocation was collocated with the reference monitor of the collocation during the collocation period of time of the collocation at a location of the collocation; combine the accessed collocation data from each one the plurality of collocations into global collocation data; develop a global calibration on the global collocation data; and configure a calibration regulator for a sensor of interest (“SOI”) to apply the developed global calibration.

In some embodiments, a system is provided that may include: a memory component; a communications component; and a processor component configured to: access collocation data for each one of a plurality of collocations using the communications component, wherein: the accessed collocation data for each collocation of the plurality of collocations may include: sensor data collected from a sensor of the collocation over a collocation period of time of the collocation; and reference monitor data collected from a reference monitor of the collocation over the collocation period of time of the collocation; and for each collocation of the plurality of collocations, the sensor of the collocation was collocated with the reference monitor of the collocation during the collocation period of time of the collocation at a location of the collocation; combine the accessed collocation data from each one the plurality of collocations into global collocation data; develop a global calibration on the global collocation data; and configure, in the memory component, a calibration regulator for a sensor of interest (“SOI”) to apply the developed global calibration.

This Summary is provided to summarize some example embodiments, so as to provide a basic understanding of some aspects of the subject matter described in this document. Accordingly, it will be appreciated that the features described in this Summary are only examples and should not be construed to narrow the scope or spirit of the subject matter described herein in any way. Unless otherwise stated, features described in the context of one example may be combined or used with features described in the context of one or more other examples. Other features, aspects, and advantages of the subject matter described herein will become apparent from the following Detailed Description, Figures, and Claims.

### BRIEF DESCRIPTION OF THE DRAWINGS

The discussion below makes reference to the following drawings, in which like reference characters may refer to like parts throughout, and in which:

FIG. 1 illustrates a schematic view of an embodiment of a sensor node, according to some embodiments of the disclosure;

FIG. 2 illustrates a diagram of an embodiment of a printed circuit board, according to some embodiments of the disclosure;

FIG. 3 illustrates an exploded view of an embodiment of a sensor node, according to some embodiments of the disclosure;

FIG. 4 illustrates a top view of a sensor node, according to some embodiments of the disclosure;

FIG. 5 illustrates a dual sensor module configuration, according to some embodiments of the disclosure;

FIG. 6 illustrates an exploded view of an embodiment of an interchangeable sensor module, according to some embodiments of the disclosure;

FIG. 7 illustrates a diagram of an embodiment of a sensor module printed circuit board, according to some embodiments of the disclosure;

FIG. 8 illustrates a cross-sectional view of a sensor module, according to some embodiments of the disclosure;

FIG. 9 illustrates an embodiment of a hyperlocal air quality monitoring system 900, according to some embodiments of the disclosure;

FIG. 10 illustrates a routine, according to some embodiments of the disclosure;

FIG. 11 illustrates an embodiment of a calibration method, according to some embodiments of the disclosure;

FIG. 12 illustrates an embodiment of a sensor module measurement method, according to some embodiments of the disclosure;

FIG. 13 is an example block diagram of a computing device, according to some embodiments of the disclosure; and

FIGS. 14-23 illustrate flowcharts of exemplary processes for calibrating sensors, according to some embodiments of the disclosure.

### DETAILED DESCRIPTION OF THE DISCLOSURE

Systems, methods, and computer-readable media for calibrating air quality sensors are provided.

Air pollution is a leading cause of premature deaths worldwide and represents a high cost in terms of welfare spending. Therefore, governments and other organizations are mandated to monitor air quality and reduce exposure of people to air pollution. Conventionally, air quality within a given region, for example in a city, is monitored using expensive monitoring equipment with bulky size, high cost, and high maintenance requirements. Due to budget and space constraints, some monitoring systems and methods may only be deployed at sparse locations within the region, which limits the ability of acquiring air quality information with high spatiotemporal resolution. The limitations in air quality information may hinder the ability to take effective actions for reducing air pollution. To address the need for air quality information with higher spatiotemporal resolution, the deployment of dense networks including numerous low-cost, internet connected environmental sensors (e.g., sensor nodes) is attractive. However, the accuracy of sensor nodes may be lower than that of the conventional monitoring equipment (e.g., monitors), which causes concerns regarding the accuracy of the information they acquire. Therefore, there is a need to provide systems and methods for hyperlocal monitoring of air quality within a given region with high spatiotemporal resolution and high measurement accuracy. Furthermore, increasing the number of monitoring sites could result in an increase of device deployment and maintenance cost. Thus, there is a need for systems that can be efficiently deployed and maintained.

A compact sensor apparatus in the form of a sensor node is disclosed herein. The sensor node may be considered compact in that it may be between 50 mm and 200 mm in length, between 40 mm and 100 mm in width, and between 40 mm and 100 mm in depth, although any other suitable dimensions and/or ranges thereof may be utilized. In some embodiments, the sensor node may weigh less than 1500 grams. The sensor node may include a printed circuit board, a communication module, and a sensor module that may be enclosed in a weatherproof enclosure. “Sensor node” may refer to a device or apparatus configured as recited in one or more of the claims or embodiments of this disclosure. In particular, a sensor node may be a lightweight, compact device configured to include its own power source(s) and to communicate measurement data over a wireless communication channel to a host. “Sensor module” may refer to a device, component, circuit, system, chip, or circuitry configured to detect and/or measure one or more characteristics of matter. A sensor module, in one embodiment, may be configured to detect and/or measure levels of certain elements and/or particulates in a gas or a gas mixture, including, but not limited to, air. “Gas” may refer to any substance or combination of substances in a gaseous state of matter. Examples of a gas include, but are not limited to, ambient air, driven air, a gas of a single element like hydrogen, nitrogen, or the like, or a gas of a compound such as chlorine, nitrous oxide, or the like. Furthermore, as used herein gas may refer to substances that are a pure composition of one or more elements as well as substances that include contaminants, both gaseous contaminants and particulate contaminants. The modularity of the sensor node may enable it to be configured differently depending on deployment scenarios to ensure scalable deployment of a dense sensor network in a region where air quality is measured.

The sensor node printed circuit board may include a controller that collects data from the sensor module and sends it to a data management platform using the communication module, and a power module that manages power delivery, battery charging, and power monitoring. The communication module may interface with the sensor node printed circuit board via a mini peripheral component interconnect (“PCI”) or PCIe interface and may use any wireless technology including but not limited to WiFi, long-term evolution (“LTE”), long range (“LoRa”™), and narrowband internet of things (“NB-IoT”) to send data from the sensor node to the data management platform.

The sensor module may interface with the sensor node printed circuit board via wire to board connectors. The sensor module may include a plurality of air quality sensors, which may measure the concentration of air pollutants. The sensor module may include at least one air quality sensor with an active sampling mechanism, such as a fan or a blower. The structure of the sensor module and the placement of the air quality sensors within the sensor module may be configured in such a way that the active sampling mechanism of one of the air quality sensors is used to expose all air quality sensors in the sensor module to samples of air from the ambient environment.

The sensor module may store instructions to measure the concentration of several air pollutants through several air quality sensors. The sensor node may be further configured to acquire air quality measurements, communicate air quality measurements to a data management platform, and receive configurations from a data management platform. The communication between sensor node and data management platform may be through a data network that is configured in a secure way and with low data overhead.

In further embodiments, a solar panel may be mounted to the front of the sensor node through a gimbal fastener. The gimbal fastener may be oriented to maximize the exposure of the solar panel to direct sunlight. In certain embodiments, a user or technician may orient the solar panel in the field by adjusting the gimbal fastener. The solar panel may be coupled to the power module within the sensor node through a connector.

Hyperlocal air quality monitoring may include multiple sensor nodes deployed in a region. The system may include sensor nodes that are deployed in close proximity to highly accurate monitors found in the region. The system may include a data management platform that is configured to receive and process air quality measurements acquired by the sensor nodes and the monitors, identify co-location pairs as pairs of sensor nodes and monitors that are in close proximity to each other, create calibration profiles by calibrating the sensor nodes against the co-located monitors, correct measurements from sensor nodes according to the calibration profiles, store information in storage media, and/or make information stored in storage media available to data consumers through data interfaces.

The system may include a method to identify co-location pairs as pairs of sensor nodes and monitors that are deployed in close proximity to each other, and to calculate calibration profiles by calibrating the sensor nodes against the co-located monitors. Other sensor nodes may have their measurements corrected by applying a calibration profile.

This disclosure introduces a comprehensive method for calibrating environmental and air quality sensors, addressing the limitations of low-cost sensors in accurately measuring various pollutants, including, but not limited to, particulate matter (“PM”) (e.g., PM_{2.5} (e.g., particles with aerodynamic diameter≤2.5 μm), PM₁₀ (e.g., particles with aerodynamic diameter≤10 μm), total suspended particulate (“TSP”) (e.g., particles with aerodynamic diameter≤100 μm), ultrafine particles (e.g., particles with aerodynamic diameter≤0.1 μm), and/or the like), gas-phase pollutants (e.g., nitrogen dioxide (“NO₂”), nitric oxide (“NO”), carbon monoxide (“CO”), ozone (“O₃”), hydrogen sulfide (“H₂S”), sulfur dioxide (“SO₂”), ammonia (“NH₃”), and/or the like), black carbon (“BC”), and/or the like. Black carbon may be essentially the light-absorbing fraction of PM (often called soot). In this disclosure, it may sometimes be treated separately because it may be measured by aethalometers rather than by PM sensors and/or gas sensors. Other PM subtypes, such as elemental carbon (“EC”) measured by thermo-optical methods or organic carbon (“OC”) measured by thermal desorption) may in principle be grouped with BC. But in practice for aethalometers, “black carbon” may be unique. It may not fit under “gas-phase,” and it may not just be “PM mass,” since it may be specifically the light-absorbing component. So in the context of this disclosure, BC may stand alone as its own monitored species. These sensors, which may include, but are not limited to, optical particle counters (“OPCs”), nephelometers, electrochemical cells (“ECS”), and aethalometers, may often suffer from environmental interferences, sensor-to-sensor variability, cross-sensitivities, and/or poor alignment with reference-grade monitors. This disclosure proposes advanced calibration strategies to correct for these inaccuracies using physics-based and machine learning models, both globally and locally.

For particulate matter, this disclosure notes that PM sensors may be configured to count particles across size bins rather than directly measuring PM mass. Conversion to mass may require one or more assumptions about particle properties, which can vary regionally. To correct for this, one or more methods of this disclosure may include training a model (e.g., a multiple linear regression model) on collocation data collected worldwide (e.g., to correct for the error that may be caused by using a single factory-calibration derived conversion (e.g., count to mass) across all regions (e.g., because each region's aerosol composition, density, and size distribution can differ, a PM sensor mass estimate will likely be biased if those assumptions do not match reality), and a global model may aim to “correct for” that bias (e.g., to reduce the error between the PM sensor's mass estimate and the true PM mass (e.g., as may be measured by reference monitors) across all regions)). “Regionally” may refer to a specific geographic area sharing similar emission sources, meteorology, and/or aerosol composition. For example, a desert region may have primarily mineral dust (e.g., low density, large size), whereas an urban industrial region may have more combustion-derived soot (e.g., higher density, smaller size). As PM sensors might estimate mass using assumed density and refractive index, those assumed values may be valid in one region but not another. Thus, “assumptions” can be tuned by region. “Worldwide” may refer to aggregating data from many such distinct regions (e.g., essentially a global dataset that may span all major emission sources, climates, and aerosol composition types). There may be no fixed number of “regions” for “worldwide”. Instead, it may mean “as many distinct geographic/climactic/source domains as possible” to capture global variability. In practice, one might divide the globe into a few dozen climatological or emission-based zones (e.g., North America urban, East Asia industrial, Sub-Saharan dust, European mixed, etc.). Those may become the “regions” whose data may feed into a global model. Therefore, “regionally” may be a single, localized domain with its own typical environment and particle characteristics (and therefore its own calibration assumptions), while “worldwide” may be a union of data from many of those domains to train a universally applicable calibration. It is to be understood that the terms “collocate” and “co-locate” as used herein may each denote deploying a sensor node side by side with a reference instrument so that they may share the same or substantially the same air mass. This global PM calibration may reduce error by applying generalized correction factors, where the error may be the difference between the raw sensor output (e.g., mass estimate from a PM sensor) and the true pollutant concentration measured by a reference monitor, where such an error may be caused by any suitable source(s), including, but not limited to, assumed particle properties (e.g., density, refractive index) that do not match local aerosol, environmental interferences (e.g., high humidity causes hygroscopic growth, altering scattering), sensor nonlinearity or saturation at high concentrations, instrument-to-instrument variability (e.g., manufacturing tolerances), cross-sensitivities, decreased detection ability of particles above or below a certain size, and/or the like. In practice, once a sensor is deployed, its raw readings (e.g., raw PM_{2.5} mass concentration [μg/m³], raw PM_{2.5} number concentration [#/cm³], etc.) plus measured environmental parameters (e.g., T, RH, etc.) and possibly derived features (e.g., as described herein) may be fed into a calibration model (e.g., a model that may run on the device's microcontroller in firmware, or on a cloud server running a data pipeline to which the device may upload the data wirelessly, etc. and/or that may otherwise be utilized for providing a calibration regulator for the device (e.g., for a sensor component of interest of a sensor node and/or sensor module, etc.)). The model may then be configured to output a corrected concentration (e.g., calibrated PM_{2.5} mass concentration [μg/m³]). That corrected value may be what the end user sees as one of the outputs of the sensor (e.g., if the calibration is running onboard, the end user might see the calibrated measurement as one of the serial outputs of the sensor), in dashboards, application programming interface (“API”) endpoints, databases, and/or the like and it may align more closely with reference-grade instruments. For local precision, additional collocation-based calibrations can be conducted at deployment sites, thereby tailoring calibration to specific environmental and pollutant profiles.

For gas-phase pollutants (e.g., NO₂), sensors may be prone to cross-sensitivities and/or environmental shifts. To address this, one or more methods of this disclosure may include using one or more models (e.g., one or more ensemble machine learning models (e.g., a Light Gradient-Boosting Machine (“LightGBM”) or any other suitable distributed gradient-boosting framework for machine learning)) that may be trained on one or more global datasets. In both such cases, the “collocation data collected worldwide” may refer to a global dataset that may aggregate collocation measurements from many sites around the world that may cover diverse climates, pollutant mixtures, and operating conditions. That dataset may be used to train a global calibration model (e.g., a LightGBM model for NO₂, a multiple linear regression model for PM_{2.5}, etc.). However, developing a global calibration (e.g., a global calibration model with features, hyperparameters, coefficients, etc.) for different pollutants may include dedicated collocation data for each target. Although that global dataset may include some of the same sites, it may be filtered per pollutant (e.g., only those collocations where a reference NO₂ analyzer was present). In other words, the broad “worldwide” dataset may feed multiple global calibration efforts (e.g., PM, NO₂, CO, etc.), but each pollutant's model may be trained on the subset of collocations relevant to that pollutant. Thus, the global PM collocation dataset and the global NO₂ collocation dataset may overlap geographically but may differ in which sensors, references, and/or quality filters may be applied. These models may be configured to correct sensor output by accounting for any suitable variables, including, but not limited to, temperature, humidity, barometric pressure, wind speed, wind direction, particulate composition proxies, time of day, solar radiation, traffic and/or road proximity, population density or land use index, altitude, and/or the like. Additionally or alternatively, one or more methods of this disclosure may be configured to support site-specific collocation studies to fine-tune sensor accuracy in local conditions (e.g., providing the procedures, data pipelines, model templates, and/or the like so that a user may carry out any suitable processes, including, but not limited to, deploying sensor beside reference (e.g., place a low-cost sensor immediately adjacent to a reference-grade instrument at the intended monitoring location), collecting collocation data (e.g., record raw sensor outputs along with environmental measurements and the reference monitor's true pollutant readings over several weeks), training collocation-based calibration (e.g., fit a custom calibration model to the collected collocation data), validating and finalizing (e.g., evaluate performance metrics (e.g., R², RMSE, etc.) to ensure the local calibration may improve accuracy beyond the global calibration (e.g., if acceptable, freeze the custom model parameters)), applying to deployed sensor (e.g., load a custom collocation-based calibration into the sensor's firmware or cloud pipeline, where, from that point on, each measurement may be first run through the custom collocation-based calibration, yielding a finely tuned output tailored to that deployment), and/or the like. By following these processes, a sensor can be “fine-tuned” to local conditions, thereby reducing bias and improving precision in that particular region, achieving better results compared to applying a global calibration.

Black carbon may be measured by aethalometers, which may be configured to infer concentrations from light attenuation through filters. These instruments can suffer baseline shifts due to rapid temperature changes, especially in outdoor deployments. One or more methods of this disclosure may include introducing a calibration method that characterizes each monitor's sensitivity to temperature ramps during production and applies regression-based correction, either in firmware or via cloud processing, to maintain data accuracy over time. For example, a process may include characterizing an aethalometer (e.g., in the factory, by running controlled temperature ramps under clean-air (e.g., HEPA-filtered) conditions) and, then, from that characterization, deriving regression coefficients (e.g., slope and intercept) that may relate the filter's baseline signal to the internal temperature's rate of change and, then, those slope/intercept values may be used to constitute the calibration coefficients to a simple linear calibration model that may predict baseline shift as a function of dT/dt and, then, in the field, as the aethalometer measures BC and logs temperature changes, applying that regression in real time (e.g., on the device or on the cloud) to subtract out the bias due to fast temperature changes. Thus, there may be a calibration (e.g., the temperature-rate regression), which may be applied to every raw BC measurement (e.g., to correct for baseline drift).

Calibration models may be trained using data from collocated sensors and reference monitors, capturing true pollutant concentrations across varied environments. These models may be configured to ingest raw sensor data along with features derived from environmental measurements and mathematical transformations. Environmental measurements (e.g., raw inputs) may include inputs that may be obtained by integrating the corresponding sensors into the same node and/or by pulling data from a local weather station via API, such as inputs including, but not limited to, temperature (“T”) (e.g., from an adjacent temperature sensor), relative humidity (“RH”) (e.g., from an adjacent relative humidity sensor), barometric pressure (“P”) (e.g., from an adjacent barometric sensor), auxiliary pollutant concentrations (e.g., raw O₃, NO, NO₂, CO, or CO₂ readings from adjacent sensors), wind speed and wind direction (e.g., from an adjacent anemometer or weather API data), time of day/timestamp (e.g., automatically logged with each sample), and/or the like. Mathematical transformations (e.g., derived features that may capture non-linearities, temporal dynamics, and/or event-driven anomalies in the raw data, including, but not limited to, temperature polynomial baseline (e.g., by T_{baseline}=c₁(T²−25²)+c₂(T−25)+c₃ (e.g., captures non-linear shifts in the sensor baseline as temperature deviates from 25° C. (e.g., many gas sensors baselines may exhibit a quadratic-like drift with T))), time-dependent RH baseline (e.g., exponential filter (e.g., RH_{baseline}t=ΔRH×δRhConst+R_{baseline}t−1×exp(—Δt/τ) (e.g., models baseline shifts caused by sudden humidity changes while attenuating the influence of older events (e.g., effectively approximating a high-pass filter in time)))), dust-sensitive squared differences (e.g., for PM sensors (e.g., (PM₁₀−PM_{2.5})², (PM_{2.5}−PM₁)²) (e.g., emphasizes large size-bin disparities during dust events (e.g., these terms may improve performance by correcting PM_{2.5} underestimation)))), ratio features (e.g., PM_{2.5}/PM_{1.0}, PM_{1.0}/PM₁₀) (e.g., captures relative shifts in particle-size distribution (e.g., distinguishing coarse dust from fine combustion), interaction terms (e.g., PMraw_{2.5}×RH, v_{Gas}×T) (e.g., handles situations where two variables jointly distort the sensor signal more than each alone (e.g., high humidity+high temperature causing extra baseline drift)), rolling window statistics (e.g., 14-day average of NO₂, 20th/80th percentiles of recent measurements, days since deployment (e.g., helps the model learn and compensate for gradual drift over time and normalize out slow seasonal trends)), and/or the like) may be chosen in any suitable manner, including, but not limited to, physical insight (e.g., knowing that humidity affects gas sensor baselines with a characteristic time constant suggests using an exponential filter for RH), empirical testing (e.g., collocation case studies (e.g., during dust events) may reveal which transformations, such as squared PM differences, significantly improve R\hat{ }2), cross-validation (e.g., features may be added or removed based on whether they improve performance on held-out data without overfitting), and/or the like. Models can be physics-based, machine learning-based, or hybrids, and/or may be trained to minimize discrepancies from reference values. Cross-validation techniques may be employed to prevent overfitting and/or to ensure reliability in diverse conditions.

Global calibration may be configured to enable out-of-the-box accuracy for sensors, which may be particularly useful in regions where reference monitors are unavailable. These calibrations may be developed from massive datasets of collocated sensors across diverse locations (e.g., diverse cities) and climates, using any suitable models like LightGBM and/or stepwise multivariate linear regression. Advanced features, such as humidity-adjusted baselines and/or dust-event corrections using squared differences and/or particle ratio transformations, may be utilized to enhance accuracy. Some embodiments may combine global models through hybrid approaches, such as by blending machine learning and linear regression outputs using unsupervised weighting methods to optimize calibration under varying conditions.

Collocation-based calibration may involve placing sensors next to reference monitors for extended periods, such as a month or any other suitable period of time, to ensure exposure to representative environmental conditions. Data collected during such period(s) may be used to train models that may be configured to adjust sensor outputs to match reference standards. These calibrations may be implemented in real-time or applied retroactively and may be validated with statistical metrics like the Pearson correlation coefficient (“R²”) and/or root mean square error (“RMSE”). Periodic recalibration may be recommended to maintain accuracy as environmental conditions evolve.

This disclosure also describes a layered calibration strategy that may integrate global and local collocation-based calibrations. Sensors may first be normalized to reduce manufacturing variability (e.g., during sensor production). For example, multiple sensor nodes (e.g., at the factory outdoors or in a controlled chamber, or at an initial side-by-side outdoor setup (e.g., in a parking lot near the envisioned deployment region), and/or the like) may be normalized so that each sensor's raw output may be aligned to the group mean or a representative sensor. That may reduce device-to-device variation before any global or local calibration is applied. This operation can also be skipped. Then, sensors may be globally calibrated to ensure a consistent baseline (e.g., prior to sensor distribution for end-use). There may be many ways to apply a global calibration to the raw output of a sensor. After production and optional normalization, or at any point after deployment, each sensor's firmware may be loaded with a global calibration so that it can use it to calibrate raw measurements. Alternatively, a cloud pipeline may be configured to apply a global calibration to the output of a sensor after receiving its raw data and publish the result as calibrated data to a dashboard, API, or data storage in real-time. Alternatively, the global calibration can be applied asynchronously to the collected sensor raw measurements, in post processing. Finally, project-specific collocation may be used to fine-tune the outputs (e.g., when the sensor is positioned in its end-use environment. Once the optionally normalized and globally calibrated sensor arrives at its final site, it can undergo an optional collocation with a local reference. Note that this may involve installing the sensor next to a reference monitor for some time before it is moved to its final monitoring location. The resulting collocation-based calibration (e.g., often a simpler regression or scaling factor) may then be layered on top of the global calibration, meaning that the output of the global calibration may be used as an input to the additional collocation-based calibration. That operation may tailor the output to local sources, environmental conditions, and/or pollutant composition. This layered approach may enhance accuracy, support efficient calibration transfer across a sensor network, and/or enable dynamic updates based on ongoing collocation data.

In the specific case of black carbon monitors, a method of the disclosure may introduce a calibration based on the rate of temperature change. During production, monitors may be exposed to controlled temperature cycles under clean air conditions, and regression coefficients may be derived. These coefficients may be later used to correct sensor outputs during field operation. In some embodiments, constants may be parameters that may be used to compute derived features (e.g., how fast the RH baseline decays). They may remain fixed once set and may be part of the feature-engineering stage (e.g., deltaRhConst and τ for RH baseline filtering, c₁-c₃ for a temperature baseline polynomial in gas sensor, etc.). In some embodiments, model weights or regression coefficients may be learned during model training to map features to a target pollutant concentration (e.g., in a linear regression, these may be the slopes and intercepts, in an ML ensemble (e.g., LightGBM), the weights may refer to leaf values in each decision tree or to regularization parameters, etc.). In some embodiments, other coefficients may be utilized, such as sensitivity coefficients (ecsSensitivity), which may be provided by the manufacturer and used to convert gas sensor voltage to a preliminary ppb estimate, Pearson correlation coefficient (R²), which may be a performance metric, not part of model internals, and/or the like. Therefore, feature-engineering constants (e.g., deltaRhConst, tau, polynomial coefficients, etc.) may not be the same as the model's learned weights. They may be pre-determined or hand-tuned constants. Model weights/regression coefficients may be learned by fitting data. They may be called “calibration coefficients” when describing a final calibration equation. Therefore, although both may use the term “coefficient,” they may occupy different roles: one set may create the inputs (e.g., features), and the other set may map those inputs to an output. This may ensure that measurements may remain stable and accurate despite rapid environmental shifts. The technique described may be something that solves an issue with black carbon monitors when deployed outdoors in a small enclosure without temperature control. A key aspect may be the fact that the baseline and temperature rate of change can be measured while applying a particle filter at the inlet (e.g., zero black carbon concentration), instead of characterizing the instrument response to temperature rate of change at different black carbon concentrations, which may make it easy to operationalize during production. When creating an easy-to-deploy-outdoor black carbon monitor, this is a problem that may need to be addressed.

Additionally, this disclosure presents a modular system where a sensor node, which may be capable of measuring pollutants, can be expanded with one or more add-on or accessory modules for additional capabilities like BC monitoring. The system may be configured to support any suitable third-party integration, such as through standardized communication protocols, and/or may be configured to use any suitable cloud software for remote configuration and calibration. Powering options may include, but are not limited to, solar energy, thereby making the system suitable for remote deployments. A method of the disclosure may also be proposed for enhancing PM mass concentration estimates using BC source attribution, thereby further improving measurement reliability.

More broadly, beyond any specific features and model architectures described herein, a core of this disclosure may lie in a generalizable process of developing global calibration. This process may include (1) collecting large-scale collocation datasets from air quality sensors deployed alongside reference instruments across diverse environmental conditions, (2) computing a set of derived features that capture environmental influences, sensor behaviors, and interactions among variables (e.g., the derived features may capture reasons for poor alignment with reference-grade monitors, such as, for example, environmental interferences, sensor-to-sensor variability, changes in pollutant composition, changes in environmental conditions, sensor drift, sensor-to-sensor variability, and/or the like), and (3) training a machine learning model (e.g., LightGBM or any other suitable regression or ensemble technique) to produce calibrated outputs that are significantly more accurate than the raw sensor measurements. This methodology can be adapted to different sensor types, pollutants, and/or deployment use cases (e.g., the process may be repeated for each new type of sensor and pollutant, but not necessarily for each deployment use case), and may represent a flexible, scalable solution to improving the data quality of low-cost air quality monitoring networks.

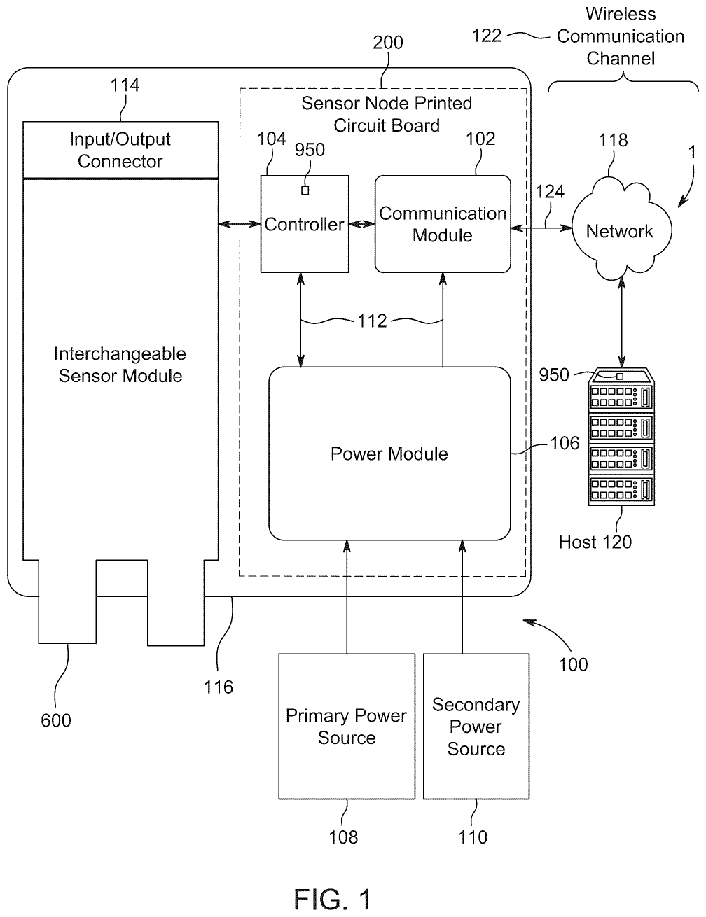

### FIGS. 1-13

FIG. 1 is a high-level block diagram of a system 1 that may include a sensor node 100 according to certain embodiments of the disclosed subject matter. The sensor node 100 may include a communication module 102, a controller 104, a power module 106, a primary power source 108, a secondary power source 110, a reliable power supply 112, an input/output connector 114, an enclosure 116, a printed circuit board 200, and an interchangeable sensor module 600.

The communication module 102 may be configured to establish a wireless communication channel 122 over a network 118 of system 1 with a host 120 of system 1. “Host” may refer to any computing device or computer device or computer system configured to send and receive commands. Examples of a host include, but are not limited to, a computer, a laptop, a mobile device, an appliance, a virtual machine, an enterprise server, a desktop, a tablet, a main frame, and the like. “Wireless communication channel” may refer to a communication media configured to exchange information in the form of structured data between a sender and a receiver. A wireless communication channel includes a communication channel for which one or more of the links in the channel is between two components that are not connected by an electrical conductor. One example of a wireless communication technology is radio waves, but other forms of electromagnetic waves may be used. (“Wireless.” Wikipedia. Sep. 9, 2019. Accessed Sep. 9, 2019. https://en.wikipedia.org/wiki/Wireless.)

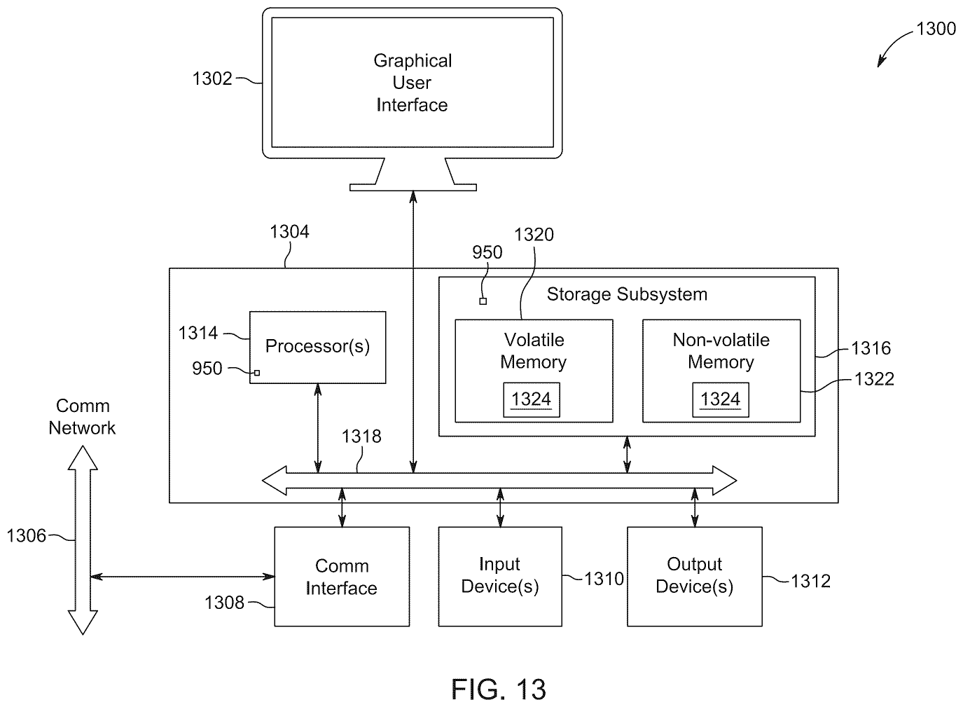

The network 118 may be a communication network 1306 and the host 120 may be a computing device 1300 as illustrated in FIG. 13. The communication module 102 may further be configured to receive instructions from a data management platform to operate a sensor module. In one embodiment, the data management platform is operating on a host 120. The communication module 102 may receive a command to operate the sensor module per the instructions and may send a reading to the data management platform.

The controller 104 may be configured to manage the interchangeable sensor module 600 and send measurement data 124 to the host 120 by means of the wireless communication channel 122. “Controller” may refer to any hardware, device, component, element, circuitry, or circuit configured to manage and control another software, hardware, firmware, or logic unit, component, device, or component. The controller 104 may store instructions (e.g., with any suitable memory) to operate the interchangeable sensor module 600. The controller may receive a first current from a power source and may then operate the interchangeable sensor module 600 in response to a command.

The interchangeable sensor module 600 may be atmospherically isolated from the communication module 102 and the controller 104. This may be accomplished though O-rings or other seals surrounding openings in the body of the interchangeable sensor module 600. Holes necessary to mount or otherwise affix the interchangeable sensor module 600 within the sensor node 100 may be similarly sealed or located on tabs on the periphery of the interchangeable sensor module 600, such that the holes do not cause an incursion into the body of the interchangeable sensor module 600.

The interchangeable sensor module 600 may include one or more air quality sensors. The interchangeable sensor module 600 may receive a second current from the power source and may operate a fan utilizing the second current in response to the command to direct an aerosol stream, such as a gas, from an ambient environment external to an inlet port, to the one or more air quality sensors, and out of an outlet port. The interchangeable sensor module 600 may operate each of the one or more air quality sensors to generate a series of measurements before, during, or after operation of the fan and may generate the reading for each of the one or more air quality sensors from the series of measurements. The reading may then operate the data management platform to generate a measurement by selecting a co-location pair for the sensor node based on a location of the sensor node, determining a calibration model from the co-location pair, and generating the corrected measurement by applying the calibration model to the reading, the data management platform storing the reading and the corrected measurement. “Co-location pair” may refer to a pair of sensors including at least one sensor node and one reference monitor positioned within a distance limit from each other. The distance limit is defined such that, if the distance between a reference monitor and a sensor node is at or less than the distance limit, the reference monitor and the sensor node are considered to be exposed to the same concentration of gas(es) and/or gas pollutants such as air pollutants.

The power module 106 may be configured to supply a reliable power supply 112 from a primary power source 108, such as a solar panel, supplemented by a secondary power source 110, such as a battery. The battery may be rechargeable, such that while enough power is available from the primary power source 108, the secondary power source 110 or battery may be recharged, storing the excess solar energy for later use. “Power module” may refer to any hardware, device, component, chip, element, circuitry, or circuit configured to manage how much electrical power is provided to a circuit, circuitry, system, or subsystem. In one embodiment, a power module is a circuit of electrical components organized and housed within a single chip or other electrical component. In one embodiment, the power module is configured to constantly monitor current and/or voltage use and automatically connect a battery when the current and/or voltage used by a connected circuit drops below a threshold level. In one embodiment, the power module is configured to automatically charge a connected battery when the current and/or voltage supplied by a primary power source exceeds the current and/or voltage drawn by a connected circuit. In one embodiment, the power module may include a battery charger with power path management such as those available from Microchip Technology Inc. Of Chandler Arizona and may include other components such as a buck-boost converter, a battery monitor, and the like. “Power source” may refer to a source of electrical energy for one or more electrical circuits connected to the power source. “Reliable power supply” may refer to electrical energy converted from electrical potential energy at a specific rate per unit of time that is maintained at the specific rate per unit of time within an acceptable tolerance level for proper operation of one or more electrical circuits connected to the reliable power supply and which electrical circuits provide an electrical load. “Primary power source” may refer to a power source for one or more electrical circuits that an electrical design of the one or more electrical circuits expects to be available a majority of the time and is designed to provide a majority of the electrical energy used by the one or more electrical circuits. “Secondary power source” may refer to a power source for one or more electrical circuits that an electrical design of the one or more electrical circuits expects to be available less than a majority of the time and is expected to provide less than a majority of the electrical energy used by the one or more electrical circuits.

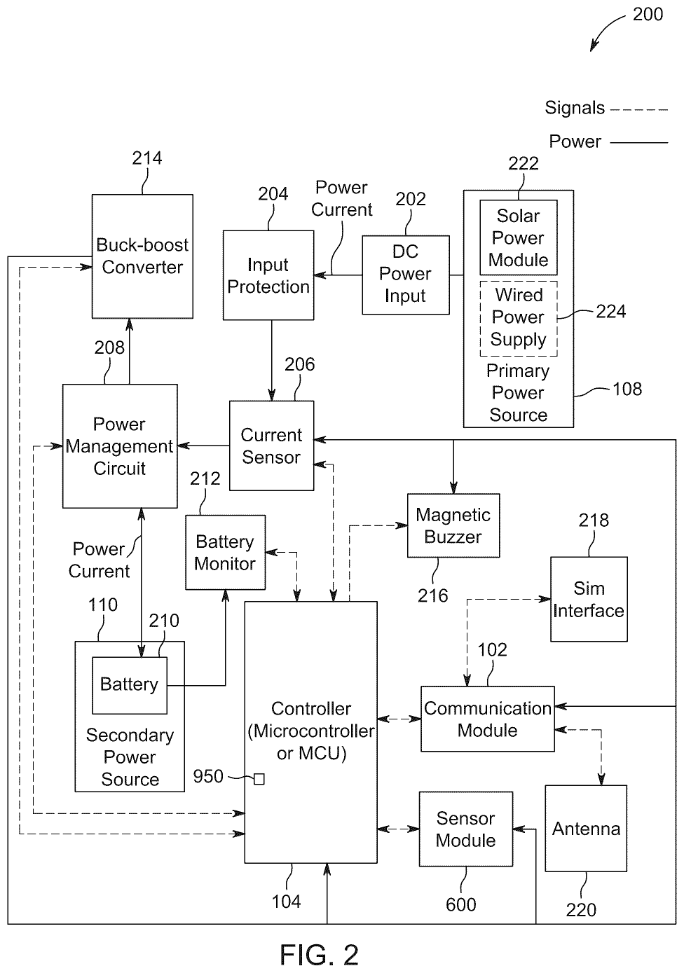

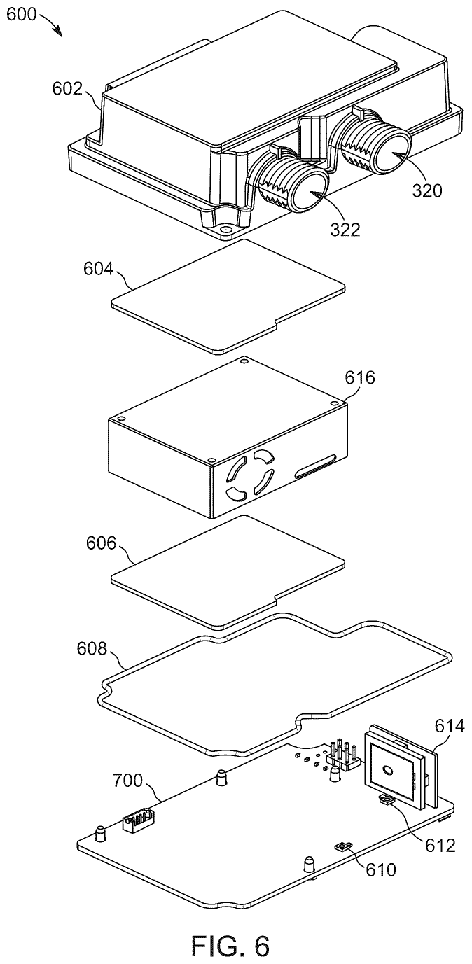

A printed circuit board 200 may be configured to interconnect the communication module 102, controller 104, and power module 106. The printed circuit board 200 may also include an input/output connector 114 configured to permit an interchangeable sensor module 600 to be coupled to the printed circuit board 200 for ease of maintenance, repair, or upgrade. Additional information regarding the printed circuit board 200 is provided with regard to FIG. 2. The interchangeable sensor module 600 is described in more detail with regard to FIG. 6.

An enclosure 116 may be configured to have walls forming an enclosed space, the walls having an inlet and an outlet aligned with inlet and outlet ports of the interchangeable sensor module 600. The enclosure 116 may house the printed circuit board 200, the interchangeable sensor module 600, and all other sensors and associated components, providing protection from environmental conditions as well as providing an isolated internal environment to facilitate accurate sensor readings. The modularity of the sensor node may enable the sensor node to be configured differently depending on deployment scenarios to ensure scalable deployment of a dense sensor network in a region where air quality is measured.

FIG. 2 illustrates a printed circuit board 200 in accordance with one embodiment. The printed circuit board 200 may include a DC power input 202, an input protection 204, a current sensor 206, a power management circuit 208, a battery 210, a battery monitor 212, a buck-boost converter 214, a controller 104, a magnetic buzzer 216, a communication module 102, a SIM interface 218, an antenna 220, and a sensor module printed circuit board 700.

DC power input 202, input protection 204, current sensor 206, power management circuit 208, battery 210, battery monitor 212, buck-boost converter 214, and other components such as low-dropout regulators may be used to realize the power module 106 illustrated in FIG. 1.

The power management circuit 208 may be configured to monitor an electrical load and maintain the reliable power supply by selectively supplying supplemental power from the secondary power source, such as the battery 210, in response to the primary power source, such as a wired power supply 224 or solar power module 222, supplying power below a threshold. “Wired power supply” may refer to a power source that provides power by way of an electrical conductor. In one example embodiment, a wired power supply is an alternating current available over a power grid for a community or city delivered over a power network, which is converted to a direct current power supply by a wired power supply component such as an AC power adapter.

The power management circuit 208 may also be configured to charge the battery 210 from the DC power input 202 when enough power is available. In some embodiments, primary power may be supplied by the battery. Solar power may function as a secondary power source to recharge the battery.

Controller 104 may be a microcontroller (MCU) that may control the power module, receive and process a plurality of statuses and measurements from the power module, and communicate with the interchangeable sensor module 600, the communication module 102 and other external hosts through various serial communication protocols. Means of connection to external hosts may include a programming header, a debugging header, reset control, a SIM interface 218, and an antenna 220.

The device may provide audible feedback when it is set up and/or connected to the network. This audible feedback may be provided by means of a magnetic buzzer 216 powered by the power module and controlled by the controller 104. The device may provide audible notifications indicating that it has successfully powered on, that it has successfully connected to a network, that it has failed to connect with the network, and when its battery is low. Installation and configuration may in some embodiments be facilitated by interaction with an application available online, on a host device, or via a mobile application on a mobile device.

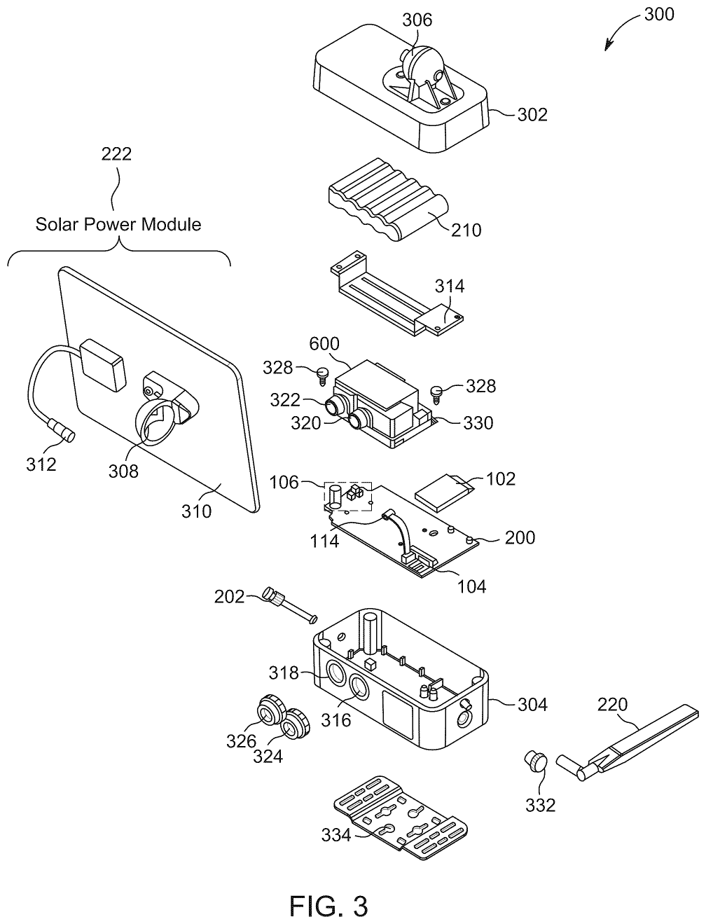

FIG. 3 illustrates an exploded view of a sensor node 300 in accordance with one embodiment. The sensor node 300 includes an enclosure 116 that may be configured as an enclosure lid 302 and an enclosure body 304. The enclosure lid 302 may include a gimbal fastener 306 which engages a solar panel mounting structure 308 on a solar power module 222 in order to integrate the solar power module 222 with the system. The gimbal fastener 306 may allow for the orientation of the solar panel mounting structure 308 to be adjusted such that maximum exposure of the solar panel 310 to direct sunlight is achieved. Such an adjustment may be made by the installer or by a user at the location where the sensor node 300 is installed.

The solar power module 222 may provide primary power to the printed circuit board 200, as discussed in detail with regard to FIG. 2, by connecting to the power module 106 of the printed circuit board 200 by means of a solar panel electrical coupling 312 connected to the DC power input 202. Additionally, secondary power may be provided by a battery 210, secured in the enclosure 116 with a battery mount 314. The enclosure may also in some embodiments include a separate battery compartment. Some embodiments may include an AC power connection in addition to or instead of either or both of the solar power module 222 and battery 210.

The printed circuit board 200 may be configured as an interface between the communication module 102, controller 104, power module 106, and interchangeable sensor module 600 as described with regard to FIG. 1 and FIG. 2. The communication module 102 may send data to an external host by means of the antenna 220.

The enclosure body 304 may be configured with a first opening 316 and a second opening 318, sized and positioned to align with the inlet port 320 and outlet port 322 of the interchangeable sensor module 600, respectively. The openings may allow airflow to reach the interchangeable sensor module 600 through an inflow filter 324 and an outflow filter 326. The first opening 316 and inflow filter 324 may receive air samples from the environment. After the air sample has been processed through, for example, the interchangeable sensor module 600 the air sample may be returned to the environment through the second opening 318 and the outflow filter 326. The interchangeable sensor module 600 may attach to the printed circuit board 200 by means of removable fasteners 328 placed through holes 330 for removable fasteners incorporated into the body of the interchangeable sensor module 600.

The enclosure body 304 may also be configured with a vent 332. A node mount 334 may attach to the enclosure body 304, allowing the sensor node 300 to be mounted to a structure using the node mount 334. The node mount 334 may include a plurality of mounting holes for screws, nuts, bolts, or other fastening devices.

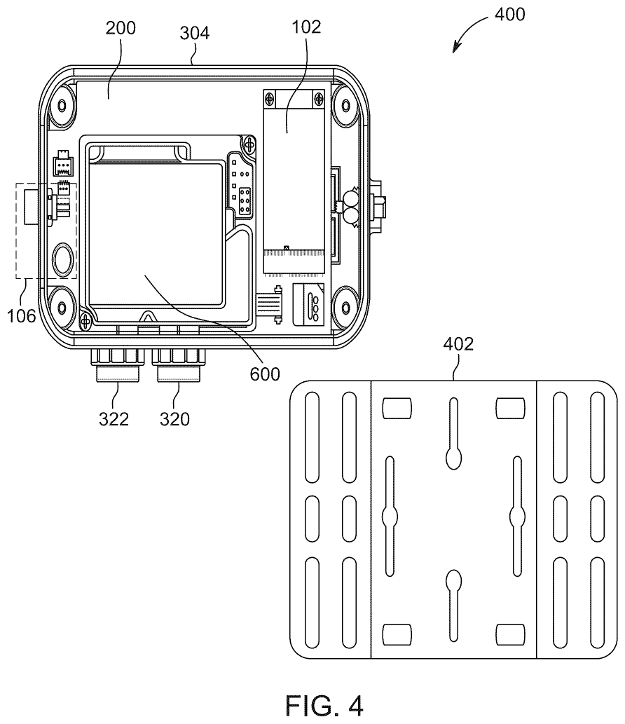

FIG. 4 illustrates a top view of a sensor node 400 with the enclosure lid 302 removed, in accordance with one embodiment. The sensor node 400 includes an enclosure body 304 that encompasses an interchangeable sensor module 600 and a printed circuit board 200 that includes a communication module 102, a power module 106 and a controller. The sensor node 400 may additionally include a universal mount 402 configured to mount the enclosure body 304 in a plurality of mounting configurations. “Universal mount” may refer to a plate, bracket or the like configured with one or more holes and/or one or more slots arranged and sized to accept one or more fasteners to secure the mount to a wall, fence, or pole and to secure a sensor node to the universal mount.

The printed circuit board 200 with communication module 102, controller, and power module 106, may be substantially the same as those illustrated in FIG. 1 and FIG. 2. The power module 106 may be configured to accept power from a solar power module that may supply current as a primary power source. The power module 106 may also be configured to accept power from a battery configured to selectively supply current as a primary power source and/or to supplement power supplied by the solar power module.

The interchangeable sensor module 600 may be configured to monitor an air sample for one or more characteristics, such as air quality or concentrations of specific gases or particulates. “Characteristic” may refer to any property, trait, quality, or attribute of an object or thing. (“characteristic” Merriam-Webster.com. Merriam-Webster, 2019. Web. 27 Aug. 2019.) Examples of characteristics include, but are not limited to, chemical composition, water content, temperature, relative humidity, particulate count, contaminant count, and the like.

The interchangeable sensor module 600 may include an inlet port 320 and an outlet port 322. In some embodiments, the interchangeable sensor module 600 may be an air quality sensor. Air quality sensors may include an airflow structure, a particle counter, and at least one other air quality sensor. Such a sensor module may form an airflow structure to direct an aerosol stream to the at least one other air quality sensor before directing the aerosol stream to the particle counter. See FIG. 6 for additional detail.

The enclosure body 304 may be configured with openings sized and positioned to align with the inlet port 320 and outlet port 322 of the interchangeable sensor module 600, respectively. The openings may allow airflow to reach the interchangeable sensor module 600 through an inflow filter and an outflow filter. The inlet port 320 may receive air samples from the environment through the opening in the enclosure body 304 and the inflow filter. After the air sample has been processed through, for example, the interchangeable sensor module 600, the air sample may be returned to the environment through the outlet port 322 through the opening in the enclosure body 304 and the outflow filter.

In addition to the enclosure body 304 and the enclosure lid 302 (shown in FIG. 3), the enclosure 116 including the enclosure lid 302 and enclosure body 304 may also include one or more seals. The enclosure lid 302 may fasten to the enclosure body 304 by means of removable fasteners. “Removable fastener” may refer to any fastener configured to be removed using conventional tools such as a screwdriver, a wrench, a nut driver, or the like. Removable fasteners may be made of a variety of materials including metal, plastic, composite materials, metal alloys, plastic composites, and the like. Examples of removable fasteners include, but are not limited to screws, bolts, nuts, posts, pins, thumb screws, and the like.

The seals, openings, and enclosure lid 302 may be configured to engage the enclosure body 304 to provide a liquid ingress protection rating greater than four. “Ingress protection rating” may refer to a rating system that defines the level that an enclosure protects internal components from ingress of solid objects, liquids, and gases. Engineering ToolBox, (2003). IP-ingress protection rating. [online] Available at: https://www.engineeringtoolbox.com/ip-ingress-protection-d_452.html 5 Sep. 2019. In this manner, the electronics within the sensor node 400 may be isolated and protected from environmental conditions. In another embodiment, the electronics of the sensor node 400 may be isolated from the air sample chambers such that only the air sample chambers are exposed to a gas mixture sample.

In another embodiment, the enclosure may include a body and a lid, wherein the lid is permanently connected to the body. In this embodiment, the enclosure may include an opening and a door configured to seal the opening from moisture ingress when the door is closed. This opening may be sized to slidably accept the interchangeable sensor module 600.

In some embodiments, the sensor node 400 may further include a second interchangeable sensor module configured to monitor one or more environmental characteristics. The second interchangeable sensor module may be configured to sit within the enclosure and may removably couple to the controller and the power module.

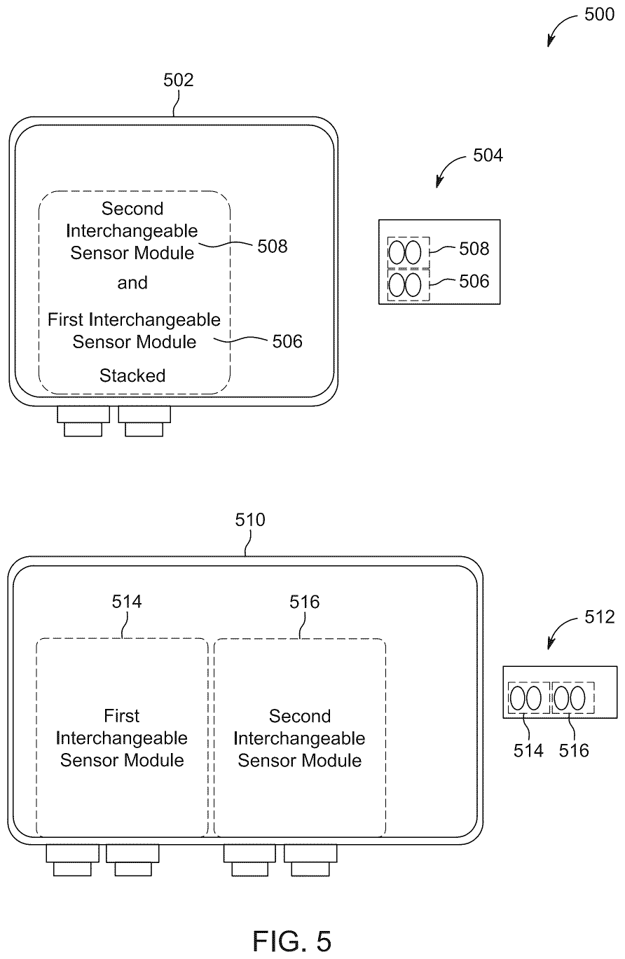

FIG. 5 illustrates dual sensor module configuration 500 in accordance with some embodiments. The sensor node may include an enclosure configured to accommodate two interchangeable sensor modules.

A top view of an enclosure for two stacked sensor modules 502 is shown with dotted lines indicating the footprint that a stacked first interchangeable sensor module 506 and second interchangeable sensor module 508 would occupy within the enclosure. A side view of an enclosure for two stacked sensor modules 504 is also illustrated, showing the second interchangeable sensor module 508 stacked atop the first interchangeable sensor module 506. This is one embodiment, and is not intended to limit the possible configurations.

A top view of an enclosure for two side-by-side sensor modules 510 is also shown, along with a side view of an enclosure for two side-by-side sensor modules 512. Both enclosures 510/512 are configured to house a first interchangeable sensor module 514 and a second interchangeable sensor module 516 arranged side by side. This is one embodiment, and is not intended to limit the possible configurations.

FIG. 6 illustrates an interchangeable sensor module 600 in accordance with one embodiment. “Interchangeable sensor module” may refer to a sensor module specifically designed to be removed from a larger unit, system, subsystem, or the like. In certain embodiments, the interchangeable sensor module includes one or more holes or openings each configured to receive a removable fastener such as a screw, a bolt, a pin, or the like. In certain embodiments, the interchangeable sensor module includes one or more clips, tabs, slots, or other structural components configured to engage corresponding structural components of a housing or bracket of a larger system or subsystem, such that the interchangeable sensor module is configured to engage and disengage with the housing or bracket to install or remove the interchangeable sensor module. The interchangeable sensor module 600 includes an airflow structure 602, an inlet port 320, an outlet port 322, a top buffer 604, a bottom buffer 606, an O-ring 608, a sensor module printed circuit board 700, a sensor 610, a sensor 612, a sensor 614, and a particle counter 616.

The airflow structure 602 may be airtight with the help of the O-ring 608 such that any air samples are fully contained and analyzed within the airflow structure 602. The interchangeable sensor module 600 may include at least one air quality sensor with an active sampling mechanism, such as a fan or a blower. The interchangeable sensor module 600 may include an inlet port 320 for drawing in the air sample and an outlet port 322 for expelling the sampled air.

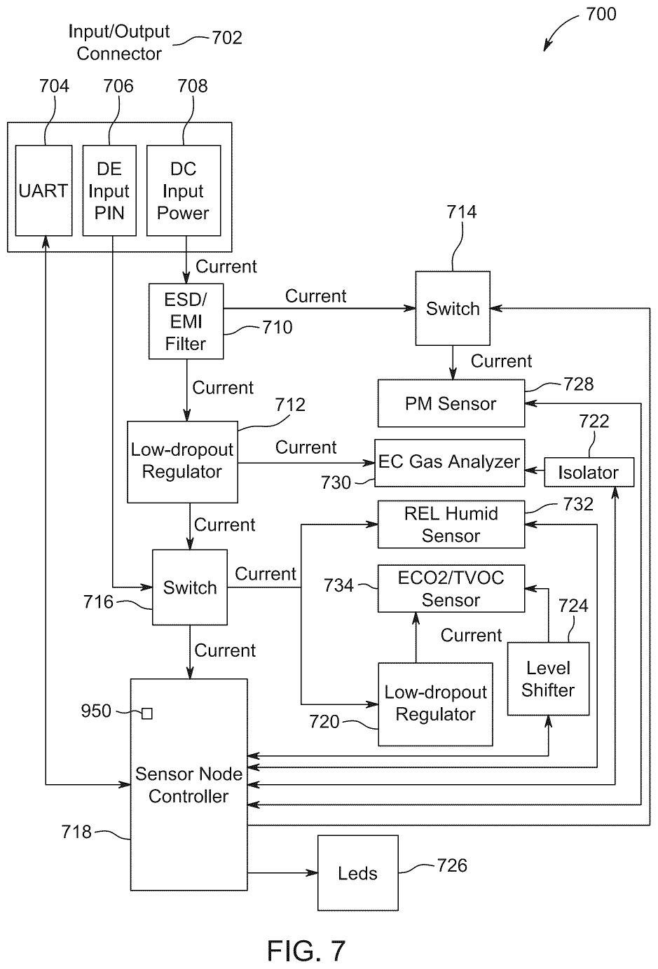

The sensor module printed circuit board 700 may include and/or connect a plurality of electrical components configured to perform the sensing functions necessary to detect and analyze air quality conditions. The sensor module printed circuit board 700 is illustrated in more detail in FIG. 7.

The interchangeable sensor module 600 may include a plurality of air quality sensors such as a sensor 610, a sensor 612, and a sensor 614, which may measure the concentration of air pollutants. These sensors may be located in the airflow structure 602 and mounted to the sensor module printed circuit board 700.

The structure of the interchangeable sensor module 600 and the placement of the air quality sensors within the interchangeable sensor module 600 may be configured in such a way that the active sampling mechanism of one of the air quality sensors is used to expose all air quality sensors in the interchangeable sensor module 600 to samples of air from the ambient environment.

The particle counter 616 may be sandwiched between the top buffer 604 and the bottom buffer 606 to reduce vibration and electrically isolate its metal chassis from the sensor module printed circuit board 700. The enclosure illustrated in FIG. 3 and FIG. 4 may be configured to atmospherically isolate the interchangeable sensor module 600 from the power module, communication module, and controller. The sensor node may include removable fasteners configured to engage the enclosure and the interchangeable sensor module 600.

A modular air sensor or gas sensor design may be employed, such that a range of sensor modules may be connected to each sensor node. Sensor modules may be easily installed, removed, and replaced for repair or upgrade, or may provide a range of sensor types to measure specific air components in specific locations. Such swappable sensor modules may connect to the sensor node PCB by means of a standard connector.

FIG. 7 illustrates a sensor module printed circuit board 700 in accordance with one embodiment. The sensor module printed circuit board 700 may include an input/output connector 702, a universal asynchronous receiver/transmitter 704, a digital enable input pin 706, a DC input power 708, an electrostatic discharge and electromagnetic interference filter 710, a low-dropout regulator 712, a switch 714, a switch 716, a sensor node controller 718, a low-dropout regulator 720, an isolator 722, a level shifter 724, light emitting diodes 726, a particulate matter sensor 728, an electrochemical gas analyzer 730, a relative humidity sensor 732, and an equivalent carbon dioxide and total volatile organic compounds sensor 734. Therefore, any suitable sensor node or sensor device (e.g., node 100, 902, etc.) may include any suitable number of sensor components (e.g., sensor component 610, 612, 614, 728, 732, etc.).

In another embodiment, a sensor module may contain multiple air quality sensors. The aforementioned components can be used to realize a sensor module that may gather sensor measurements from a plurality of sensors and send the sensor measurements to a host device (such as a sensor node) upon request via serial interface.

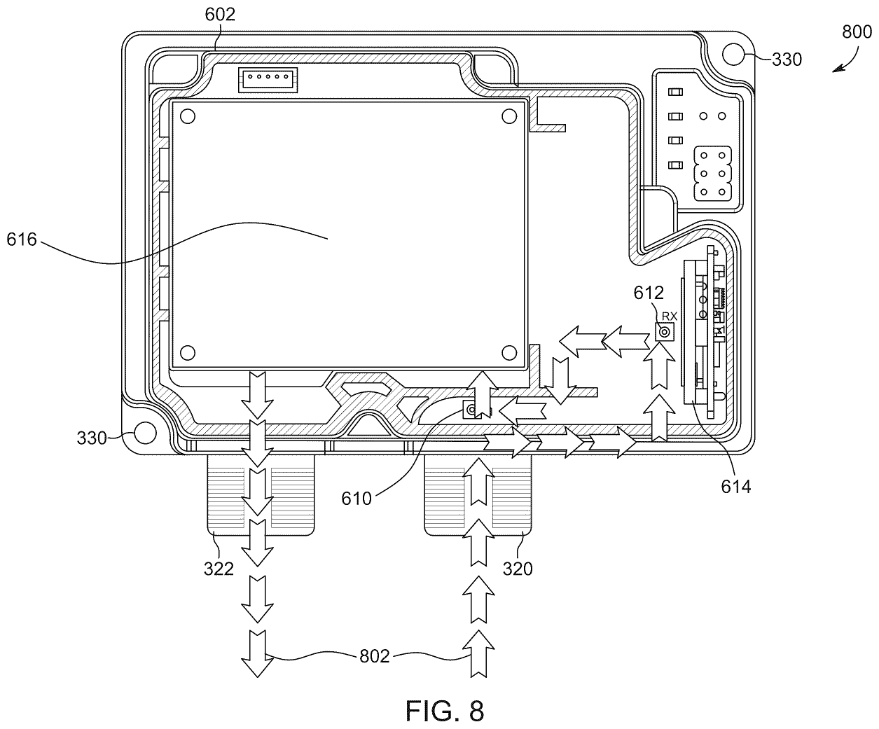

FIG. 8 illustrates a cross-sectional view of a sensor module 800 in accordance with one embodiment. The sensor module 800 includes an airflow structure 602, an inlet port 320, an outlet port 322, a sensor 610, a sensor 612, a sensor 614, and holes 330 for removable fasteners.

The enclosure encompassing the sensor module 800 may be configured to atmospherically isolate the sensor module 800 from the power module, communication module, and controller. The holes 330 may be configured to accept removable fasteners such as screws to engage the enclosure and the holes 330. These holes 330 may be located on tabs which extend at the periphery of the interchangeable sensor module 600 body, and in this manner provide mounting holes that do not cause an incursion into the interchangeable sensor module 600 body, and thus do not need to be sealed to maintain atmospheric isolation of the sample being tested. Other forms of connectors or fasteners other than holes 330 and/or removable fasteners may be used for coupling the sensor module 800 to the enclosure.

To change out an interchangeable sensor module, a user may open the enclosure of the sensor node to access the sensor module 800. This may be accomplished by removing the enclosure lid in some embodiments. In other embodiments not illustrated, the enclosure may be configured with a sensor module aperture with a latched and sealed door, or some other configuration allowing access to the sensor module 800 while providing adequate isolation from the environment around the sensor node.

After accessing the sensor module 800, The user may remove the removable fasteners (e.g., screws, bolts, latches, or other fastening devices), thus releasing the sensor module 800 from mounting hardware incorporated into the enclosure or sensor node PCB. The user may attach a new interchangeable sensor module in place of the one removed, securing it by replacing the removable fasteners through the holes 330 located on the new sensor module. This is only one embodiment. Interchangeable sensor modules may alternatively incorporate captive fasteners that may be disengaged from the enclosure and/or sensor node PCB but remain attached to the sensor module. The sensor node may alternately include a latching mechanism holding interchangeable sensor modules in place when installed. Any combination of these methods may be used to secure the sensor module 800 in place within the sensor node while allowing easy changeout to facilitate repairs or upgrades.

An airflow path 802 is illustrated, using black arrows to show how air from the surrounding environment passes through the sensor module 800. The airflow may travel in through the inlet port 320 into a series of baffles designed into the airflow structure 602, configured to direct the air over at least one air quality sensor. In the illustrated embodiment, the air is directed across sensor 614, then passes over sensor 612. The air is then directed in the opposite direction to pass over sensor 610 before entering the particle counter 616. After passing through the particle counter 616, the air exits the airflow structure 602 and flows out through the outlet port 322.

In this manner, and due to the isolation provided by the airflow structure 602, as well as the O-ring 608, and the enclosure itself, the volume of air being sampled, tested, and analyzed, may remain isolated from the environment both inside and outside the enclosure.

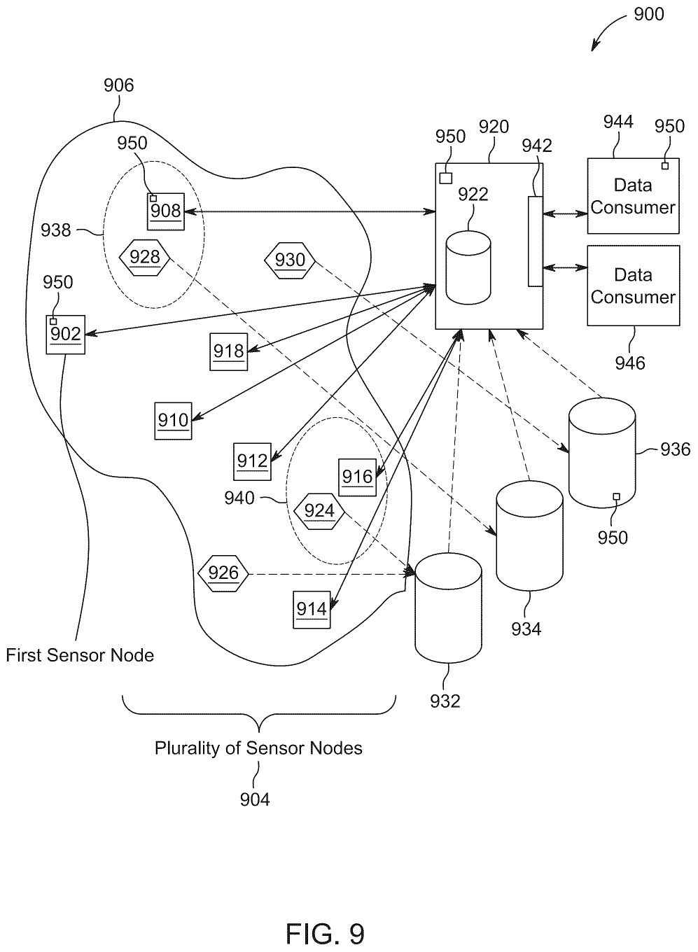

FIG. 9 is a system diagram illustrating a hyperlocal air quality monitoring system 900 deployed in a region 906 in accordance with certain embodiments of the disclosed subject matter.

The hyperlocal air quality monitoring system 900 may include a first sensor node 902, a plurality of sensor nodes 904, and a region 906. The plurality of sensor nodes 904 may include a sensor node 908, a sensor node 910, a sensor node 912, a sensor node 914, a sensor node 916, and a sensor node 918. The hyperlocal air quality monitoring system 900 may further include data management platform 920, a storage control memory structure 922, a reference monitor 924, a reference monitor 926, a reference monitor 928, a reference monitor 930, a data control memory structure 932, a data control memory structure 934, a data control memory structure 936, a co-location pair 938, a co-location pair 940, a data interface 942, a data consumer 944, and a data consumer 946. Any number of sensor nodes, monitors, data control memory structures, etc., may be utilized herein, and the number is not limited to the ones in FIG. 9.

The hyperlocal air quality monitoring system 900 may be configured to implement the method disclosed herein. A first sensor node 902 may be placed near a reference monitor 924 within a region 906. A plurality of sensor nodes 904 may then be placed at various locations within the region 906. Measurement data may be gathered from the first sensor node 902, the reference monitor 924, and the plurality of sensor nodes 904. A calibration profile may be determined for each of the first sensor node 902 and the plurality of sensor nodes 904 based on measurement data from the reference monitor 924. In one embodiment, the calibration profile may be determined for each of the first sensor node 902 and the plurality of sensor nodes 904 based on measurement data from the reference monitor 924 and measurement data from the first sensor node 902. The calibration profile for each of the first sensor node 902 and the plurality of sensor nodes 904 may be applied to measurement data from each of the first sensor node 902 and the plurality of sensor nodes 904 to obtain calibrated measurement data for each of the sensor nodes.

In one embodiment, the hyperlocal air quality monitoring system 900 deployed in region 906 may include multiple sensor nodes (the first sensor node 902, the sensor node 908, the sensor node 910, the sensor node 912, the sensor node 914, the sensor node 916, and the sensor node 918) deployed at known locations within region 906 where it is desired to measure air quality, and a data management platform 920. The sensor nodes periodically acquire air quality measurements and communicate said measurements to the data management platform. The data management platform is configured to receive information from the sensor nodes and store it in a storage control memory structure 922. As a non-limiting example, the sensor nodes are low-cost air quality sensors that communicate air quality measurements to the data management platform 920 wirelessly through a data network, and the data management platform 920 is a combination of controllers, data processors, software services, control memory structures, and the like. As a non-limiting example, it is desired to measure the air quality at several outdoor locations in a city, and the sensor nodes are mounted at those locations to city furniture, building walls, or other infrastructure.

Reference monitors (a reference monitor 924, a reference monitor 926, a reference monitor 928, and a reference monitor 930) are found at known locations within region 906 and periodically acquire air quality measurements and publish said measurements to data control memory structures (a data control memory structure 932, a data control memory structure 934, and a data control memory structure 936). “Reference monitor” may refer to a gas monitor such as an air monitor approved by a municipal, state, or other governmental agency for use in tracking the composition and make up of air in a given location. A reference monitor is calibrated and certified to provide measurement data that is accurate and reliable for determining a quality measure for air in the region serviced by the reference monitor.

The data management platform 920 is configured to periodically retrieve information from the data control memory structures and store it in the storage control memory structure 922. As a non-limiting example, the monitors are highly accurate air quality monitoring stations operated by governmental agencies or other organizations, with measurement accuracy that may be higher than the accuracy of the sensor nodes, and the data control memory structures are online data sharing platforms where governmental agencies or other organizations openly publish air quality monitoring information for divulgation with the public. The data management platform 920 is configured to perform processes aimed at increasing the accuracy of the measurements acquired by the sensor nodes.

Some sensor nodes may be purposefully deployed at close proximity to some monitors with the aim of increasing the accuracy of the measurements acquired by the sensor nodes. Pairs of sensor nodes and monitors with distance from each other that is below a distance limit are considered co-located and called co-location pairs (e.g., a co-location pair 938 and a co-location pair 940). Said distance limit is selected in such a way that, if the distance between a reference monitor and a sensor node is lower than the distance limit, the reference monitor and the sensor node may be considered to be exposed to the same concentration of air pollutants. As a non-limiting example, the sensor node 908 and the reference monitor 928 may be the co-location pair 938, and the sensor node 916 and the reference monitor 924 may be the co-location pair 940.

In one process, the data management platform 920 identifies the co-location pairs as a sensor node and a reference monitor with a distance from each other that is within a distance limit and compares for each co-location pair the measurements from the sensor node against the measurements from the reference monitor to perform a calibration of the sensor node against the reference monitor. The result of the calibration may be a calibration profile, which may include calibration constants and a calibration model. “Calibration profile” may refer to a set of calibration constants and a calibration model. Sensor node measurements having minimal error in relation to reference monitor measurements are generated by applying the calibration constants to the sensor node measurements according to the calibration model. “Calibration constant” may refer to a whole or rational number used in place of a variable in a formula for a calibration model. A calibration model may be a parametrized mathematical or algorithmic function that may be configured to map raw sensor outputs to corrected values. It may define how to adjust the data and can be simple (e.g., linear regression) or complex (e.g., machine learning models, multivariate regression, neural networks, etc.). Complex models can have a mix of logic and multiple mathematical formulas, making up an algorithm (e.g., if condition is met, run formula 1, else run formula 2). Calibration coefficients may be the specific parameter values (e.g., numeric, Boolean, etc.) that may plug into a calibration model to perform the correction. They may be obtained through model training. As a calibration model may have one or more calibration constants, various methods may be used to determine the values for the calibration constants. In one example, calibration constants are determined by fitting a calibration model to the measurement data from a sensor and a reference monitor of a co-location pair. “Calibration model” may refer to a mathematical model for relating one or more random variables and one or more non-random variables and calibrating measurement data from a sensor to remove measurement error and bias. Examples of a calibration model may include, but are not limited to, a linear model, and the like.

In one example embodiment, a calibration model for determining calibrated measurement data may be represented by the following formula (1):

$$calibrated\_measurement = sensor measurement data * bias + offset , \qquad ( 1 )$$

where the bias and offset may include calibration constants. In another example embodiment, a calibration model for determining calibrated measurement data may be represented by the following formula (2):

$$calibrated\_measurement = sensor measurement data * a + temperature\_measurement * b + humidity\_measurement * c + d , \qquad ( 2 )$$

where a, b, c, and d may include calibration constants. In another example embodiment, a calibration model for determining calibrated measurement data may be represented by the following formula (3):

$$calibrated\_measurement = sensor measurement data \hat{} 2 * i + sensor measurement data * j + k , \qquad ( 3 )$$

where i, j, and k may include calibration constants.

The calibration constants applied to the sensor node measurements according to the calibration model may generate sensor node measurements that minimize the error between the sensor node measurements and the reference monitor measurements for the sensor node and the reference monitor belonging to a co-location pair. Each calibration profile may be stored in the storage control memory structure 922 by the data management platform 920. As a non-limiting example, the data management platform 920 may be configured to identify the co-location pair 938 and the co-location pair 940 and compare the measurements acquired by the sensor node and the reference monitor measurements in each co-location pair to calculate calibration profiles, which may include an offset coefficient and a bias coefficient as calibration coefficients, and a linear model as a calibration model.

In another process, every time a reading from a sensor node is received, the data management platform 920 may select a calibration profile stored in storage control memory structure 922 and apply the calibration profile to correct the reading acquired by the sensor node and store the reading and the corrected measurement in the storage control memory structure 922. As a non-limiting example, the measurements from the first sensor node 902, the sensor node 908, the sensor node 910, the sensor node 918 are corrected according to the calibration profile calculated from the co-location pair 938, and measurements from the sensor node 912, the sensor node 914, and the sensor node 916 are corrected according to the calibration profile calculated from the co-location pair 940.

The data management platform 920 is configured to make the measurements and the measurements stored in the storage control memory structure 922 may be sent via a data interface 942 to a data consumer 944 and a data consumer 946. In one non-limiting example, the data consumer 944 and the data consumer 946 are applications that enable the application user to display and download the air quality measurements taken by the sensor nodes at locations within the region 906 where it is desired to measure the air quality. It is to be understood that any suitable calibration(s), scaling(s), normalization(s), calibration model(s) and/or any suitable calibration coefficient(s) and/or any suitable calibration constant(s) and/or any suitable calibration profile(s) and/or any suitable model weight(s) and/or any suitable features, and/or any suitable hyperparameters, and/or the like may be referred to herein as a calibration regulator 950, which may be at least partially stored on (e.g., in any suitable memory of) and/or utilized on (e.g., by any suitable processor or controller of (e.g., for calibrating sensor data, for improving (e.g., training/testing) functionality thereof, etc.)) any suitable equipment, including, but not limited to, any suitable data management platform (e.g., platform 920, host 120, etc.), any suitable data control memory structure (e.g., structure 932, 934, etc.), any suitable data consumer (e.g., consumer 944, consumer 946, etc.), any suitable sensor or sensor node or sensor module (e.g., node 100, board 200, node 300, node 400, module 600, board 700, module 800, node 902, node 904, node 908, node 910, etc.), any suitable computing device (e.g., device 1300), and/or the like of any suitable system of the disclosure. In some embodiments, a model may be trained and tested on any suitable device, such as a laptop, a cloud server, or a cloud serverless function, and the model may be stored and used on any suitable device, such as a cloud server, cloud serverless function, or on the sensor microcontroller (e.g., on a sensor node). In some embodiments, a model may be used manually in post-processing on a laptop. A sensor or sensor component may be any suitable individual sensing element (e.g., an electrochemical cell, optical particle counter, temperature or humidity detector, etc.) that may produce raw measurements of an environmental parameter. A particular sensor or sensor component may be a target sensor or sensor of interest (“SOI”) for a calibration, normalization, scaling, and/or the like. A sensor module may be any suitable self-contained assembly that may include one or more sensors (e.g., sensor components), shared sampling hardware (e.g., fans, filters, flow paths, etc.), and/or any module-level electronics that may be used to acquire and digitize raw sensor outputs. Unless specifically designed to be otherwise, each sensor component of a sensor module may be considered to be collocated with one another. A sensor node may be any suitable monitoring device that may be configured to operate one or more sensor modules, which can be housed inside or outside of the sensor node, together with power, a microcontroller (“MCU”), communication interfaces, and/or optional onboard storage. Unless specifically designed to be otherwise, each sensor component of a sensor node (e.g., each sensor component of each sensor module of a sensor node) may be considered to be collocated with one another. Raw measurements may be the uncorrected output values that may be generated by a sensor. Calibration may be any suitable process (e.g., of a calibration regulator of an SOI) that may be configured to convert raw measurements of an SOI into calibrated measurements by applying a calibration model (e.g., an algorithm or mathematical formula) together with features, hyperparameters, and/or learned calibration coefficients or calibration model weights. A calibration may ingest raw outputs from the SOI (e.g., target sensor) and, optionally, from other collocated sensors of the same sensor node (e.g., within the same sensor module or another sensor module operated by the same sensor node) and/or from any other suitable collocated sensing capabilities (“CSCs”) that may remain collocated with the SOI as the SOI may be moved (e.g., into the field (e.g., unlike a reference monitor)), such as a temperature sensor, humidity sensor, pressure sensor, solar radiation, traffic or road proximity, and/or the like, to improve accuracy. It is to be understood that there may be a location sensor collocated with an SOI (e.g., in a sensor node) or accurate location information for the SOI may be provided to the system by an end user (e.g., by entering location coordinates through a user interface of the system), either of which may be considered a CSC (e.g., a location sensing CSC) of the SOI. Calibrated measurements may be the corrected sensor values produced by applying a calibration to raw measurements (or to normalized raw measurements). Normalization may be an adjustment of raw measurements or calibrated measurements, such as via a simple linear regression model (e.g., slope and offset), to reduce inter-sensor variability when comparing data across a group of sensors. A calibration regulator (e.g., a calibration regulator 950) may be any suitable algorithm or other suitable hardware, firmware, software, or any suitable combination thereof, that may be executable on the SOI (e.g., on an SOI's sensor node's MCU) or remotely in a data pipeline (e.g., a data management platform) or otherwise, that may be configured to apply one or more calibrations to an SOI, thereby transforming raw measurements of an SOI into calibrated measurements.

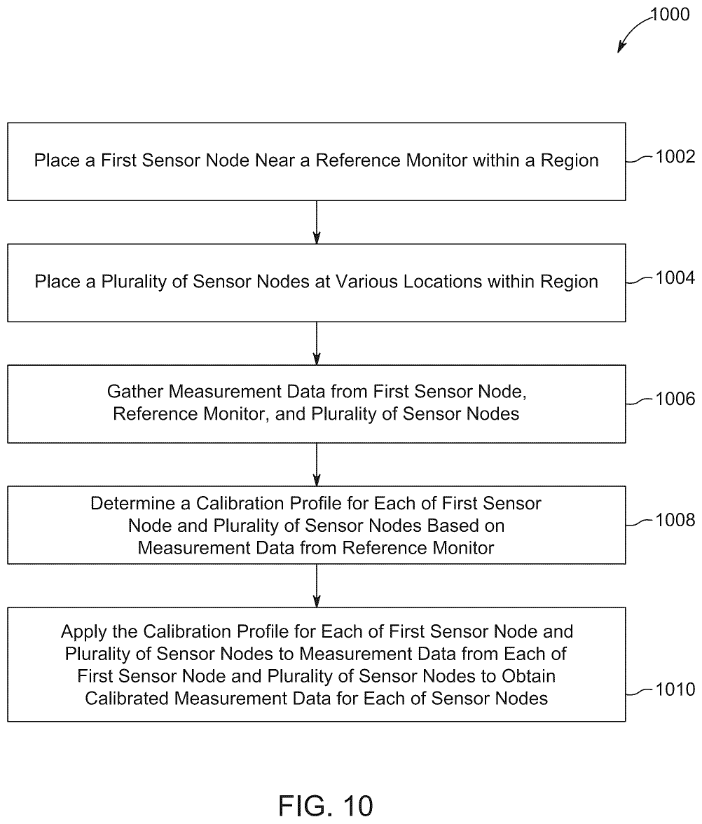

FIG. 10 illustrates an operation method 1000 in accordance with one embodiment. This method may employ a system such as that described with respect to FIG. 9.

In block 1002, operation method 1000 may place a first sensor node near a reference monitor within a region. In block 1004, operation method 1000 may place a plurality of sensor nodes at various locations within the region. In block 1006, operation method 1000 may gather measurement data from the first sensor node, the reference monitor, and the plurality of sensor nodes.

In block 1008, operation method 1000 may determine a calibration profile for each of the first sensor node and the plurality of sensor nodes based on measurement data from the reference monitor. In block 1010, operation method 1000 may apply the calibration profile for each of the first sensor node and the plurality of sensor nodes to measurement data from each of the first sensor node and the plurality of sensor nodes to obtain calibrated measurement data for each of the sensor nodes.

In some embodiments, applying the calibration profile may include wirelessly communicating a calibration profile to each of the first sensor node and the plurality of sensor nodes. In some embodiments, determining a calibration profile may include determining a calibration model for the first sensor node and determining a set of calibration constants for the first sensor node. In some embodiments, applying the calibration profile may include applying the calibration model to measurement data for the first sensor node to generate calibrated measurement data.

To ensure high accuracy, precision and reliable operation on the field, each individual sensor node may undergo thorough calibration at the factory. The modular design of the sensor node may allow for scalable and parallel calibration. Calibration of deployed sensor nodes may include transmitting a calibration profile to each of the first sensor node and the plurality of sensor nodes. This calibration profile may be transmitted wirelessly, over a cloud network configuration or by some other means. To allow city wide deployment of hundreds of sensor node, and to enable real-time reaction to air pollution events, a first sensor node may be collocated with government reference equipment for the entire duration of a monitoring project. Data from the government reference equipment may be used to calibrate data from the first sensor node, by allowing computation of bias and offset calibration constants. These computed bias and calibration constants may be applied in real-time to the plurality of sensor nodes across the entire sensor network.

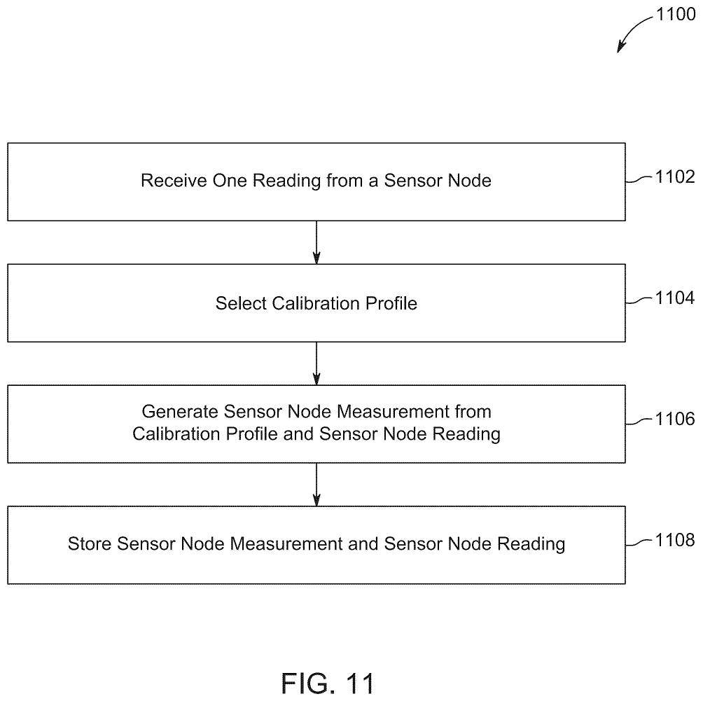

FIG. 11 illustrates the method utilized by a data management platform to correct the measurements acquired by a sensor node by applying a calibration profile in accordance with certain embodiments of the disclosed subject matter.

A calibration method 1100 includes the data management platform receiving one measurement from the sensor node (block 1102). The data management platform selects a calibration profile from the calibration profiles stored in storage media (block 1104). To select a calibration profile, the data management platform retrieves information about the node that acquired the measurement in block 1102, and information about the co-location pair that generated the calibration profile. In some embodiments the data management platform selects the calibration profile generated by the co-location pair whose reference monitor is closest to the sensor node that acquired the measurement. In other embodiments other selection criteria are used to select the calibration profile, where the selection criteria might make use of information including but not limited to land use information, meteorological information, and traffic information.

The data management platform may use the selected calibration profile to correct the sensor node measurement (block 1106). To correct the sensor node measurement, the data management platform uses the calibration constant within the calibration profile according to the calibration model within the calibration profile. Next, the data management platform may store the corrected sensor node measurement and the original sensor node measurement in storage media (block 1108).

In some embodiments, determining the calibration profile and applying the calibration profile for each of the first sensor node and the plurality of sensor nodes may be performed at a host in communication with the first sensor node, reference monitor, and plurality of sensor nodes by way of a network. This host may be a computing device such as the one illustrated in FIG. 13.

In some embodiments, applying the calibration profile for each of the first sensor node and the plurality of sensor nodes may further include communicating the calibration profile for each of the first sensor node and the plurality of sensor nodes to each of the first sensor node and the plurality of sensor nodes such that the first sensor node and the plurality of sensor nodes apply the calibration profile to generate calibrated measurement data. This communication may take place over a network as illustrated in FIG. 9.

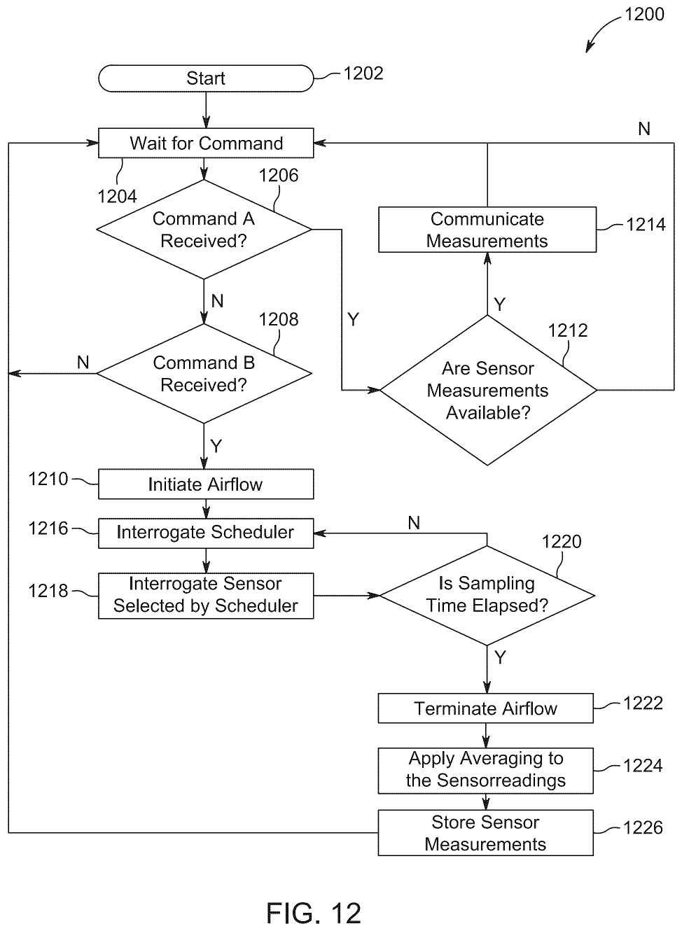

FIG. 12 illustrates a sensor module measurement method 1200 in accordance with one embodiment. The sensor module measurement method 1200 includes a start block 1202 and a state of waiting for a command (block 1204). If a command A is received (decision block 1206), then a determination is made as to whether sensor measurements are available (decision block 1212). In an embodiment, a command A is a request for sensor measurements. If the sensor measurements are available, then the measurements are communicated to the host device (such as the sensor node) via the sensor node controller 718 via serial interface (block 1214), and the method waits for an additional command (block 1204). The method also waits for an additional command if the sensor measurements are not available and communicates an error to the host device.

If a command A has not been received, then the method determines if a command B has been received (decision block 1208). In an embodiment, a command B is a request to initiate a sensor sampling procedure. If no command B has been received, then the method waits for an additional command (block 1204). Upon verification that a command B has been received, air flow is initiated in the sensor module (block 1210). Next, a scheduler is interrogated to initiate a timer and select a sensor in the sensor module (block 1216). The sensor selected by the scheduler is read and its reading is stored in a buffer (block 1218). The microcontroller checks if the sampling time has elapsed (decision block 1220). If the sampling time has not elapsed, then the scheduler is interrogated again (block 1216). If the sampling time has elapsed, then the air flow is terminated (block 1222). Next, the sensor measurements that are stored in the buffer are averaged (block 1224) and the final sensor measurements are stored (block 1226). In another embodiment, the sensor measurements that are stored in the buffer may be further processed with the aim of increasing measurement accuracy and reducing measurement noise. The method may then return to a state of waiting for a command (block 1204) and can return the final sensor measurements when requested by the host device. Command A and command B above are exemplary commands and are not limited thereto.

FIG. 13 is an example block diagram of a computing device 1300 that may incorporate embodiments of the solution. FIG. 13 is merely illustrative of a machine system to carry out aspects of the technical processes described herein and does not limit the scope of the claims. One of ordinary skill in the art would recognize other variations, modifications, and alternatives. In certain embodiments, the computing device 1300 includes a graphical user interface 1302, a data processing system 1304, a communication network 1306, communication network interface 1308, input device(s) 1310, output device(s) 1312, and the like.

As depicted in FIG. 13, the data processing system 1304 may include one or more processor(s) 1314 and a storage subsystem 1316. “Processor” may refer to any circuitry, component, chip, die, package, or module configured to receive, interpret, decode, and execute machine instructions. Examples of a processor may include, but are not limited to, a central processing unit, a general-purpose processor, an application-specific processor, a graphics processing unit (GPU), a field programmable gate array (FPGA), Application Specific Integrated Circuit (ASIC), System on a Chip (SoC), virtual processor, processor core, and the like. The processor(s) 1314 may communicate with a number of peripheral devices via a bus subsystem 1318. These peripheral devices may include input device(s) 1310, output device(s) 1312, communication network interface 1308, and the storage subsystem 1316. The storage subsystem 1316, in one embodiment, includes one or more storage devices and/or one or more memory devices. In one embodiment, storage devices may be block storage devices. “Storage device” may refer to any hardware, system, sub-system, circuit, component, module, non-volatile memory media, hard disk drive, storage array, device, or apparatus configured, programmed, designed, or engineered to store data for a period of time and retain the data in the storage device while the storage device is not using power from a power supply. Examples of storage devices include, but are not limited to, a hard disk drive, FLASH memory, MRAM memory, a solid-state storage device, Just a Bunch Of Disks (JBOD), Just a Bunch Of Flash (JBOF), an external hard disk, an internal hard disk, and the like. “Memory” may refer to any hardware, circuit, component, module, logic, device, or apparatus configured, programmed, designed, arranged, or engineered to retain data. Certain types of memory require availability of a constant power source to store and retain the data. Other types of memory retain and/or store the data when a power source is unavailable. “Block storage device” may refer to a storage device configured to interface with external hosts, servers, components, controllers, and the like according to a block protocol. This means that under normal operating conditions data stored on the storage media of the block storage device is accessible by storage command that operate on one or more data blocks. Access to data of the block storage device is not available under normal operating conditions in quantities smaller than that defined for a data block. In other words, normal operations with a block storage device do not permit access to data at a word, byte, or bit level.

In one embodiment, the storage subsystem 1316 includes a volatile memory 1320 and a non-volatile memory 1322. The volatile memory 1320 and/or the non-volatile memory 1322 may store computer-executable instructions that alone or together form logic 1324 that when applied to, and executed by, the processor(s) 1314 implement embodiments of the processes disclosed herein. “Volatile memory” may refer to a shorthand name for volatile memory media. In certain embodiments, volatile memory may refer to the volatile memory media and the logic, controllers, processor(s), state machine(s), and/or other periphery circuits that manage the volatile memory media and provide access to the volatile memory media. “Volatile memory media” may refer to any hardware, device, component, element, or circuit configured to maintain an alterable physical characteristic used to represent a binary value of zero or one for which the alterable physical characteristic reverts to a default state that no longer represents the binary value when a primary power source is removed or unless a primary power source is used to refresh the represented binary value. Examples of volatile memory media include but are not limited to dynamic random-access memory (DRAM), static random-access memory (SRAM), double data rate random-access memory (DDR RAM) or other random-access solid-state memory.

While the volatile memory media is referred to herein as “memory media,” in various embodiments, the volatile memory media may more generally be referred to as volatile memory.

In certain embodiments, data stored in volatile memory media is addressable at a byte level which means that the data in the volatile memory media is organized into bytes (8 bits) of data that each have a unique address, such as a logical address. “Non-volatile memory” may refer to a shorthand name for non-volatile memory media. In certain embodiments, non-volatile memory media may refer to the non-volatile memory media and the logic, controllers, processor(s), state machine(s), and/or other periphery circuits that manage the non-volatile memory media and provide access to the non-volatile memory media.

“Non-volatile memory media” may refer to any hardware, device, component, element, or circuit configured to maintain an alterable physical characteristic used to represent a binary value of zero or one after a primary power source is removed. Examples of the alterable physical characteristic include, but are not limited to, a threshold voltage for a transistor, an electrical resistance level of a memory cell, a current level through a memory cell, a magnetic pole orientation, a spin-transfer torque, and the like. The alterable physical characteristic is such that, once set, does not change so much when a primary power source for the non-volatile memory media is unavailable the alterable physical characteristic can be measured, detected, or sensed, when the binary value is read, retrieved, or sensed. Said another way, non-volatile memory media is a storage media configured such that data stored on the non-volatile memory media is retrievable after a power source for the non-volatile memory media is removed and then restored.

Examples of non-volatile memory media include but are not limited to: ReRAM, Memristor memory, programmable metallization cell memory, phase-change memory (PCM, PCME, PRAM, PCRAM, ovonic unified memory, chalcogenide RAM, or C-RAM), NAND flash memory (e.g., 2D NAND flash memory, 3D NAND flash memory), NOR flash memory, nano random access memory (nano RAM or NRAM), nanocrystal wire-based memory, silicon-oxide based sub-10 nanometer process memory, graphene memory, Silicon-Oxide-Nitride-Oxide-Silicon (SONOS), programmable metallization cell (PMC), conductive-bridging RAM (CBRAM), magneto-resistive RAM (MRAM), magnetic storage media (e.g., hard disk, tape), optical storage media, or the like. While the non-volatile memory media is referred to herein as “memory media,” in various embodiments, the non-volatile memory media may more generally be referred to as non-volatile memory. Because non-volatile memory media is capable of storing data when a power supply is removed, the non-volatile memory media may also be referred to as a recording media, non-volatile recording media, storage media, storage, non-volatile memory, volatile memory medium, non-volatile storage medium, non-volatile storage, or the like.

In certain embodiments, data stored in non-volatile memory media is addressable at a block level which means that the data in the non-volatile memory media is organized into data blocks that each have a unique logical address (e.g., LBA). In other embodiments, data stored in non-volatile memory media is addressable at a byte level which means that the data in the non-volatile memory media is organized into bytes (8 bits) of data that each have a unique address, such as a logical address. One example of byte addressable non-volatile memory media is storage class memory (SCM). “Logic” may refer to machine memory circuits, non-transitory machine readable media, and/or circuitry which by way of its material and/or material-energy configuration includes control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories including software and thereby forming configurations of matter). “Partition identifier” may refer to any identifier for a logical partition or physical partition.

The input device(s) 1310 may include devices and mechanisms for inputting information to the data processing system 1304. These may include a keyboard, a keypad, a touch screen incorporated into the graphical user interface 1302, audio input devices such as voice recognition systems, microphones, and other types of input devices. In various embodiments, the input device(s) 1310 may be embodied as a computer mouse, a trackball, a track pad, a joystick, wireless remote, drawing tablet, voice command system, eye tracking system, and the like. The input device(s) 1310 may typically allow a user to select objects, icons, control areas, text and the like that appear on a graphical user interface 1302 via a command such as a click of a button or the like.

The output device(s) 1312 may include devices and mechanisms for outputting information from the data processing system 1304. These may include the graphical user interface 1302, speakers, printers, infrared LEDs, and so on. In certain embodiments, the graphical user interface 1302 is coupled to the bus subsystem 1318 directly by way of a wired connection. In other embodiments, the graphical user interface 1302 couples to the data processing system 1304 by way of the communication network interface 1308. For example, the graphical user interface 1302 may include a command line interface on a separate computing device 1300 such as desktop, server, or mobile device.

The communication network interface 1308 provides an interface to communication networks (e.g., communication network 1306) and devices external to the data processing system 1304. The communication network interface 1308 may serve as an interface for receiving data from and transmitting data to other systems. Embodiments of the communication network interface 1308 may include an Ethernet interface, a modem (telephone, satellite, cable, ISDN), (asynchronous) digital subscriber line (DSL), FireWire, USB, a wireless communication interface such as Bluetooth or WiFi, a near field communication wireless interface, a cellular interface, and the like.

The communication network interface 1308 may be coupled to the communication network 1306 via an antenna, a cable, or the like. In some embodiments, the communication network interface 1308 may be physically integrated on a circuit board of the data processing system 1304, or in some cases may be implemented in software or firmware, such as “soft modems”, or the like.

The computing device 1300 may include logic that enables communications over a network using protocols such as HTTP, TCP/IP, RTP/RTSP, IPX, UDP and the like.

Volatile memory 1320 and the non-volatile memory 1322 are examples of tangible storage media configured to store computer readable data and instructions to implement various embodiments of the processes described herein. “Storage media” may refer to any physical media organized and configured to store one or more bits of data. In one embodiment, storage media may refer to physical storage cells and/or memory cells used in volatile memory media. In another embodiment, storage media may refer to physical storage cells and/or memory cells used in non-volatile memory media. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), battery-backed volatile memories, networked storage devices, and the like. The volatile memory 1320 and the non-volatile memory 1322 may be configured to store the basic programming and data constructs that provide the functionality of the disclosed processes and other embodiments thereof that fall within the scope of the present disclosure.

Logic 1324 that implements one or more parts of embodiments of the solution may be stored in the volatile memory 1320 and/or the non-volatile memory 1322. Logic 1324 may be read from the volatile memory 1320 and/or non-volatile memory 1322 and executed by the processor(s) 1314. The volatile memory 1320 and the non-volatile memory 1322 may also provide a repository for storing data used by the logic 1324.

The volatile memory 1320 and the non-volatile memory 1322 may include a number of memories including a main random-access memory (RAM) for storage of instructions and data during program execution and a read only memory (ROM) in which read-only non-transitory instructions are stored. The volatile memory 1320 and the non-volatile memory 1322 may include a file storage subsystem providing persistent (non-volatile) storage for program and data files. The volatile memory 1320 and the non-volatile memory 1322 may include removable storage systems, such as removable flash memory.

The bus subsystem 1318 provides a mechanism for enabling the various components and subsystems of data processing system 1304 communicate with each other as intended. Although the communication network interface 1308 is depicted schematically as a single bus, some embodiments of the bus subsystem 1318 may utilize multiple distinct busses.

Computing device 1300 may be any suitable device, including, but not limited to, a smartphone, a desktop computer, a laptop computer, a rack-mounted computer system, a computer server, a sensor node, or a tablet computer device. Computing device 1300 may be implemented as a collection of multiple networked computing devices. Further, the computing device 1300 may include any suitable operating system logic (not illustrated).

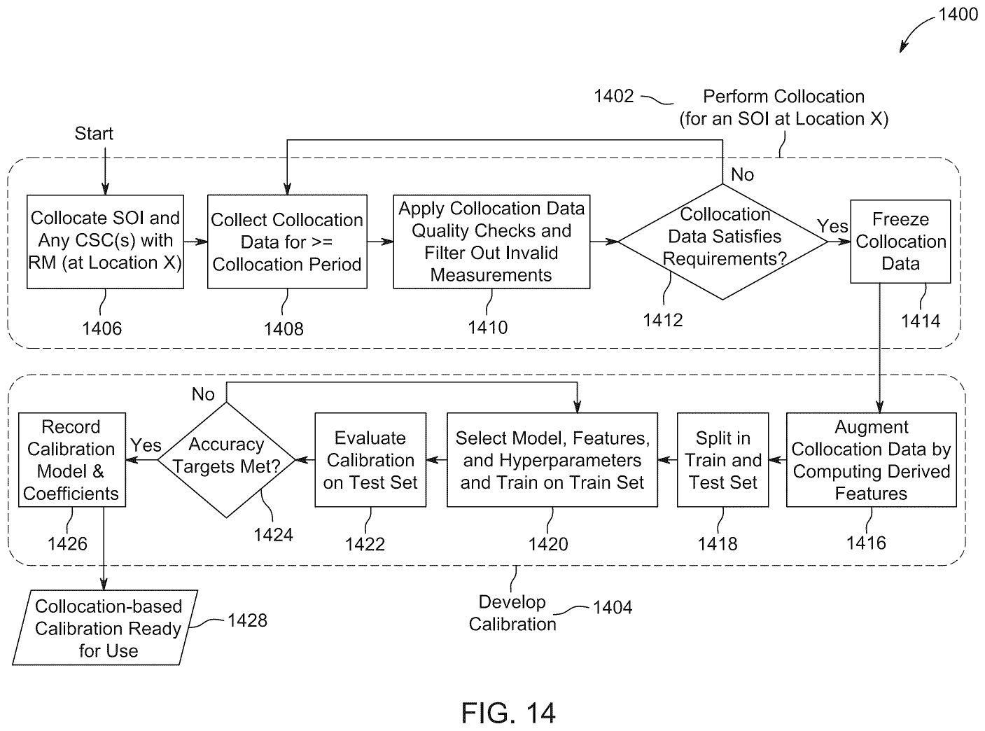

### FIGS. 14-23

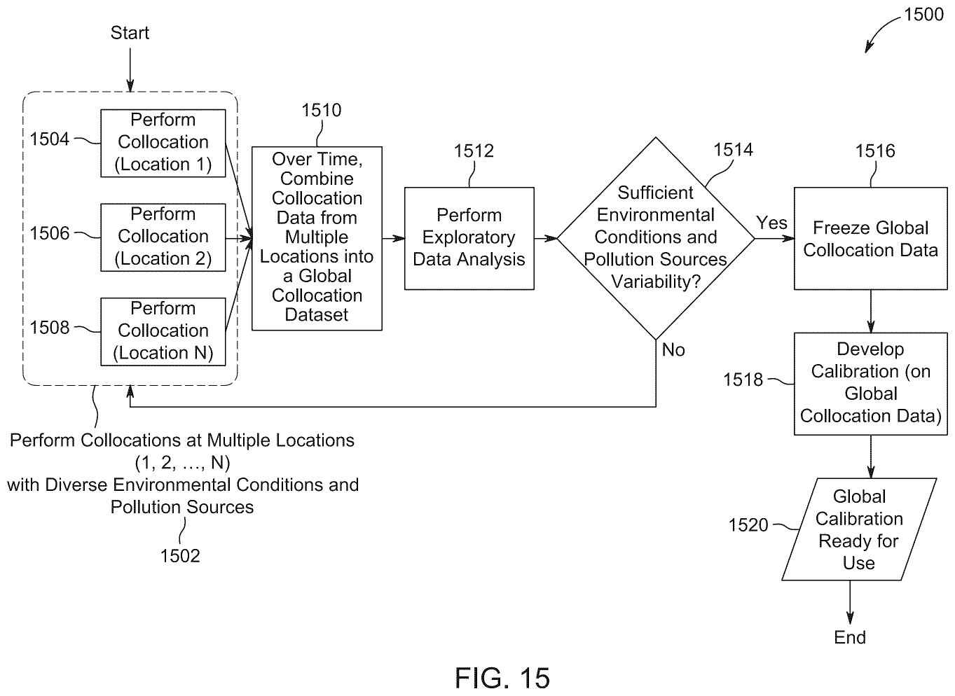

Calibration of particulate matter sensors is important for many reasons. Air quality sensor nodes increasingly use low-cost particulate matter (“PM”) sensors (e.g., optical particle counter (“OPC”) sensors, nephelometers, or other low-cost sensors) to measure particulate matter mass concentrations. Such sensors, however, may not directly measure PM mass concentration. Instead, they may estimate PM mass concentration (e.g., by counting the number of particles across several size bins). To accurately estimate PM mass concentrations, knowledge of particle density and other physical parameters related to chemical composition may be essential. Factory calibrations may input an assumed particulate composition-related conversion factor into each sensor. However, this assumption might not accurately reflect real-world ambient conditions, leading to potential measurement discrepancies. In addition, some PM sensor embodiments may not be able to detect all particle sizes, but present detection cutoffs for particles smaller than a smallest detectable particle size and for particles larger than a largest detectable particle size, where beyond the cutoffs the detection efficiency of the PM sensor may fall sharply. Thus, some PM sensor embodiments may rely on assumption of particle size distribution in order to output a mass concentration measurement. Assumption of particle size distribution may refer to how the PM sensor may handle particle sizes that lie outside its detection limits (i.e., beyond the smallest and largest detectable cutoffs). In other words, because the PM sensor cannot detect particles smaller than its minimum-size cutoff or larger than its maximum-size cutoff, it may assume some distribution for those unmeasured ranges to convert counts of detected particles into a total mass concentration. Within the size bins that the PM sensor can detect, it may use actual measured data. Beyond those cutoffs, the PM sensor may rely on an assumed distribution to estimate the particle-mass contribution from sizes it cannot see. To address this, a model (e.g., a multiple linear regression model) may be trained on collocation data collected from diverse environments across the globe. This training may help develop a generalized calibration profile, which may be referred to as a global PM calibration, which may aim to minimize such errors (see, e.g., process 1500 of FIG. 15). Further corrections can be achieved through collocation with a reference monitor at specific project sites, allowing for the development of collocation-based calibrations that may be fine-tuned to local pollutant and environmental conditions, thus enhancing measurement accuracy (see, e.g., process 1400 of FIG. 14).

Calibration of gas sensors (e.g., electrochemical cell sensors (“ECS”)) measuring various gaseous pollutants, including, but not limited to, nitrogen dioxide (“NO₂”), nitric oxide (“NO”), carbon monoxide (“CO”), ozone (“O₃”), hydrogen sulfide (“H₂S”), sulfur dioxide (“SO₂”), ammonia (“NH₃”), and other gaseous pollutants, can be important for many reasons. Sensor nodes may employ gas sensors for measuring NO₂, NO, CO, O₃, H₂S, SO₂, NH₃, and/or any other gaseous pollutant concentrations. These sensors may operate by reacting with the target gas to produce an electric current that is indicative of the target gas levels. However, sensor performance can be compromised by environmental interferences, such as fluctuations in temperature and humidity, and/or cross-sensitivity to other gas-phase pollutants. To mitigate these effects, a model (e.g., an ensemble-based regression machine learning model, such as LightGBM) can be utilized. This model may be trained on collocation data gathered from diverse locations across the globe to create a global calibration (e.g., a global NO₂ calibration), thereby significantly reducing the impact of environmental interferences on sensor readings. Additionally, specific collocation-based calibrations can be developed for each sensor through targeted collocation studies, which may further enhance the sensor's ability to correct for environmental interferences and align its output more closely with that of reference instruments.

Calibration of aethalometers measuring black carbon (“BC”) may be important for many reasons. An aethalometer may be a specialized instrument that may be used to measure the concentration of BC in the air. It may operate by drawing air through a filter, where particles may be collected. A light beam may then be directed through the filter, and the instrument may measure the attenuation of light caused by the particles deposited on the filter. The degree of light attenuation may be proportional to the amount of black carbon present. Measurements carried out with different light frequencies can be compared to understand the source of black carbon (e.g., biomass burning or fossil fuel combustion). By continuously monitoring the light attenuation and airflow, the aethalometer may provide real-time data on black carbon concentrations. Rapid changes of temperature can correlate with shifts in the baseline output of certain aethalometers (e.g., their output when exposed to 0 parts per billion (“PPB”) of BC). Calibration can help to reduce the error resulting from the baseline shifts. This may be especially important when aethalometers are stationed outdoors for continuous monitoring, as they are likely to experience rapid temperature changes.

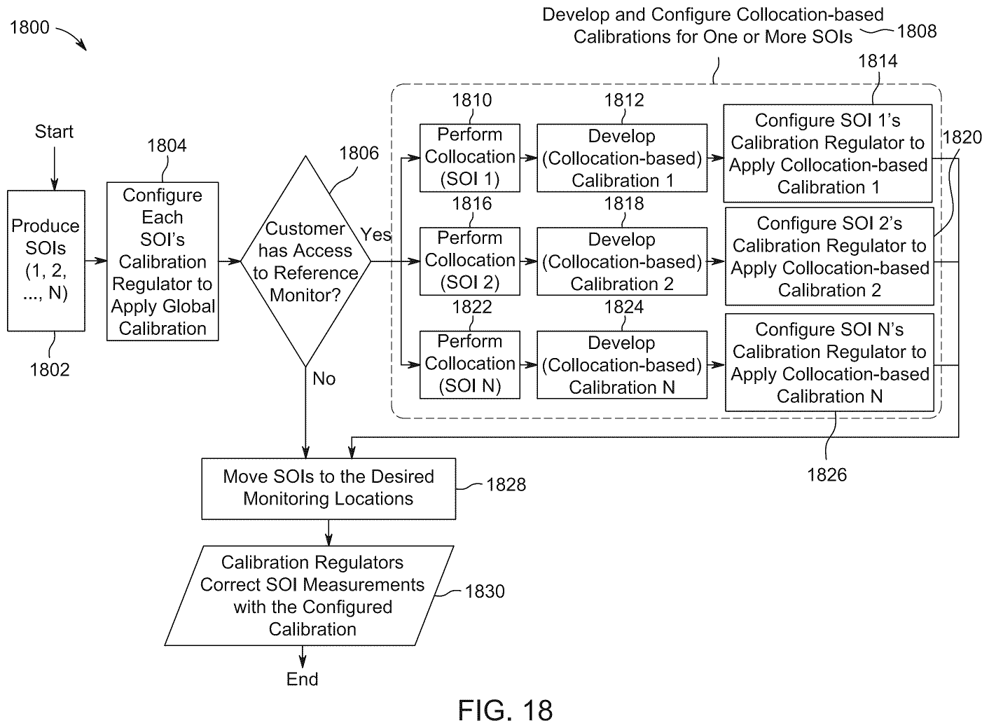

Air quality sensors may require calibration to deliver accurate and reliable data. Calibration may align sensor outputs with established reference standards, which may be essential for effective air quality monitoring and decision-making. Different calibration schemes of increased sophistication may be carried out to increase sensor accuracy. The proper approach may be selected based on the data use case, striking a balance between cost/complexity and accuracy. As a non-limiting example, consider two different calibration approaches: global calibration and collocation-based calibration. Global calibrations for PM and gaseous pollutants (e.g., NO₂) may be developed from a large historical dataset of collocated measurements, where one or more sensors may be deployed side-by-side with one or more reference monitors across one or more diverse environments and/or one or more pollutant levels to ensure they are exposed to identical or substantially identical atmospheric conditions across such diverse environments and/or such pollutant levels, and can be applied to newly produced sensors without the need for further testing. These global calibrations can significantly improve sensor accuracy compared to raw measurements and can enable immediate sensor deployment even when a local reference monitor is unavailable. Raw measurements may refer to the uncorrected sensor outputs before any calibration is applied. For example, for a PM sensor, it may be the direct mass estimate that the sensor reports without any adjustment. As another example, for a gas sensor, it may be the unadjusted voltage or current reading from the working electrode, converted to a concentration estimate only by the sensor's manufacturer-provided sensitivity factor. Therefore, raw measurements may be the sensor's native, out-of-the-box values (e.g., raw PM_{2.5} mass concentration [μg/m³] or raw NO₂ concentration [ppb]), such as the raw data produced before it may be processed by a global and/or custom (e.g., collocated) calibration. For higher accuracy requirements, sensors can undergo custom collocation-based calibration by being collocated with reference monitors for an extended period, such as four weeks. This can allow for calibrations that may account for local environmental and pollution conditions and may enable calculation of device-specific accuracy metrics, such as R² and RMSE, providing transparency and traceability to the calibrated results. By working with customers, it may be possible to choose between different calibration approaches, or a combination of different calibration approaches. For example, as shown in FIG. 18, a process 1800 may include an operation 1802, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of sensor nodes or SOIs (e.g., SOIs 1-N) may be produced or otherwise obtained. At operation 1804, each calibration regulator of each one of the SOIs may be configured to apply global calibration (e.g., global calibration as may be developed by any suitable process, such as by process 1500 of FIG. 15). At operation 1806, based on any suitable information (e.g., based on the onboarding of operation 1802 or otherwise (e.g., if the customer has access to one or more reference monitors), collocation may be recommended to the customer and process 1800 may advance from operation 1806 to operation 1808 where collocation-based calibrations may be developed and configured for each SOI (see, e.g., operations 1810-1826, which may also be described by process 1400 of FIG. 14), before advancing to operation 1828 (e.g., for SOI 1, operation 1810 may perform collocation with a reference monitor, then operation 1812 may develop collocation-based calibration 1, and then operation 1814 may configure a calibration regulator for SOI 1 to apply collocation-based calibration 1; for SOI 2, operation 1816 may perform collocation with a reference monitor, then operation 1818 may develop collocation-based calibration 2, and then operation 1820 may configure a calibration regulator for SOI 2 to apply collocation-based calibration 2; and, for SOI N, operation 1822 may perform collocation with a reference monitor, then operation 1824 may develop collocation-based calibration N, and then operation 1826 may configure a calibration regulator for SOI N to apply collocation-based calibration N, etc.).

Otherwise, process 1800 may advance from operation 1806 to operation 1828, where the SOIs may be moved to the desired monitoring location (e.g., to the appropriate positions in the field as desired by the customer for end use). After operation 1828, process 1800 may advance to operation 1830, where the calibration regulator(s) of each SOI may correct measurements of the SOI with the applicable configured calibration(s) (e.g., global calibration and/or collocation-based calibration of the calibration regulator).

Calibration may involve collecting training and testing data from sensors, often alongside reference instruments, in environments where the true pollutant concentration may be known. This can occur during periods when sensors may be collocated with reference instruments in ambient air (see, e.g., operation 1402 (e.g., operations 1406-1414) of process 1400 of FIG. 14) or during factory characterization where sensors may be exposed to controlled environments with known pollutant concentrations.

A calibration model may then be selected to represent the relationship between the sensor's raw data and the true pollutant concentration. This model can be based on physics, machine learning, or a combination of both. It may take various input features, including raw sensor measurements, transformations of these measurements, data from other sensors in the same environment (e.g., temperature, relative humidity, etc.), derived features created through mathematical transformations and feature engineering techniques, and/or any other available information regarding the site or region where the sensor is deployed (e.g., population density, nearest road type and distance, road length within a distance buffer by road type, traffic, weather, etc.). In addition to or as an alternative to temperature (“T”) and/or relative humidity (“RH”), a calibration model may be configured to incorporate any suitable variables, including, but not limited to, one or more of the following variables (e.g., provided there is a way to measure or obtain the variable(s) alongside the target sensor (e.g., sensor of interest (“SOI”)))
1. Barometric Pressure (“P”) (e.g., pressure can affect gas sensor diffusion rates or aerosol density; an onboard compact barometric pressure sensor or an external weather API linked to the sensor's GPS location may be used to capture such data; and the system may be configured to include pressure as a linear or polynomial feature (e.g., P or P²) to correct for signal drift at high/low altitudes or rapid weather changes);
2. Wind Speed and Wind Direction (e.g., turbulence and plume dispersion can affect local concentration and sometimes sensor response (e.g., if sampling flow rates vary); an onsite anemometer combined with the sensor node, or a nearby meteorological station or weather model via API, may be used to capture such data; and the system may be configured to use wind speed/direction as inputs in a model to account for dilution effects or spurts of high concentration from particular directions);
3. Particulate Composition Proxies (e.g., Coarse vs. Fine Ratio) (e.g., the PM sensor response may change depending on whether particles are mostly dust (e.g., coarse) or combustion (e.g., fine), as may be derived from raw size-bin data (e.g., ratio=(PM₁₀−PM_{2.5})/PM_{2.5}) and/or number-concentration ratios (PM_{2.5} count/PM₁ count); this may be directly computed from simultaneous Pm sensor size-bin outputs; and the system may be configured to use these ratios in calibration to identify dust events, correct for non-linear scattering under dusty conditions (e.g., as in the Texas dust-sensitive features example), and/or the like);
4. Time of Day and Solar Radiation (e.g., diurnal cycles can introduce systematic biases (e.g., ozone interferences in NO₂ sensors at midday); timestamps in the data log and/or, optionally, geolocated solar irradiance from a local weather station or solar radiation sensor combined with the sensor node operating the sensor may be used to capture such data; and the system may be configured to encode time-of-day as sine/cosine variables and/or include solar irradiance as a feature for ozone cross-sensitivity corrections);
5. Traffic and/or Road Proximity (e.g., NO₂ spikes may often correlate with traffic patterns, and distance to nearest road or traffic volume can help discriminate local peaks; any suitable geographic information system (“GIS”) lookup for road type/distance, or live traffic API if available, may be used to capture such data; and the system may be configured to include “distance to major road” or “expected traffic index” or any other suitable data points as a static or time-varying feature in the model);
6. Population Density and/or Land Use Index (e.g., urban vs. rural environment may influence typical pollutant mixes (e.g., high PM_{2.5} from cooking, secondary aerosols, etc.); GIS land-use maps linked to the sensor's coordinates may be used to capture such data; and the system may be configured to use as a categorical or continuous feature so the model can learn baseline offsets by land-use type (e.g., industrial zone vs. residential, etc.)); and/or
7. Altimeter/Altitude (e.g., high altitudes may mean different baseline gas partial pressures and aerosol densities); a small onboard or otherwise collocated barometric sensor plus known site elevation may be used to capture such data; and the system may be configured to include altitude (or barometric pressure) in the regression to correct for baseline shifts in sensor response).
   It is to be understood that there may be a location sensor collocated with a sensor component or SOI (e.g., in a sensor node) or accurate location information for the sensor component may be provided to the system by an end user (e.g., by entering location coordinates through a user interface of the system), either of which may be considered a collocated sensing capability (“CSC”) of the sensor component. One, some, or all of the above extra variables may become additional input features of a calibration model (e.g., whether linear regression or ensemble). During training, the model may be configured to learn coefficients or tree splits that may capture how each environmental or contextual variable systematically skews the raw sensor response. Then, at runtime, each new measurement may be paired with contemporaneous readings of T, RH, pressure, and/or the like, computed through the same feature transformations, and fed into the model. The model output may be a corrected pollutant concentration that accounts for all considered variables. Mathematical transformations (e.g., derived features that may capture non-linearities, temporal dynamics, and/or event-driven anomalies in the raw data, including, but not limited to, temperature polynomial baseline (e.g., by T_{baseline}=c₁(T²−25²)+c₂(T−25)+c₃ (e.g., captures non-linear shifts in the sensor baseline as temperature deviates from 25° C. (e.g., models a quadratic-like drift with T that gas sensors may exhibit))), time-dependent RH baseline (e.g., exponential filter (e.g., RH_{baseline}t=ΔRH×δRhConst+R_{baseline}t−1×exp(−Δt/τ) (e.g., models gas sensors' baseline shifts that may be caused by sudden humidity changes while attenuating the influence of older events (e.g., effectively approximating a high-pass filter in time)))), dust-sensitive squared differences (e.g., for PM sensors (e.g., (PM₁₀−PM_{2.5})², (PM_{2.5}−PM₁)²) (e.g., emphasizes large size-bin disparities during dust events (e.g., these terms may improve performance by correcting PM_{2.5} underestimation)))), ratio features (e.g., PM_{2.5}/PM_{1.0}, PM_{1.0}/PM₁₀) (e.g., captures relative shifts in particle-size distribution (e.g., distinguishing coarse dust from fine combustion), interaction terms (e.g., PMraw_{2.5}×RH, v_{Gas}×T) (e.g., handles situations where two variables jointly distort the sensor signal more than each alone (e.g., high humidity+high temperature causing extra baseline drift)), rolling window statistics (e.g., 14-day rolling average of raw measurements, 20th/80th percentiles of recent measurements, days since deployment (e.g., helps the model learn and compensate for gradual drift over time and normalize out slow seasonal trends)), and/or the like) may be chosen in any suitable manner, including, but not limited to, physical insight (e.g., knowing that humidity affects gas sensors baselines with a characteristic time constant suggests using an exponential filter for RH), empirical testing (e.g., collocation case studies (e.g., during dust events) may reveal which transformations, such as squared PM differences, significantly improve R\hat{ }2), and/or the like. The model may use these inputs to output inferred pollutant concentrations, which may be adjusted according to the model's coefficients. Features may be added or removed based on whether they improve performance on held-out data without overfitting, for example, during a cross-validation process. A calibration model may be a formula or algorithm (e.g., possibly based on physics, statistics, and/or machine learning, etc.) that may define how input features (e.g., raw sensor readings, temperature, humidity, contextual metadata, and/or the like) may be mathematically combined to estimate the true pollutant concentration. The calibration coefficients (or model parameters) may be the values or parameters learned during the training phase, typically using data from a reference monitor (“RM”). These coefficients are not adjusted during normal sensor operation but instead may usually be fixed once the model is trained. In some embodiments, during deployment, the raw sensor data and other input features may be fed into the calibration model, the pre-trained calibration coefficients may be applied within the model, and the model may produce the inferred pollutant concentration as output. The coefficients may be the fixed values (e.g., numbers, Boolean value (e.g., True, False), etc.) plugged into the model, which together may translate input features into corrected output values. They are usually not being re-adjusted during routine measurement.

The model may be trained on the collected training data to optimize its coefficients, minimizing the difference between the model's inferred pollutant concentrations and the known true concentrations. Testing data may be used to validate the model's accuracy and ensure it is not overfitted, employing techniques like cross-validation.

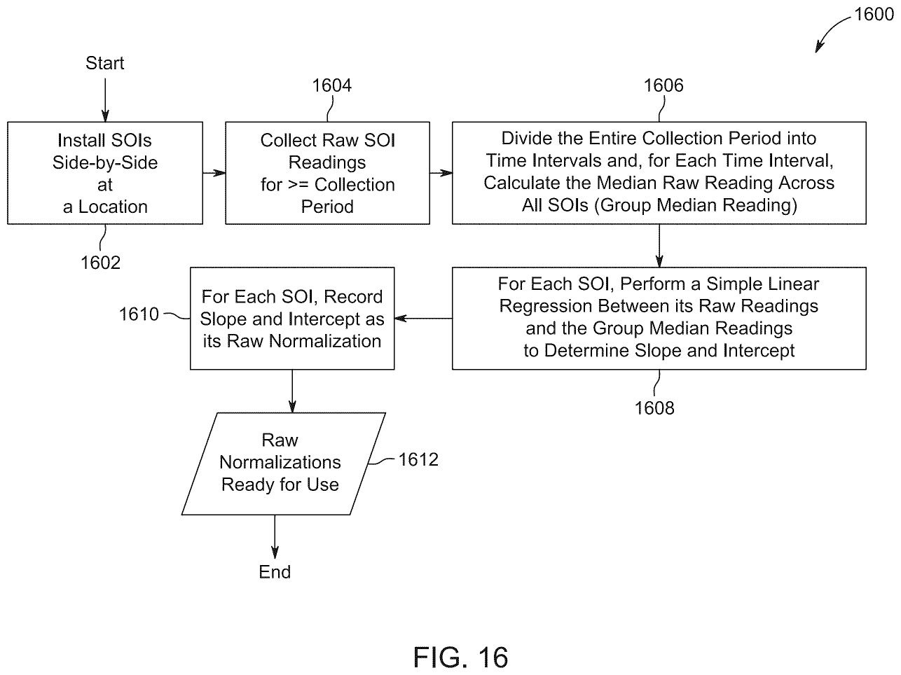

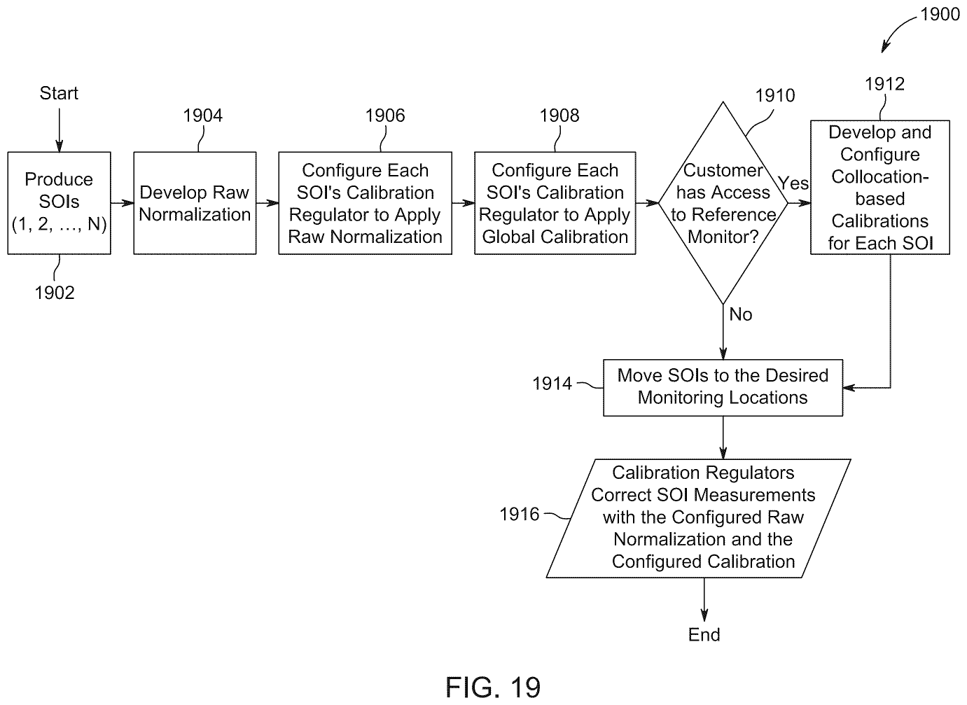

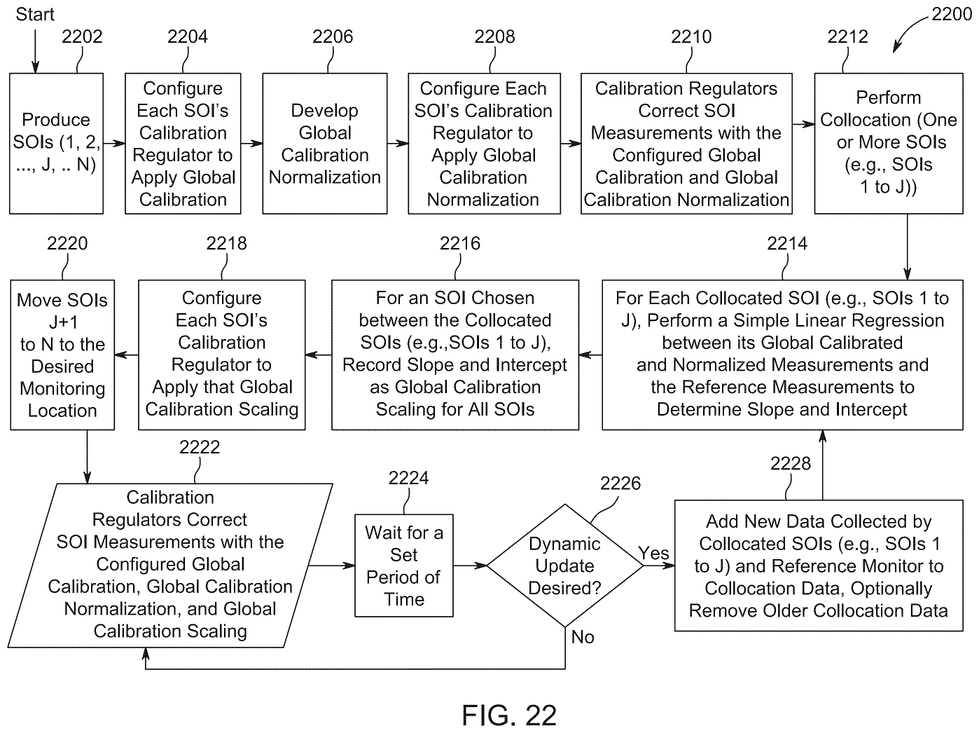

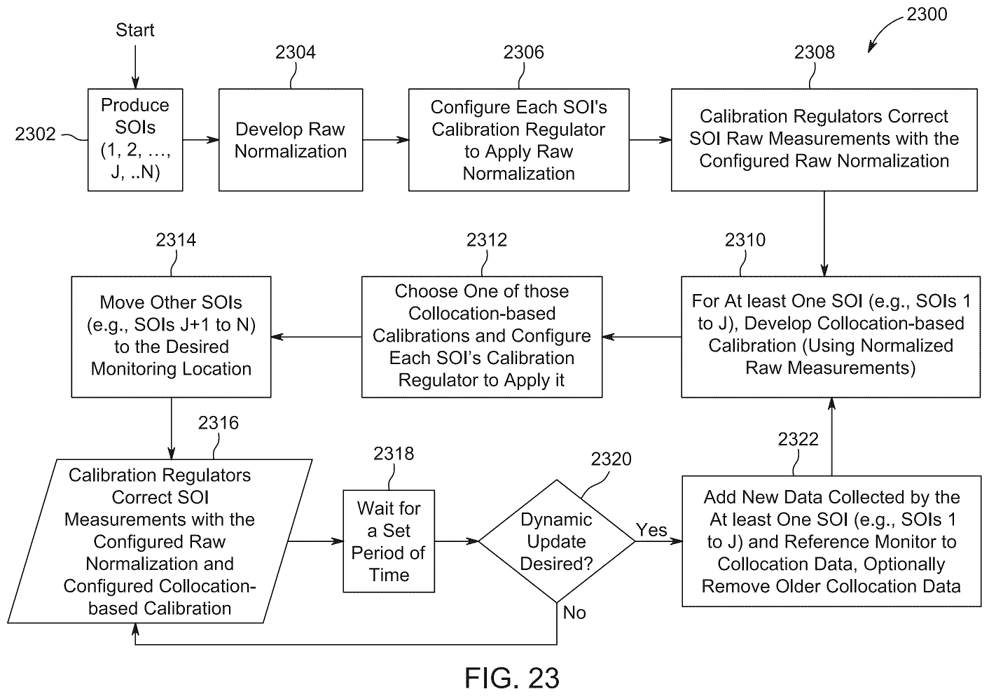

Once trained, the calibration model, which may be referred to as the calibration or calibration profile or calibration regulator (e.g., regulator 950), can be applied in real-world scenarios to data captured by sensors deployed in the field to provide calibrated measurements, thereby offering more accurate pollutant concentrations than raw sensor data. Raw measurements or raw sensor data may refer to the uncorrected sensor outputs before any calibration is applied. For example, for an optical particle counter, it may be the direct count or mass estimate that the sensor reports without any adjustment. As another example, for an electrochemical gas sensor, it may be the unadjusted voltage or current reading from the working electrode, converted to a concentration estimate only by the sensor's manufacturer-provided sensitivity factor. Therefore, raw measurements may be the sensor's native, out-of-the-box values (e.g., raw PM_{2.5} mass concentration [μg/m³] or raw NO₂ concentration [ppb]) produced before passing through a global and/or custom calibration algorithm. In some embodiments, pre-calibration of a sensor device may be performed (e.g., during manufacturing of the device and/or prior to providing the device to an end user (e.g., at operation 1804)) by applying a global calibration (e.g., enabling a model and associated characteristics to be accessible to the device) that may be developed from extensive historical collocation datasets that may be collected across diverse environmental and pollutant conditions. For example, applying a calibration may generally refer to ensuring that every raw sensor measurement is run through that calibration before being reported as a final (e.g., calibrated) value. In practice, there may be various approaches, including, the following:
1. On-Device (Firmware) Implementation (e.g., the calibration (e.g., a serialized LightGBM tree file or a set of regression coefficients) may be embedded in the sensor's firmware at the factory; when the sensor measures raw data (e.g., voltages, counts, pollutant concentration estimates, etc.), its onboard microcontroller may compute all derived features (e.g., baseline corrections, interactions, etc.) and then evaluate the global calibration in real time; the firmware may be configured to output the calibrated pollutant concentration immediately from the device's own processing; and/or the calibration can, optionally, be updated by remote firmware upgrades);
2. Cloud-Based Implementation (e.g., the sensor node may send raw sensor measurements (e.g., of the SOI) (e.g., along with any environmental data (e.g., T, RH, etc.)) to a cloud server; a cloud API or microservice may be configured to apply the calibration to those inputs (e.g., computing derived features server-side); and/or the server may then return a calibrated pollutant concentration to the user through a data dashboard, API, data storage, or the like); and/or.
3. Post-processing (e.g., all the raw measurements plus any additional data may be logged during monitoring; and an analyst or computer code may calculate derived features and correct all the data applying the calibration in bulk and on demand).
   Either approach may satisfy the phrase “applying a calibration.” One way to refer to applying a global calibration may be processing each raw sensor measurement (and its associated environmental/contextual inputs) through a fixed, pre-trained global calibration function, whether in embedded firmware or via a cloud service, so that the output may be a calibrated pollutant concentration rather than the uncorrected raw value. This may allow one or more sensor devices to be deployed immediately, even in the absence of access to a local reference monitor. In this context, immediately may mean that, once the sensor is turned on in its target deployment location, it can start producing calibrated pollutant readings right away, without waiting for any on-site collocation or additional tuning. In other words: no field delay (e.g., the sensor may not need to remain next to a reference for days or weeks before its data becomes “valid”); and/or deploy-and-go (e.g., the customer can install the sensor and begin streaming data within minutes or hours, rather than waiting for a local calibration period). Therefore, immediately may refer to the absence of any waiting period for on-site or project-specific calibration; the device may be effectively “plug-and-play” once pre-calibrated with a global calibration. In scenarios where higher measurement precision may be required, collocation-based calibration may be recommended. This process may involve placing one or more sensors (e.g., sensor nodes, SOIs, etc.) in close proximity to one or more high-accuracy reference monitors for any suitable period of time (e.g., at least four weeks) to ensure exposure of each sensor of a sensor device/reference monitor collocated set to the same or substantially the same atmospheric conditions (see, e.g., operation 1402 of process 1400 of FIG. 14). In various embodiments, certain collocation techniques may be followed. For example, for particulate matter collocation-based calibration, at least one sensor may be collocated with a reference monitor for at least a certain collection period of time (e.g., at least two weeks, at least four weeks, etc.), where collocation of at least three sensors with a reference monitor may be useful to ensure redundancy and mitigate the impact of any device-specific anomalies or failures. For example, for PM_{2.5} calibration (e.g., calibration for sensors configured to sense PM with a diameter of 2.5 μm or less), the collocation period should include at least one day where the average mass concentration exceeds a particular threshold (e.g., 25 μg/m³). In such embodiments, the threshold (e.g., 25 μg/m³ for PM_{2.5}) may be evaluated based on the reference monitor's 24-hour average, not the low-cost sensor's raw reading. In other words, the system may determine that “a day exceeded 25 μg/m³” when the collocated reference instrument reports an average above that threshold. Such a threshold can be adjusted. The exact threshold may vary by region or by pollutant. It may be selected to ensure that during the collocation period, the sensor has experienced a wide enough concentration range (e.g., both low and moderately high levels) so that the calibration model can learn to correct across that spectrum. If there is no variability or range in pollutant concentration, there is risk of overfitting (e.g., overoptimizing for those unchanging conditions and performing poorly when the field conditions change). The specific threshold (e.g., 25 μg/m³ for PM_{2.5}) may be chosen empirically. It may be a low enough range that can be consistently achieved in relatively clean locations without having to extend the test indefinitely, and it may be high enough to reduce the risk of overfitting. For PM₁₀, the period may include at least one day exceeding a particular threshold (e.g., 50 μg/m³). If such a threshold is not met within a particular collection period (e.g., four weeks), the collocation may be extended in one-week increments until the threshold condition may be satisfied. For gas sensor calibration, in some embodiments, all sensors may be deployed at the collocation site to capture sensor-to-sensor variability and ensure consistent exposure. In some other embodiments (see, e.g., process 1600 of FIG. 16, operation 1904 of process 1900 of FIG. 19, etc.), all sensors may first be deployed side-by-side in an outdoor environment, such as a parking lot, without a reference monitor, for at least two weeks or any other suitable threshold period. This configuration may enable normalization of sensor outputs (e.g., adjusting each sensor's raw output to align with the mean output of the group or a selected representative sensor (e.g., using a linear correction of the form: corrected_output=raw_output×a+b, where a and b may be determined from the data collected during the two-week normalization period). Subsequently (see, e.g., operation 1912 of process 1900 of FIG. 19), at least one sensor may be collocated with a reference monitor for another threshold period (e.g., four weeks) to enable collocation-based calibration. In yet some other embodiments, normalization may be carried out at the factory, either in outdoor conditions or within a controlled characterization chamber. For gas sensor calibration, a collocation period may be configured to include at least one day with average gas concentrations exceeding a specific threshold (e.g., 20 ppb for NO, 15 ppb for NO₂, 0.75 ppm for CO, 35 ppb for O₃, etc.). In such cases, temperature and relative humidity during collocation may be controlled to be representative of the expected deployment environment, ideally covering the 25th to 75th percentile ranges for these parameters. In moderate environments, typical acceptable ranges may be 15-30° C. for temperature and 40-80% for relative humidity. Whenever possible, collocation sites may be selected to provide exposure to a wide range of pollutant concentrations and environmental variability. If immediate deployment is necessary, pre-calibrated sensors may be installed initially, with collocation conducted in parallel or at a later stage to enable retrospective calibration and enhancement of the collected data. Retrospective calibration may refer to a situation where, after pre-calibrated sensors have been deployed and collected a period of raw (or globally calibrated) data, the system may later perform a collocation, develop a collocation-based calibration, and then apply the collocation-based calibration backwards to all data collected since initial deployment. As an example, to enable retrospective calibration, the following may occur: (1) at time T₀, deploy sensor with a global calibration only and then start collecting data; (2) at time T₁ (e.g., weeks or months or any suitable period of time later), perform a collocation (e.g., place the same sensor next to a reference for four weeks); (3) use the collocation dataset (e.g., from T₁ to T₁+4 weeks or any other suitable period of time) to train a collocation-based calibration model; and (4) apply the resulting collocation-based calibration retroactively to all sensor data from T₀ onward (e.g., because the system may have the sensor's raw (or globally calibrated) outputs from T₀ to T₁, the system may be configured to pass them through the newly trained collocation-based calibration to produce “corrected” values for that entire period). Data obtained during collocation may be used either to validate the applicability of existing global calibration profiles or to develop collocation-based calibrations (see, e.g., operation 1808 of process 1800 of FIG. 18). Calibration performance may be evaluated using predefined statistical criteria, including, but not limited to, R², root mean square error (“RMSE”), mean absolute error (“MAE”), slope, offset, any suitable uncertainty metrics, and/or the like, to determine whether the global, or the collocation-based calibration ought to be applied. If collocation is not feasible, organizations may rely on global calibrations, with an understanding of potential sources of residual measurement error. To support ongoing data quality, maintaining at least one sensor permanently collocated with a reference monitor may be recommended (see, e.g., process 2200 of FIG. 22, process 2300 of FIG. 23, etc.). Data from this sensor may be used to evaluate long-term performance and determine whether periodic recalibration is warranted. In some embodiments, calibration decisions may be guided by evaluating sensor performance using a combination of any suitable techniques, including the coefficient of determination (R²) and an error metric such as mean absolute error (MAE) or root mean square error (RMSE). R² may indicate how well the sensor's calibrated output tracks the variations in pollutant concentrations observed by a reference monitor, while RMSE may quantify the absolute magnitude of error between the two. By jointly interpreting these metrics, operators may be enabled to distinguish (e.g., automatically) between different types of calibration outcomes. Any suitable structured decision matrix may be employed, such as one where sensors may be classified into one of the following four performance categories based on sensed data:
(1) High R²/Low RMSE: The sensor performs well and can be considered accurate and reliable for operational use, no further calibration is needed;
(2) High R²/High RMSE: The sensor captures trends correctly but exhibits a significant bias or scaling error, calibration or calibration update to be carried out to improve absolute accuracy;
(3) Low R²/Low RMSE: The pollutant range is likely too narrow to meaningfully assess performance, the collocation period ought to be extended to include a period with a broader concentration range; and
(4) Low R²/High RMSE: The sensor is inaccurate and cannot produce useful data, it may be defective or unsuitable for the measurement environment, or a more sophisticated calibration approach such as layered calibration ought to be attempted.
   In practice, whether R² or RMSE is considered “High” or “Low” may depends on the pollutant, the sensor type, and/or the project' accuracy requirements (e.g., there is no single universal cutoff). Generally, R² thresholds may tend to be higher for PM sensors (e.g., around 0.70-0.80) and lower for gas sensors (e.g., around 0.50-0.60). As RMSE carries units, its threshold may vary substantially by pollutant. One approach may be to take the concentration at the “Good/Moderate” air quality index (“AQI”) breakpoint and multiply it by a factor less than 1 (e.g., 0.5), as setting RMSE below that value may ensure the error remains small enough to correctly classify the AQI category. Such a decision matrix may be used in automated or manual workflows to approve or reject calibrations, flag sensors for rework, and/or decide when to reinitiate collocation and update calibrations. In some embodiments, these thresholds may be visualized on a two-dimensional chart plotting R² against MAE or RMSE, thereby enabling quick identification of acceptable vs. problematic calibrations. In some embodiments, the system may use these performance zones to determine whether to apply a global or collocation-based calibration profile. When results for a sensor with pre-applied global calibration fall in the “High R²/High RMSE” quadrant, a custom collocation-based calibration may be preferable to reduce absolute error. If the sensor falls in the “Low R²/High RMSE” zone, the unit may be flagged for removal or return, or a more sophisticated calibration approach, such as layered calibration, can be attempted.

### Global Calibration—Overview

To simplify the calibration process, especially in scenarios where access to reference monitors may be limited or non-existent, sensors may be calibrated out-of-the box using global calibrations (see, e.g., operation 1804 of process 1800 of FIG. 18, etc.). These global calibrations may be derived from extensive collocation datasets that may be collected across various environmental conditions worldwide (see, e.g., process 1500 of FIG. 15). The global calibrations may significantly enhance the accuracy of raw sensor data by providing a reliable baseline correction that can be applied universally, regardless of the specific sensor unit or deployment location. Global calibration may provide several benefits, including, but not limited to, immediate deployment readiness (e.g., sensors can be deployed quickly without the need for onsite calibration, which may be ideal for projects with tight timelines or budget constraints), enhanced data accuracy (e.g., even without local calibration, global calibrations can substantially improve the sensor's raw data accuracy, making them suitable for general air quality assessments), reduced calibration effort (e.g., organizations can avoid the complexities and costs that may be associated with conducting local calibrations), and/or the like.

### Custom Collocation-Based Calibration—Overview

Although global calibration may provide a solid baseline accuracy, performing collocation-based calibration at a project site or elsewhere may further refine sensor performance by allowing for environment-specific corrections (see, e.g., operation 1808 of process 1800 of FIG. 18, etc.). In some embodiments, this method may involve placing air quality sensors in close proximity to a reference monitor (e.g., for several weeks or for any other suitable period of time), to directly compare and adjust the sensor's readings against the reference (see, e.g., process 1400 of FIG. 14). This site-specific calibration may allow for any suitable results, including, but not limited to, tailored accuracy improvements (e.g., adjustments that account for local environmental variables, such as specific pollutant concentrations and microclimatic conditions, which may not be fully covered by global calibrations), quantifiable performance metrics (e.g., collocation may provide clear performance indicators for each sensor (e.g., improved R² and/or reduced RMSE values), which may be crucial for validating the calibration's effectiveness), sensor-to-sensor variation reduction (e.g., collocation-based calibration may allow for the individual testing and tuning of each sensor, enabling adjustments tailored to specific sensor characteristics, whereby this process may help minimize variations between devices, ensuring that each sensor delivers consistent and accurate measurements, even among units with inherent manufacturing discrepancies), and/or the like.

### Layered Calibration—Overview

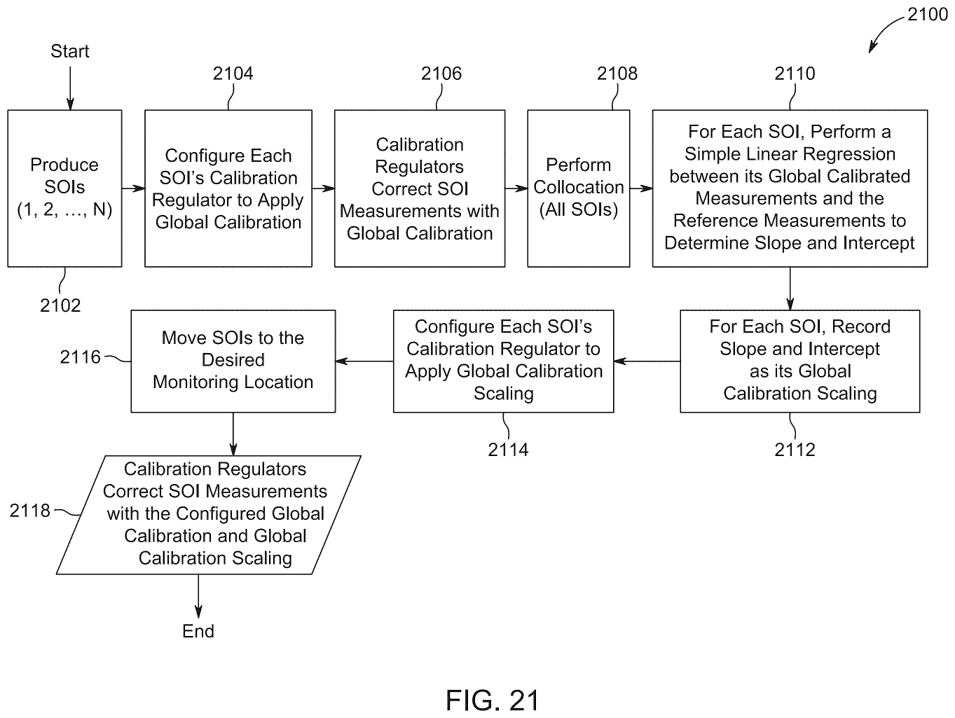

Layered calibration may integrate both global and collocation-based calibration, and, in some cases, sensor normalization, to achieve optimal sensor accuracy (see, e.g., process 2100 of FIG. 21, process 2200 of FIG. 22, process 2300 of FIG. 23, etc.). In some embodiments, this technique may begin with a global calibration that may be configured to convert raw sensor data into global calibrated measurements. Following this, a more targeted collocation-based calibration may further refine global calibrated measurements using specific data from the deployment site. This multi-stage process may enhance sensor accuracy by addressing both broad and localized environmental factors. Global calibrations may rely on a far larger dataset, which may be collected over months or years and/or may cover a wide range of pollutant levels and/or environmental conditions. As so much training data may be available (e.g., using data collected from all sensor nodes being used worldwide or even just a subset thereof), global models can afford to be relatively complex (e.g., LightGBM with many features) without overfitting to a narrow set of circumstances. By contrast, collocation-based calibrations may use a much smaller, site-specific dataset that often may not span the full spectrum of concentrations and/or environmental variability. To avoid overfitting, these local models typically may follow simpler, physics-informed forms and/or may use fewer features, which also may keep the operational burden of developing a collocation calibration manageable. In a hybrid (e.g., layered) approach, the system may first apply the global calibration to capture a sensor's general behavior under diverse conditions. Then, the system may use the global-calibrated output as an input feature for the collocation-based calibration, which may fine-tune the correction to match site-specific patterns. In other words, a global model's prediction may become one of the inputs to a collocation model, thereby allowing the collocation-based calibration to focus solely on residual, location-specific biases. Because global models may be trained on a very large dataset, they may not be re-trained during the collocation stage. Instead, the collocation model may be trained separately on local reference data, using the global output as a baseline feature, so that each stage may learn from its own dataset without altering the global model's weights. Although, it is possible to reverse the order (e.g., perform collocation first and then roll those data into a larger global model) or train both simultaneously in a unified framework, although the sequential global-then-local workflow may be more effective as it may leverage extensive historical data first and then may home in on local nuances without risking overfitting. Advantages of such a layered calibration approach may include, but are not limited to, uniform baseline accuracy (e.g., a process may start with a global calibration that standardizes sensor outputs, ensuring all sensors begin with a uniform level of accuracy regardless of their individual characteristics), site-specific refinement (e.g., a process may further refine sensor accuracy with collocation-based calibration that incorporates local environmental data, tailoring sensor outputs to specific conditions of the deployment site), flexibility in implementation (e.g., a process can involve setting up temporary collocations with reference monitors or utilizing a cluster of sensors to establish a local normalization baseline, offering flexibility based on available resources and project needs), robustness against environmental variations (e.g., a process may include the combination of global and localized calibration that may make sensors robust against both widespread environmental variations and specific local anomalies), efficiency in calibration transfer (e.g., a process may allow for the calibration of a single sensor in a collocation setup, with the refined calibration parameters then transferred to other sensors in the network, streamlining the process and reducing the need for multiple collocations of different sensors), dynamic calibration updates (e.g., an initially collocated sensor can be periodically re-evaluated to update the calibration, which can then be transferred to other sensors in the field, ensuring continued accuracy over time and adapting to environmental changes), scalability (e.g., a process may not only maximize the accuracy and reliability of air quality measurements but may also provide a scalable solution that can be adapted to varying project scales and environmental conditions), and/or the like.

### Calibration of Black Carbon Monitors Based on Temperature Change Rate—Overview

A calibration method may address the challenge of baseline shifts in black carbon monitors that may be caused by rapid temperature changes. These shifts can lead to inaccurate measurements, compromising the reliability of environmental data, especially when black carbon monitors are deployed in a stationary setting outdoors. To mitigate this, a calibration method may be employed that characterizes the baseline shift due to the temperature rate of change in each black carbon monitor during production and applies a regression model to account for monitor-specific shifts. This method may enhance the performance and reliability of black carbon monitors across various deployment scenarios and environmental conditions. Advantages of the approach may include, but are not limited to, enhanced accuracy (e.g., by accounting for the effects of temperature change rates, the method may ensure that black carbon concentration measurements are accurate and reliable, even in environments or deployment scenarios with rapid temperature changes), real-time adjustments (e.g., the calibration can be applied directly within the device firmware or through cloud-based software, allowing for real-time adjustments and continuous accuracy, and, by recalculating derived outputs such as smoothed black carbon concentration and source apportionment estimates, the method may eliminate the need for retroactive data quality control (“QC”) and processing), improved decision-making (e.g., accurate and real-time data from calibrated black carbon monitors may support better decision-making in environmental health assessments and air quality studies), and/or the like.

### Global Calibration

A broad dataset derived from any suitable number (e.g., millions) of collocated measurements across any suitable number (e.g., hundreds, thousands, etc.) of sensors that may be deployed in any suitable locations (e.g., diverse cities in different climatic regions) side by side with reference monitors may be used to train one or more statistical models (e.g., a global calibration model (see, e.g., process 1500 of FIG. 15, operation 1804 of process 1800 of FIG. 18, etc.)). The trained statistical models (i.e., the global calibrations) may be optimized to calibrate the raw readings of sensors into calibrated measurements, which may minimize the error of such calibrated measurements compared to measurements from reference monitors known to be accurate in measuring pollutant concentrations. In some embodiments, one global calibration model may be trained per pollutant. In some embodiments, new global calibration for the same pollutant can be developed periodically that perform better, for example, using new collocation data and research findings.

In some embodiments, data from the sensor being calibrated may be augmented by integration with additional environmental or pollution sensor data, such as, for example, by incorporating simultaneous measurements of temperature (T) and relative humidity (RH) sensors exposed to the same air sample as the sensor being calibrated (e.g., all additional sensor data (e.g., T, RH, etc.) may also be captured during the accumulation of data to be used for training a global model). Data collected during collocation-based calibration may then also be added to the larger dataset used for training global calibration. Additional data may also be sensed by any suitable CSCs (e.g., the sensors operated by the sensor module that is operating the sensor being calibrated, or be available from third party sources at the envisioned deployment location). For example, if the system is using traffic data from a third party data source, the system may require the data be available at collocation sites for global model training, and at the field deployment locations for global model usage. If a field deployment location does not have traffic data, then the global calibration that includes it might not be used.

In some embodiments, additional derived features may be calculated from readings of the sensor being calibrated, readings of additional environmental sensors exposed to the same sample of air, physical principles, and/or the like. These derived features may include time dependent or interaction terms to capture non-linearities in a sensor's response to the environment.

In some instances, the baseline of a sensor of NO₂, NO, CO, O₃, or any other suitable gas sensors (e.g., their output when exposed to clean air) may be impacted by relative humidity changes. In some embodiments, a time-dependent relative humidity baseline derived feature may be calculated to compensate for these non-linear effects on sensor baseline. This feature may use a mathematical model based on the concept of a high-pass filter, with two key coefficients: deltaRhConst, which may scale the impact of humidity changes, and tau, a time constant that may control the response rate. Humidity levels and time intervals between measurements may be monitored. When a humidity change is detected, a new baseline value may be calculated using the following formula (4):

$$New Baseline = Δ Humidity \cdot deltaRhConst + Previous Baseline \cdot e \hat{} ( - \frac{Δ Time}{tau} ) . \qquad ( 4 )$$

In some embodiments, such a formula or algorithm may work by attenuating the influence of older humidity changes using an exponential decay function based on the elapsed time since each event. Each ΔHumidity term may be multiplied by an attenuation factor of e\hat{ }(−ΔTime/tau), so that older events may contribute progressively less to the current baseline. This may allow the baseline to adapt dynamically to recent environmental conditions while filtering out long-term drift effects. In other embodiments, the current value of the baseline may be computed using a truncated time-weighted summation of historical humidity changes. This may be particularly suitable when only a fixed amount of historical data can be stored due to memory constraints. Such an algorithm may be configured to calculate the baseline using the following formula (5):

$${Baseline}_{n} = deltaRhConst \times {\sum }_{i} [ ( {Rh}_{i} - {Rh}_{i - 1} ) \times exp ( - ( {t}_{n} - {t}_{i} ) / tau ) ] , \qquad ( 5 )$$

where the sum may be taken over a finite, recent subset of past measurements (e.g., the last 3,000 samples), and each humidity difference may be scaled by an exponential decay factor representing its temporal distance from the current time (e.g., this new baseline may be one of the derived features for the model). By discarding events whose contribution falls below a minimum attenuation threshold (e.g., 1%), the algorithm may ensure that the stored sample history is always sufficient to compute the baseline to the desired accuracy. This embodiment makes the computation tractable and efficient for embedded devices with limited memory, while preserving the essential dynamic behavior of the full high-pass filter model. A time-dependent RH baseline (e.g., the high-pass filter formulation) may be only one derived feature among many in the global model. A complete global calibration model (e.g., LightGBM) may be configured to combine dozens of features (e.g., voltage-to-concentration conversions, T_baseline, RH_baseline, rolling statistics, dust metrics, interaction terms, etc.). An RH baseline filter may be just a single feature that may capture how humidity transients may shift the sensor's zero point (e.g., it is not the entire calibration; it is one piece that the global algorithm may be configured to use alongside many others). In some embodiments, the baseline of electrochemical cell sensors may also be affected by temperature. To account for this, a temperature baseline correction term may be introduced into the calibration model. In some embodiments, the temperature baseline may be modeled as a second-order polynomial of the form of the following formula (6):

$$Basline\_Temp = {c}_{1} \times {T}^{2} + {c}_{2} \times T + {c}_{3} , \qquad ( 6 )$$

where T may be the ambient temperature and c₁-c₃ may be learned coefficients. This formulation may capture the non-linear relationship between temperature and the sensor's baseline output. In other embodiments, the temperature correction may use alternative functional forms, including, but not limited to, higher-order polynomials, splines, exponential functions, or logarithmic transformations, depending on the observed behavior of the sensor and the complexity desired to capture temperature-induced bias. These temperature baseline features may be used alongside a humidity baseline correction within a multi-feature calibration model to improve overall performance across a range of environmental conditions. For example, this derived feature may be used within a calibration model to separate transient baseline shifts caused by humidity from a sensor signal caused by the presence of the target pollutant (e.g., the pollutant detectable by the target sensor or SOI (e.g., SOI pollutant)).

In some instances, sensors may show poor correlation with reference measurements during certain periods. For example, during periods with high dust concentrations, sensors might under-report PM2.5 levels compared to reference monitors, while in other periods, they might over-report. In some embodiments, various calculated features, such as the squared differences between raw measured PM10 and PM2.5 mass concentration, and PM2.5 and PM1 mass concentration, may be particularly useful. These features may capture the non-linear relationships and variances in sensor responses during dust events. The squared differences may emphasize larger discrepancies between sensor readings, making the model more sensitive to periods when dust significantly impacts the measurements. In addition to squared differences, other mathematical variations of the relationship between raw PM10, PM2.5, and PM1 measurements can be valuable. These may include the ratios between PM2.5, PM10, and PM1 mass and number concentration measurements. Such ratios can highlight relative changes in particle concentrations during dust events. Furthermore, mathematical transformations of these ratios and differences, such as square root, exponential, square, cube, logarithm, and/or the like, may capture complex patterns in the data, enhancing the model's ability to correct for dust influences. In some embodiments, two dust-sensitive features may be defined by the following formulas (7) and (8):

$$pm 10 \_pm 2 \_ 5 \_diff \_squared = {( pm 10 - pm 2 \_ 5 )}^{2} ; and \qquad ( 7 )$$

$$pm 2 \_ 5 \_pm 1 \_diff \_squared = {( pm 2 \_ 5 - pm 1 )}^{2} \qquad ( 8 )$$

where pm10, pm2_5, and pm1 may be the raw PM10, PM2.5, and PM1 mass concentration measurements from the sensor in micrograms per cubic meter (μg/m³). These squared difference terms may emphasize large disparities between size bins, which may be indicative of coarse-mode dust contributions. In one exercise, these features were evaluated in collocations in Lubbock and El Paso, Texas, in April. During high-dust events, it was observed that the raw PM2.5 sensor data underestimated reference measurements, while PM10 readings remained elevated. When these derived features were included in calibration models, performance metrics such as R² and RMSE improved significantly. For example, a multivariate regression model of the form of the following formula (9):

$$pm 2 \_ 5 \_calibrated = {c}_{1} \times pm 10 + {c}_{2} \times pm 10 \_pm2 \_ 5 \_diff {\_squared}^{2} + {c}_{3} \times pm 2 \_ 5 \_pm 1 \_diff {\_squared}^{2} + {c}_{4} \times RH + {c}_{5} \qquad ( 9 )$$

outperformed standard models, particularly in dust conditions. Here, RH may represent relative humidity, and c₁-c₅ may be regression coefficients fit to the data. The similarity of model coefficients across both Texas sites where the test was conducted suggests these features may be generalizable to other dust-influenced environments.

The global calibrations may be optimized for their ability to be applied to new sensors, thereby resulting in accurate data without having to characterize or collocate the new sensors with a reference monitor, thereby enabling accurate out-of-the-box operation of new sensors (e.g., global calibrations may be developed using historical collocation data from select sensors, and they can be applied on any newly produced sensor without needing to do further testing and adjustments on the sensor at the factory, such as measuring zero-air bias and a single gas point in a chamber). Alternatively, a different approach may use minimal characterization of the new sensors that may be carried out inexpensively at the factory along with a global calibration to ensure accurate out-of-the-box operation. Therefore, in some embodiments, once the system has a global calibration, a brand-new sensor can be shipped “out of the box” and immediately apply that global model without any extra factory operations (e.g., the model may already account for typical manufacturing variability), while, in other embodiments, if the system wants even tighter out-of-the-box accuracy, the system may be configured to perform a lightweight factory characterization (e.g., measuring zero-air bias and a single gas point) to compute a small adjustment, then still apply the global model (e.g., optionally, the system may either rely on the global model alone, or may combine it with a brief factory check and tune-up to slightly improve initial accuracy).

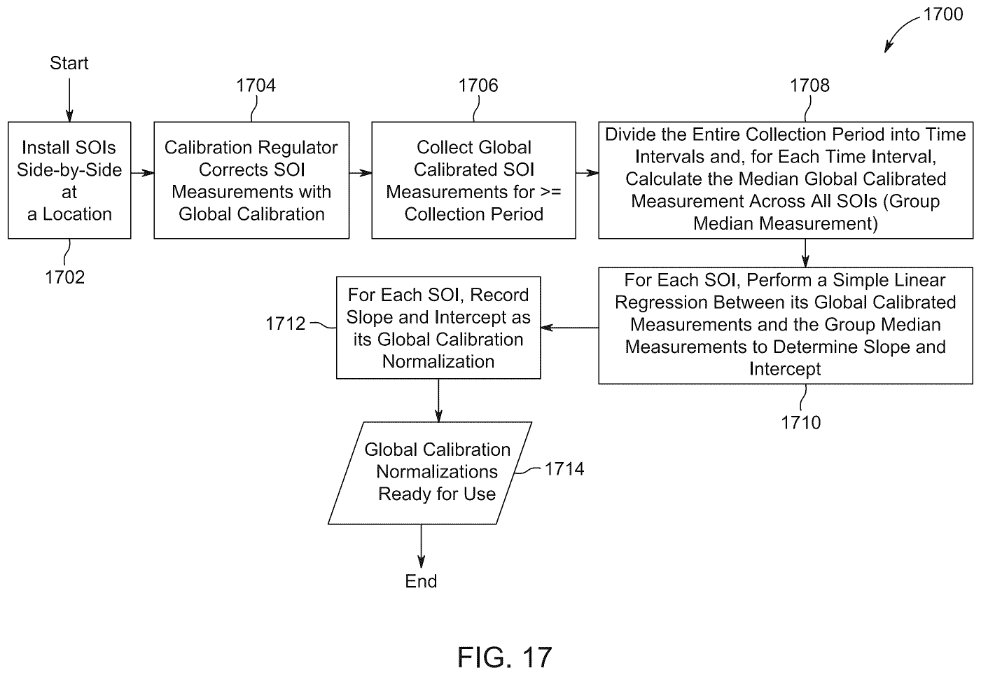

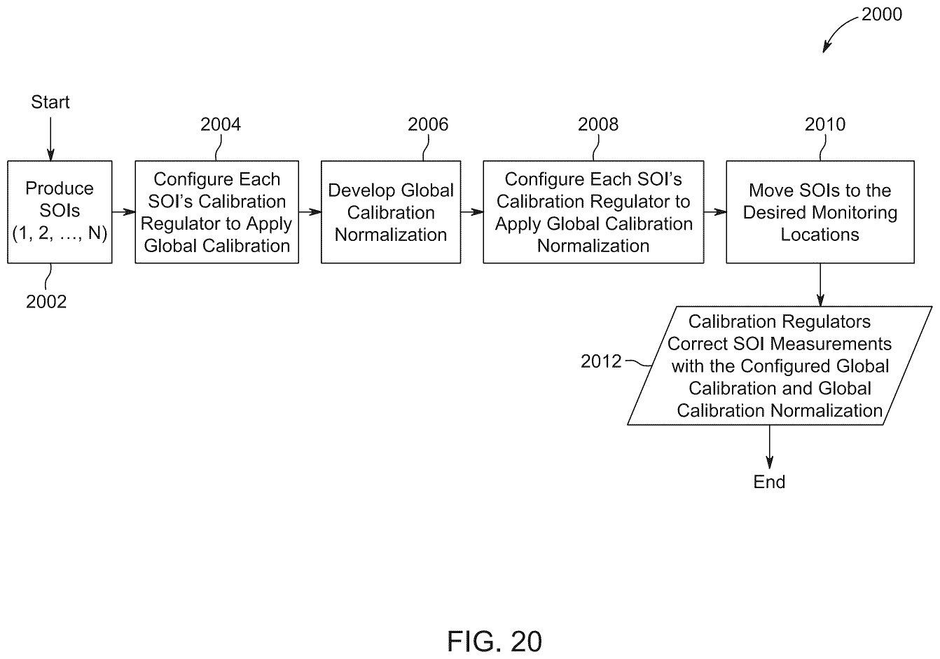

In some embodiments, a decision-tree-based learning model, such as LightGBM, can be utilized for its efficacy in handling non-linear responses of sensors to environmental factors. In some embodiments, a global calibration model for NO₂ may be implemented using any suitable decision-tree-based ensemble algorithm, such as LightGBM, that may be trained on a large collocation dataset, which may include data collection spanning multiple regions and/or multiple seasons. A goal of such a model may be to correct gas sensor output for non-linear and/or environment-dependent effects, such as those caused by changes in temperature, humidity, interfering gases, and/or the like. Such a model may use both measured sensor outputs and a wide array of derived features. The raw inputs may include, but are not limited to, the following:
(i) vGas and vAux (e.g., the electrochemical cell sensor working and auxiliary electrode measured voltages (e.g., in millivolts));
(ii) ecsSensitivity (e.g., a sensitivity factor for the electrochemical cell sensor, which may be based on manufacturer characterization of the sensor, and may be expressed in nA/ppb, where, for example, this sensitivity factor may be temperature corrected (e.g., by using a lookup function));
(iii) Ambient temperature (e.g., ambient temperature (T) in degrees Celsius); and/or
(iv) Relative humidity (e.g., relative humidity (RH) in percentage).
   From these raw variables, the model may be configured to compute several intermediate features, including, but not limited to, the following:
(i) dew point estimate (e.g., dewPoint, which may be estimated from RH and T measurements using any suitable technique (e.g., using the Magnus formula approximation), which may be expressed in degrees Celsius);
(ii) voltage-to-concentration conversions, which may use the following formulas:

$$no 2 VGas\_ppb = ( vGas \times {10}^{6} / 499 ) / escSensitivity ; \qquad ( 10 )$$

$$no 2 VAux\_ppb = ( vAux \times {10}^{6} / 499 ) / escSensitivity ; and / or \qquad ( 11 )$$

$$no 2 \_ppb = no 2 VGas\_ppb - no 2 VAux\_ppb , \qquad ( 12 )$$

where, in such expressions, (vGas×10⁶/499) and (vAux×10⁶/499) may convert the working and auxiliary electrode voltages into electrical current in nanoamperes, based on the design of the potentiostat readout circuit (e.g., assuming a feedback resistor of 499 kΩ), where this conversion may reflect the measured electrochemical current corresponding to gas interaction (vGas) or to the auxiliary/reference behavior (vAux), and where the result may then be divided by the ecsSensitivity parameter, which may be a sensitivity coefficient (e.g., in nA/ppb) provided by the sensor manufacturer, where the resulting values, no2VGas_ppb and no2VAux_ppb, can be used differentially to calculate no2_ppb, which may represent a first approximation estimate of the target gas concentration that the electrochemical gas sensor may be exposed to (e.g., expressed in parts per billion (ppb));
(iii) temperature baseline term, modeling non-linear shifts in sensor baseline due to temperature using the following formula (13):

$$T\_baseline = ( ( T - 25 ) \times {c}_{1} + ( {T}^{2} - {25}^{2} ) \times {c}_{2} + {c}_{3} ) \times ( {10}^{6} / 499 ) / escSensitivity , \qquad ( 13 )$$

where this may capture both linear and quadratic effects of temperature on baseline drift.
(iv) relative humidity baseline terms, which may be implemented using exponentially weighted filters with tunable decay constants, may be used to account for time-dependent effects of RH on the sensor baseline, where these features may be computed using a recursive formulation of the form of the following formula (14):

$${RH\_basline}_{t} = Δ RH \times deltaRhConst + {RH\_basline}_{t - 1} \times exp ( - Δ Time / τ ) , \qquad ( 14 )$$

where RH_baselineₜ may be the updated baseline estimate at the current time step, RH_baselineₜ₋₁ may be the previous baseline value, ΔRH may be the change in relative humidity between the current and previous time steps, ΔTime may be the time interval between successive time steps (which may coincide with measurements), deltaRhConst may be a scaling coefficient that adjusts the impact of RH changes, and τ (tau) may be a time constant that governs the rate of exponential decay, and this formulation may allow the baseline to respond dynamically to changes in humidity, while attenuating the influence of past changes over time, where, for example, two RH_baseline features may be computed in parallel, using different values of τ, such as: (a) rh_baseline_1_5 with T=1.5 hours, capturing fast-changing, transient humidity effects, and (b) rh_baseline_8_0 with τ=8 hours, capturing slower, more gradual humidity trends, where, together, these features may enable the calibration model to distinguish between short-term spikes in humidity that may briefly perturb the sensor output and longer-term humidity patterns that may shift the baseline more gradually;
(v) rolling window features, which may be calculated over any suitable period (e.g., a sliding 14-day window) to capture the distribution of recent operating conditions, such as (a) Averages: rh_rolling, temp_rolling, dew_rolling, no2_rolling and (b) Percentiles: no2_rolling_low (20th percentile), no2_rolling_high (80th percentile), where these features may help normalize sensor behavior over time and capture calibration drift;
(vi) time-dependent features, such as elapsed days: days since deployment, which may be used to model long-term sensor drift; and/or
(vii) interaction terms, such as vGas×RH, vGas×T, and/or T×RH, where such terms may allow the model to capture non-linear sensor behavior caused by co-varying environmental parameters.
   The training dataset may be filtered in any suitable way(s), including, but not limited to, to include only sensors with sufficient operational time (e.g., minimum 10 days of collocation), to include only sensors with exposure to a high enough pollutant concentration range (e.g., at least one day with average gas concentrations exceeding 15 ppb for NO₂), and/or to include only sensors passing quality assurance criteria such as high signal stability and absence of extreme baseline drift. Reference data may come from any suitable reference monitors, such as regulatory-grade analyzers that may be synchronized to the same averaging intervals as the sensors. The model trained may be, for example, LightGBM. LightGBM may be selected for its ability to handle high-dimensional feature sets, non-linear relationships, missing values, and/or interpretability through feature importance analysis. Any suitable machine learning regression ensemble may be used to replace LightGBM, including, but not limited to, the following:
(1) Random Forest Regression (e.g., simpler bagged trees rather than boosted, often easier to tune at small data scales);
(2) XGBoost or CatBoost: (e.g., other gradient-boosting libraries with slightly different split algorithms or categorical-handling capabilities);
(3) Support Vector Regression (“SVR”) (e.g., using a kernel function, may be good for non-linear relationships but may sometimes scale poorly on exceptionally large datasets);
(4) Neural Networks (e.g., feedforward MLP) (e.g., may learn arbitrarily complex functions given enough data, though they may require more hyperparameter tuning);
(5) Multiple linear regression (“MLR”) (e.g., a simpler linear model, can leverage derived features to model non-linearities); and/or
(6) Piecewise multiple linear regression (e.g., can help model different pollutant concentration regimes).
   The system may be configured to train a regression model on large collocation datasets to map raw sensor outputs and derived features to reference concentrations. LightGBM is one concrete instantiation but any regression approach that may capture non-linearities without overfitting may be acceptable. On holdout datasets (e.g., from cities excluded during training), the model may consistently improve accuracy over raw NO₂ signals. For example, average R² may increase from ˜0.34 (raw) to ˜0.64 (calibrated), and RMSE and MAE may be reduced by ˜50%. The model may perform best when raw NO₂ signals are within the sensor's nominal operating range (e.g., <200 ppb), and/or when temperature and RH fall within the training domain (e.g., 0-35° C. and 30-95% RH). Although some embodiments may focus on NO₂ gas sensors, the same structure may apply to any sensor type (e.g., so long as the system may replace the pollutant-specific inputs and derived features (e.g., for PM sensors, raw inputs may become count or mass bins, and derived features may include dust ratios, humidity adjustments, etc.; for BC aethalometers, raw inputs may become multiple wavelength attenuation channels, and derived features may include temperature-rate corrections; etc.)). Therefore, the disclosure may outline a template for global calibration, which may include choosing raw inputs relevant to a sensor, compute derived features that may capture environmental and sensor-specific effects, and then train a suitable regression model. The NO₂ example is simply a specific instantiation. While the global calibration model may perform well under typical conditions, limitations may arise in scenarios involving extreme pollution levels or when raw sensor signals are highly biased. Such bias may result from factors including sensor-to-sensor variability, long-term sensor aging, and/or cross-sensitivity to interfering gases. In these cases, model accuracy may be further improved through additional correction strategies, including, but not limited to, the following:
(i) factory characterization to quantify and correct for sensor-specific baseline offsets or sensitivities prior to deployment (e.g., such a process may involve exposing sensors to zero-air and controlled gas concentrations under varied environmental conditions; this may be done in the factory, and may be used to tune the sensor raw output before it is fed to the global calibration; such a decision may depend on cost vs. benefit for specific sensor types (e.g., it might be a good idea to do this for sensor types that exhibit a high device-to-device variation, which global calibrations may struggle to correct for));
(ii) sensor normalization within a group, where multiple sensors may be collocated (e.g., even without a reference monitor) and each sensor's output may be normalized to that of a representative sensor or to the group mean (e.g., a linear correction of the form corrected_output=raw_output×a+b may be applied, where a and b may be determined from group comparisons), where normalization (e.g., aligning each sensor's raw output to a group mean or representative unit) may not be needed for sensors whose data feed into the global-calibration training set (e.g., this may be an option operation the system can apply to any newly produced sensor to “tune” its raw output before or even after the global calibration model may be applied (see, e.g., process 1600 of FIG. 16, process 1700 of FIG. 17, operations 1904 and 1906 of process 1900 of FIG. 19, operations 2006 and 2008 of process 2000 of FIG. 20, etc.)); and/or
(iii) dynamic calibration via periodic collocation, where one or more sensors in the network may be collocated with a reference monitor over time and where the calibration updates derived from this collocation may then be propagated across the sensor network (e.g., dynamic calibration may not be needed for sensors whose data feed into the global-calibration training set (e.g., it may be an optional operation where the one or more sensors collocated with a reference monitor may be used to calculate an additional correction on a rolling window of collocation data, which may then be applied on top of the output of global calibration for sensors deployed in that region (see, e.g., process 2200 of FIG. 22, process 23 of FIG. 23, etc.
   Such global calibration embodiments may support scalable, out-of-the-box deployment of sensors with substantially improved data quality, even in the absence of local reference instruments. In some embodiments, the calibrated output of the global model may be further refined through project-specific collocation-based adjustments or hybrid approaches that combine global and local calibration layers to optimize performance across diverse deployment environments. It will be readily understood by those skilled in the art that similar global calibration models may be constructed for other gaseous pollutants measured by gas sensors, including but not limited to nitric oxide (NO), carbon monoxide (CO), ozone (O₃), hydrogen sulfide (H₂S), sulfur dioxide (SO₂), and ammonia (NH₃), by adapting the model inputs, derived features, and/or training datasets accordingly. More broadly, beyond any specific features and model architectures described herein, a core of this disclosure may lie in a generalizable process of developing global calibrations. This process (see, e.g., process 1500 of FIG. 15) may include (1) collecting large-scale collocation datasets from air quality sensors deployed alongside reference instruments across diverse environmental conditions, (2) computing a set of derived features that capture environmental influences, sensor behaviors, and interactions among variables, and (3) training a machine learning model (e.g., LightGBM or any other suitable regression or ensemble technique) to produce calibrated outputs that are significantly more accurate than the raw sensor measurements. This methodology can be adapted to different sensor types, pollutants, and/or deployment use cases, and may represent a flexible, scalable solution to improving the data quality of low-cost air quality monitoring networks. Therefore, pre-calibration using global calibrations may enable a brand new sensor node to be deployed without a fresh collocation, because it already carries a global calibration derived from historical collocations of other similar sensors (e.g., when the global model was trained beforehand on many prior collocation datasets, such that a newly onboarded sensor may no longer need to be collocated locally (e.g., it can be “calibrated out-of-the-box” using the existing global model)). In practice, such operations (1)-(3) (e.g., collect collocation data, compute derived features, train a model) may be repeated separately for each sensor-pollutant combination. For example, the system may be configured to carry out one workflow for a PM sensor to PM_{2.5} global model, a different workflow for an NO₂ gas sensor to NO₂ global model, a different workflow for a CO gas sensor to CO global model, and yet another workflow for an Aethalometer to BC global model.

In some embodiments, a multivariate linear regression model can be used to account for multiple influencing factors simultaneously, such as ambient temperature, humidity, and cross-pollutant effects. A regression model may be designed to adjust the sensor outputs based on a linear combination of these environmental variables, providing a robust calibration that compensates for potential sensor biases or sensitivities to non-target pollutants. In some embodiments, a stepwise multivariate linear regression may be used to capture how the sensor response to environmental conditions and cross-sensitive pollutants change as the pollutant concentration to which the sensor is exposed changes. In some embodiments, different statistical models may be used to utilize a large amount of collocated measurements to train a calibration model that can be used to enhance the accuracy of measurements from sensors when deployed in diverse environments. In some embodiments, particulate matter (PM_{2.5}) mass concentration measurements from PM sensors may be calibrated using a statistical or machine learning model trained on large-scale collocation datasets. These models may compensate for sensor limitations, such as environmental sensitivity, assumed particle properties, and non-linearity in optical response, by learning from reference-grade instruments across diverse environmental conditions and without requiring for all sensors to be individually characterized. For example, some embodiments may implement a multiple linear regression (“MLR”) model that may be trained on a vast amount of collocation data from any suitable number (e.g., hundreds, thousands, etc.) of sensors across any suitable number (e.g., dozens) of reference sites in varied locations. The model may use both raw and derived features as inputs, including, but not limited to the following:
(i) relHumidity_raw (e.g., internal relative humidity measured by the sensor, which may be used to account for the hygroscopic growth of particles that can alter the scattering properties sensed by optical instruments);
(ii) pm2_5MassConc_raw (e.g., raw PM2.5 mass concentration, which may represent the primary uncorrected output of the sensor);
(iii) pm10MassConc_raw and pm1MassConc_raw (e.g., raw PM10 mass concentration and PM1 number concentration, which may provide size-resolved information on the ambient aerosol and can help capture shifts in particle distribution that may affect the sensor's inference of PM2.5);
(iv) pm_rh interaction: defined as pm2_5MassConc_raw×relHumidity_raw (e.g., an interaction term between PM2.5 and relative humidity, which may reflect the compounding effect of elevated humidity on the particle signal, which may be particularly important since light-scattering sensors tend to overestimate PM2.5 in humid environments); and/or
(v) temperature_minus_dew (e.g., the difference between ambient temperature and dew point, where dew point may be estimated from measured temperature and relative humidity using an empirical formula (e.g., Magnus), where this feature may reflect conditions like fog that may interfere with optical sensing).
   In some embodiments, the model may take the form of the following formula (15):

$$PM 2.5 \_calibrated = {c}_{1} \cdot relHumidity\_raw + {c}_{2} \cdot pm 2 \_ 5 MassConc\_raw + {c}_{3} \cdot pm 10 MassConc\_raw + {c}_{4} \cdot pm 1 NumConc\_raw + {c}_{5} \cdot pm\_rh \_interaction + {c}_{6} \cdot temperature\_minus \_dew + intercept , \qquad ( 15 )$$

where coefficients c₁ through c₆ may be trained on collocation data minimizing the difference between the calibrated sensor output and the corresponding reference measurement (e.g., trained coefficients may be learned from loads of global calibration training (e.g., collocation) data, and the inputs may change dynamically as each individual sensor takes measurements, where the sensor that measures does not necessarily have to have contributed data to the global calibration training data). In some other embodiments, a blended calibration approach may be employed, which may blend two distinct linear regression models to improve accuracy across the full range of observed PM2.5 concentrations. This structure may be particularly effective in scenarios with episodic extreme pollution events, such as wildfire smoke or dust storms. Such a blended model may include or otherwise be composed of any suitable regimes, such as the following:
(i) one model that may be optimized for low to moderate PM2.5 concentrations, generally below 100 micrograms per cubic meter, where this model may use a full feature set that may include humidity, size-resolved particle metrics, interaction terms, and/or the fog proxy feature, and where this model may be designed to correct for typical environmental interferences and nonlinearities that may be present at lower concentrations and may take the form of the following formula (16):

$$M 2.5 \_calibrated = {c}_{1} \cdot relHumidity\_raw + {c}_{2} \cdot pm 2 \_ 5 MassConc\_raw + {c}_{3} \cdot pm 10 MassConc\_raw + {c}_{4} \cdot pm 1 NumConc\_raw + {c}_{5} \cdot pm\_rh \_interaction + {c}_{6} \cdot temperature\_minus \_dew + intercept ; and \qquad ( 16 )$$

(ii) another model that may be optimized for high-concentration conditions, generally above 300 micrograms per cubic meter, where this high-end model may take the form of a simplified linear scaling of the raw PM2.5 signal, recognizing that under extreme conditions, sensors often show saturation or compression effects, where the simpler model may improve robustness by compensating for systematic underestimation or overestimation, and may take the form of the following formula (17):

$$PM 2.5 \_upper = {c}_{7} \times pm 2 \_ 5 MassConc\_raw . \qquad ( 17 )$$

For concentrations between 100 and 300 micrograms per cubic meter, the final calibrated value may be computed as a smooth weighted average of the two models. The weight may vary linearly depending on the raw PM2.5 concentration, ensuring a continuous transition without introducing abrupt changes or discontinuities in the output. This may take the form of the following formula (18):

$$PM 2.5 \_calibrated = ω \times PM 2.5 \_lower + ( 1 - ω ) \times PM 2.5 \_upper , \qquad ( 18 )$$

where ω=(300−pm2_5MassConc_raw)/(300−100), thereby ensuring a smooth transition between regimes. This blended structure may allow the model to preserve interpretability and generalization at lower concentrations, while improving accuracy during high-pollution events by mitigating saturation and scaling errors typical of optical sensors. Empirical testing shows that these calibrations can significantly improve performance. Median R² values may increase by 7-20% and RMSE may decrease by 10-50% relative to raw sensor outputs. The calibrated data may also improve categorical accuracy for air quality thresholds, such as those used in NowCast AQI classifications, especially during wildfire smoke or high-dust events. In some embodiments, these PM_{2.5} global calibrations may be further enhanced through dynamic updates, regional retraining, and/or collocation-based fine-tuning, and/or by the addition of additional features such as dust-sensitive features. It will be readily understood by those skilled in the art that the model framework may also be extended to calibrate PM₁₀, PM₁, total suspended particulate (“TSP”), or other particulate indicators measured by optical sensors by adapting the inputs and regression logic.

In some embodiments, two or more global calibrations may be combined in a hybrid approach that maximizes the benefits of each individual model. In this method, a machine-learning LightGBM model may be used to correct periods of time when the sensor signal is heavily impacted by environmental interferences while a linear regression model may be used when the environmental interferences are limited. At each timestep the final calibrated sensor measurement may be a weighted average of the two individual global model calibrations. A third unsupervised machine learning model may be used to determine the weight at each time step based on environmental readings. This approach may be advantageous as the LightGBM model may be able to correct non-linear responses within the range of data used to develop the model and the linear regression model may be able to extrapolate outside of the range of data used in training. This may enable the approach to maximize the benefits of both models to enhance sensor accuracy. In some embodiments, an unsupervised clustering algorithm, such as KMeans, may be applied to distinguish between periods with high and low environmental interference in the raw sensor signal. The algorithm may use any suitable input features, such as temperature, relative humidity, and the time-dependent RH baseline term. When configured to identify two clusters, the model may effectively separate periods where the sensor is producing relatively stable and accurate readings (e.g., during cool, dry conditions) from those where significant interferences are present (e.g., during rapid RH fluctuations or baseline shifts). This classification can then be used to assign different weights to the outputs of the global calibration models, such as by favoring the LightGBM model during periods of interference and the linear regression model during stable periods. There may be a third model (e.g., a KMeans model) that may be configured to works on outputs of the LightGBM model and on outputs of the linear regression model as well as on any additional inputs (e.g., temperature, relative humidity, and the time-dependent RH baseline term of the sensor being calibrated) to provide the final output. For example, in some embodiments, a LightGBM ensemble may be configured to correct the raw output (e.g., in “high-interference” conditions), a linear regression may be configured to correct the raw output (e.g., in “low-interference” conditions), and a KMeans clustering, or similar unsupervised model, may be configured to use environmental inputs (e.g., T, RH, RH_baseline, etc.) to assign each timestamp to a “low-interference” or “high-interference” cluster. At each time step, the clustering result may be configured to determine the weight given to the LightGBM output versus the linear regression output. In other words, the KMeans model may be configured to take as input environmental features (e.g., T, RH, RH_baseline, etc.) and/or the raw or partially calibrated output, and to produce a “cluster label” (e.g., low vs. high interference), which may then be used to blend the two calibrated values into a final, adaptive output. Thus, the KMeans may be effectively the third model that may be configured to decide how to weight the other two. This may enable adaptive calibration based on real-time environmental conditions. In one study, approximately 65% of measurements were classified as low-interference and 35% as high-interference, with significantly better accuracy observed in the former cluster. This clustering-based weighting scheme may improve model robustness by dynamically adapting to known sources of signal distortion in electrochemical gas sensors.

The global calibrations may be able to correct baseline sensor outputs, adaptively respond to environmental changes, and/or remove sensor bias, offset, drift, cross-sensitivity to non-target pollutants or environmental factors, ensuring high accuracy of calibrated measurements across different geographies and conditions.

### Collocation-Based Calibration

Collocation-based calibration may begin by placing one or more air quality sensors in close proximity to a high-accuracy reference monitor (see, e.g., process 1400 of FIG. 14, operation 1808 of process 1800 of FIG. 18, etc.). This positioning may ensure that both the sensor being calibrated and the reference monitor are exposed to identical or substantially identical atmospheric conditions, thereby capturing the same air mass along with consistent pollutant concentrations and environmental factors. This setup may be critical for establishing a reliable baseline from which accurate calibrations can be derived.

To ensure the calibration remains valid across typical operational conditions that the sensors will face post-deployment, the collocation may be conducted during a time period (e.g., a month) where the variability in pollutant concentrations and environmental conditions may be representative of long-term variability. This time period may be selected based on historical climate data to include a broad spectrum of environmental conditions (e.g., ranging from temperature and humidity to pollutant concentrations) that may be characteristic of the target deployment area. The goal may be to capture the variability that sensors are likely to encounter, ensuring the calibration process accounts for real-world environmental dynamics. In some embodiments, the representative time period may be selected to maximize day-to-day variability in pollutant concentrations while also ensuring that ambient temperature and relative humidity distributions fall within the expected operational range. For example, a representative time period that includes both low and elevated pollutant concentrations, spans the 25th to 75th percentiles of historical RH and temperature distributions, and exhibits no abnormal reference instrument behavior may be selected. In another embodiment, a calibration transfer strategy may be implemented in which one or more sensors may remain permanently collocated with a reference monitor for an extended period (e.g., one year) in order to experience a broad range of environmental conditions typical to the region, while other sensors may be collocated for shorter durations (e.g., one month). These short-term sensors and the long-term sensor may be normalized based on their overlapping data. The long-term sensor's calibration profile (e.g., developed using its full dataset) can then be applied to the normalized output of the short-term sensors. This method may enable improved accuracy for a broader fleet of sensors without requiring each one to undergo a year-long collocation, improving representativeness of calibration while reducing operational effort. Short-term and long-term sensors do not strictly have to be from the same factory batch, but they ought to be the same model type so that their raw outputs may be comparable. In some embodiments, a same sensor model may be used, such as where both sets should use identical hardware (e.g., same sensor head, same electronics revision, etc.), so that differences in readings may reflect environmental drift rather than hardware mismatches. In some embodiments, linear scaling for alignment may be used, such as where no global model may be used (e.g., simple linear scaling or normalization of the two sensors raw output).

The duration of collocation may be set at a minimum of any suitable amount of time, such as one month, that may be able to provide enough time to accumulate comprehensive environmental data under varied conditions. Throughout this period, continuous monitoring may be conducted, not only of pollutant levels but also of critical atmospheric parameters such as temperature, humidity, and other relevant conditions that might influence sensor readings. This extensive monitoring may be crucial for gathering the necessary data to support effective calibration. It is important to note that the calibration developed during this period may be specifically tailored to the environmental and pollutant conditions experienced during the collocation. If the sensors are later deployed in conditions significantly different from those recorded, the calibration may not provide accurate corrections, highlighting the importance of selecting an appropriate representative month for collocation.

In developing collocation-based calibrations, both traditional statistical methods and advanced machine learning models may be trained on collocation data. In some embodiments, data from the sensor being calibrated may be augmented by integration with additional environmental or pollution sensors data, for example incorporating, in some embodiments, simultaneous readings of temperature (T) and relative humidity (RH) sensors exposed to the same air sample as the sensor being calibrated to accurately measure and correct for environmental influences. In some embodiments, the simultaneous readings of T, RH, and/or any other suitable parameters ought to come from sensors that go together with the sensor being calibrated (e.g., in the same enclosure, electrically coupled, or just always placed nearby), such that they may keep being available when the sensor is moved to the field. In some embodiments, derived features may be calculated together with readings from the sensor being calibrated and readings from additional environmental sensors exposed to the same sample of air. These derived features may include time dependent or interaction terms to capture non-linearities in sensors' response to the environment. All sensors may be a part of the same sensor node or sensor device that may operate multiple sensors (e.g., a PM sensor, an NO₂ sensor, a T sensor, an RH sensor, etc.). These sensors may provide data crucial for developing complex features, such as time-dependent relative humidity baseline adjustments. These advanced metrics and features may be integral to refining the calibration model to accurately reflect the dynamic nature of environmental influences on sensor performance.

Once the collocation-based calibrations have been developed, they may undergo a rigorous process of refinement and validation. This may include the use of statistical tests and validation datasets to ensure the accuracy and robustness of the model(s), confirming their ability to reliably predict true environmental conditions and adjust sensor outputs accordingly. In some embodiments, techniques, such as cross-validation, may be employed to ensure that the model generalizes well across different geographical locations and environmental settings. In some embodiments, the calibration models may be trained using procedures specifically designed to ensure robust generalization and avoid overfitting to the collocation dataset. Within a training set, a rigorous cross-validation framework may be employed. In one embodiment, k-fold cross-validation may be repeated multiple times, such as, for example, 10-fold cross-validation repeated 5 times, to test different combinations of input features, regularization strategies, and/or preprocessing techniques. Candidate models may be evaluated based on any suitable metrics, such as R², RMSE, and/or MAE, and the feature set that consistently delivers the best generalization performance across folds may be selected. Multiple collocation models may be built simultaneously on the same data for the same particular collocated sensor and tested in parallel to determine the best one for future use in the field. For example, multiple collocation-based calibration models may be temporarily built and compared in terms of performance metrics to select the best one at the end. To further enhance model reliability, quality filters may be applied during training to remove data points that are likely to introduce bias or noise. These may include measurements taken during known sensor warm-up periods, periods with high RH variability, and/or times when pollutant concentrations fall below detection thresholds. In some embodiments, parameter sweeps over time constants (e.g., for baseline correction terms) may be incorporated into the model selection loop to identify optimal decay values for transient environmental effects. Once the best model structure is identified through cross-validation, the final calibration model may be fit on the entire training dataset and validated on the held-out test set to estimate real-world performance.

Upon validation, these calibrations may be applied to the sensors, systematically adjusting their raw data outputs to align closely with the data recorded by the reference monitor. This application can be executed in real-time through embedded software within the sensor or cloud software enhancing the data streamed by the sensors in real-time, or retrospectively through post-processing of collected data.

After calibration, performance metrics, such as R² (coefficient of determination) and RMSE (root mean square error), may be calculated. These metrics may quantify the improvement and reliability of the sensor data post-calibration, providing a clear measure of how well the calibrated data matches the reference data.

To ensure the long-term accuracy of the sensors, continuous monitoring and periodic recalibration may be recommended, especially if the deployment environment undergoes significant changes that were not accounted for in the initial calibration. This long-term monitoring may be vital for maintaining the calibration's accuracy and reliability over time.

### Layered Calibration

The layered calibration approach may provide a comprehensive method for calibrating air quality sensors, integrating global calibration with collocation-based calibration to enhance measurement accuracy and reliability (see, e.g., process 2100 of FIG. 21, process 2200 of FIG. 22, process 2300 of FIG. 23, etc.). This layered calibration approach may address both broad and specific environmental and temporal variations, ensuring that sensor outputs may be finely tuned for diverse deployment conditions.

The layered calibration strategy may begin with a global calibration process, which may standardize the baseline outputs of the air quality sensors. This initial operation or set of operations may involve collecting extensive environmental data from sensors collocated with reference monitors across multiple locations and conditions. Using this data, advanced statistical methods and machine learning techniques may be employed to develop a global calibration model that adjusts sensor outputs to improve alignment with reference grade monitors for common biases (e.g., deviation from reference monitor measurements) and/or environmental influences. This global calibration may be applied to each sensor before deployment, ensuring uniformity and a consistent level of accuracy across all sensors, regardless of their specific deployment locations.

Following the global calibration, a more detailed collocation-based calibration may be conducted. Sensors may be placed next to high-accuracy reference monitors for a specified period, typically one month, during which both the sensors and the reference monitors record data under identical environmental conditions. The tailored calibration may then be applied to the sensor outputs after applying global calibration to those outputs, fine-tuning them for higher accuracy in the specific deployment environment. In some embodiments, after applying a global calibration model to NO₂ sensor outputs, a collocation-based scaling operation may be used to further reduce residual bias. In this approach, the globally calibrated sensor values may be linearly regressed against the collocated reference instrument over a short overlapping period, such as 30 days. The result may be a simple linear transformation that adjusts the calibrated signal to better match the reference scale. This may work as a second collocation calibration model running on the outputs of a global calibration model running on the inputs of a particular sensor. For example, after applying a global calibration to a sensor's raw data (e.g., producing a “globally calibrated” measurement), the system may be configured to develop a second calibration on those already-corrected values. This may provide a two stage process, where, at stage 1, sensor raw has global model applied thereto for generating global-calibrated output, and, then, at stage 2, those global-calibrated outputs may be used to fit a simple linear regression (or other local model) to the reference over the collocation period, where the resulting correction (e.g., slope and intercept) may become the second calibration to be used on the field. This two-step process (e.g., global to collocation-based) may ensure that major environmental interferences are already removed, so the collocation regression only needs to correct residual scale or offset errors. As this method may assume that the global model has already accounted for major environmental interferences, such as temperature and humidity, there may be no need to ensure the collocation period is representative of seasonal variability. In some embodiments, the method may include constraints to ensure the 30-day period spans a sufficient range of NO₂ concentrations to avoid overfitting. When tested across long-term deployments, this approach has been shown to reduce the mean bias error (MBE) and improve RMSE and MAE, without affecting the R² of the global model, which may remain fixed due to the univariate nature of the scaling regression. This may provide a practical option for projects that can accommodate limited collocation, offering an intermediate between purely global and fully local calibration strategies. In some embodiments, this period of collocation may be chosen to be representative of the typical environmental conditions at the deployment site, capturing a wide range of variations in temperature, humidity, and pollutant concentrations. The calibration developed during this period may be specifically tailored to these conditions, making it crucial to select an appropriate representative month.

In some embodiments, not all sensors may undergo direct collocation-based calibration. Instead, the fine-tuning achieved through collocation can be algorithmically transferred to other sensors, leveraging the standardized data provided by the global calibration. This transfer of calibration may ensure that all sensors in the network may benefit from the localized adjustments, even if they were not individually collocated.

In some embodiments, a group of sensors may be normalized before applying global calibration. In some embodiments, sensors may be installed side by side in a controlled environment, such as a parking lot, for a period of two weeks to a month. During this period, normalization factors may be calculated to align each sensor's output to the mean output of the group or to the output of a select sensor within the group (e.g., this process may be done at the factory or by the end user or by some in between service provider). This process may involve fitting a simple linear transformation to each sensor's raw signal, such as using a slope and intercept, that may reduce sensor-to-sensor variation within the group. In some embodiments, normalization may be performed at the factory, either in a chamber or through outdoor exposure to a common air mass. Studies have shown that sensor-to-sensor correlation for NO₂ typically may remain stable over time scales of up to two years. For example, correlation matrices of raw NO₂ readings across 12 sensors deployed continuously at the same site in Sacramento revealed R² values consistently above 0.9 across seasons and years for most sensors. This indicates that normalization relationships derived at one time are likely to hold over lengthy periods, allowing for effective calibration transfer even months or years after initial characterization. Additionally, linear scaling parameters used for normalization, such as slope and intercept, have been found to vary only modestly across seasons. In a monthly evaluation of one sensor scaled to a fixed peer, the variation in the adjusted output for a 20 ppb NO₂ input signal was typically within 2-4 ppb. Only a few sensors showed larger variability (e.g., up to 7-10 ppb), often due to specific device-level or environmental responses. Furthermore, sensors that were collocated in previous years and later re-collocated after deployment continued to show high inter-sensor correlation with their peers, supporting the robustness of this normalization approach. These findings suggest that normalization factors applied to groups of sensors, especially when drawn from similar manufacturing batches or deployed in similar environments, can remain valid over multi-year periods, significantly reducing the need for repeated colocations. After normalization, the global calibration model may be applied to the corrected signals, improving measurement consistency and ensuring that inter-sensor differences do not distort air quality assessments or downstream analyses. In some embodiments, normalization may be carried out at the factory, either indoors in an environmental chamber or outdoors. This method may mitigate device-to-device variations arising from manufacturing discrepancies. After normalization, the global calibration model may be applied to the normalized outputs, thereby enhancing uniformity and reliability across the sensor network. Such normalization can be done in any suitable situation, such as on raw data before feeding it to the global calibration, on the globally calibrated data before publishing it, or before feeding it to the subsequent collocation-based calibration.

Further refining the calibration process, some embodiments may include dynamic calibration. In this approach, one or more sensors may be continuously or periodically collocated with reference monitors, and the collocation data may be regularly reviewed (e.g., daily, weekly, or monthly) to update a collocation-based calibration. These updates may then be applied to all sensors in the field, optimizing their performance according to local and seasonal variations. Dynamic calibration may ensure ongoing accuracy by continuously adjusting the sensors to reflect the latest environmental data.

Various embodiments may combine these calibration strategies to suit specific project requirements. For example, any combination of normalization, global calibration, and collocation-based calibration (e.g., dynamic collocation-based calibration) may be employed. This approach may start with normalizing sensor outputs to a common baseline, applying a global calibration for enhanced accuracy, and then incorporating dynamic updates from ongoing collocation studies. By integrating global standardization with localized fine-tuning and dynamic adjustments, the layered calibration approach may ensure optimal sensor performance across a wide range of conditions.

### Calibration of Black Carbon Monitors Based on Temperature Change Rate

Black carbon monitors that rely on optical attenuation through a filter medium can experience baseline signal shifts under changing environmental conditions, even when sampling particle-free air. These baseline fluctuations may be particularly pronounced during periods of rapid ambient temperature change, which can affect the optical and electronic components of the device or alter internal airflow dynamics. Without correction, these shifts may appear as false changes in black carbon concentration, thereby degrading data quality and potentially leading to misinterpretation of air pollution levels. Baseline correction may therefore be essential in some embodiments to ensure reliable and accurate BC measurements in real-world, outdoor environments, where temperature changes frequently. In some embodiments, the calibration may begin with characterizing each black carbon monitor during production. This process may involve placing the monitor in an environment where it samples clean air, ensuring that no particulate matter interferes with the calibration. For example, a HEPA filter may be fitted to the monitor's air sample inlet. The monitors may then be subjected to controlled temperature ramps that, in one embodiment, exceed 2 degrees Celsius per hour. These ramps may be tested through two full cycles, including two peaks and one trough, to comprehensively capture the monitor's response to temperature variations.

During this process, the outputs of the black carbon monitor at various light frequencies may be recorded along with the internal temperature change rate of the unit. In some embodiments, a linear regression may be calculated between these outputs and the temperature change rates. To minimize the influence of outliers, Theil-Sen regression slopes may be used for determining the regression coefficients. In some embodiments, the unit may pass the characterization if the slope of the regression is less than 120 degrees Celsius per hour.

Following the characterization, the temperature rate of change calibrations may be applied to the monitor's output. In some embodiments, the coefficients obtained from the linear regressions during the factory characterization may be used to correct the outputs at various light frequencies of the black carbon monitor. This correction can be implemented either directly within the device firmware or through cloud-based software.

Next, using the calibrated outputs, derived measurements, such as smoothed black carbon concentration outputs at different light frequencies and source apportionment estimates, may be recalculated. This may ensure that all reported values reflect accurate concentrations unaffected by temperature change rates, providing reliable and consistent data for environmental monitoring.

By implementing this calibration method, the accuracy and reliability of black carbon monitors may be significantly enhanced, making them more effective tools for environmental health assessments and air quality studies, especially when deployed outdoors in a stationary setting.

In some embodiments, field characterization of one or more black carbon monitors may be used to calculate a generalized relationship between rate of change of temperature and baseline shift. When a black carbon monitor is deployed to the field, it may measure both black carbon concentration and temperature rate of change. The generalized relationship may be used to apply a generalized correction to the black carbon concentration outputs. Some embodiments may include calculating a device-specific linear correction factor between the internal sample temperature rate of change and the sensor's baseline output, especially for light attenuation channels such as the infrared (“IR”) and blue channels. During production, each unit may be exposed to a controlled temperature profile (e.g., in a testing chamber) while sampling clean air (e.g., via HEPA filtration), and the resulting sensor output may be used to determine the slope and offset of the linear relationship between sensor baseline and temperature. These slopes may then be stored in each device's firmware or on a cloud platform and applied during operation by the sensor or cloud server to subtract estimated baseline bias in real time. In some embodiments, a standardized temperature correction model may be derived from a representative group of sensor units. This model may include average correction coefficients for the IR and blue channels, designed to compensate for the baseline shift across devices of the same type. This approach may allow for correction when individualized chamber testing may not be feasible and may ensure consistent performance across large deployments. In some embodiments, a time-lag compensation mechanism may be incorporated. As the internal sample temperature may lag behind the external housing temperature due to thermal insulation, the rate of change used in the correction algorithm may be calculated using a smoothed or delayed temperature signal. This may reduce overshoot or under-correction of baseline drift, especially during rapid ambient changes. In some embodiments, environmental shielding strategies, such as thermal insulation or sunshades, may be used to reduce the magnitude of temperature change experienced by the sensor. These correction embodiments may enable the black carbon monitor to maintain stable baseline output even during deployments with significant diurnal or weather-driven temperature variations, thereby supporting continuous and accurate long-term outdoor air quality measurements.

### Additional Systems and Methods

A system with a sensor node containing various environmental sensors to measure air pollutant concentrations can be paired with accessory modules to enhance monitoring capabilities. This design may keep the core functionality cost low and may use accessories as needed, providing flexibility. For instance, the sensor node can perform basic pollutant measurements, while specialized modules can be attached for additional parameters when required.

A communication protocol may be defined to ensure compatibility with third-party instruments, thereby enabling other manufacturers to create devices that may work seamlessly with the sensor node. In some embodiments, the system may be configured to support interoperability with external accessory modules by specifying a communication protocol over RS-485 serial connection, allowing the sensor node to act as a controller and the accessory module as a responder. The protocol may define commands for waking the accessory, initiating and retrieving measurements, reporting firmware versions, and issuing power control signals. A structured message format with delimiters and checksums may ensure robust communication. These modules ought to meet requirements for power consumption, data formatting, and error handling to ensure compatibility. In some embodiments, accessory modules may be expected to respond to sensor node commands, such as wake-up, start measurement, and send measurement within defined time constraints (e.g., within 10 seconds for command readiness and 3,600 seconds for measurement). Measurement results may be formatted using specified numerical constraints (e.g., floats with up to seven significant digits, integers in predefined ranges, etc.) and structured according to a telemetry and sensor data schema. Modules may return fields like mean, max, and min for various sensor channels, alongside a coded error status if applicable. The interface may also support sleeping modes and error recovery to optimize power usage in outdoor deployments. This interface may allow for scalable integration of off-the-shelf or custom instruments into the platform while maintaining data standardization, reliable operation, and centralized control from the sensor node firmware. This may transform the sensor node into a versatile monitoring platform that can be customized and expanded according to specific monitoring needs.

Cloud software may be employed to automatically configure the sensor node and any connected accessory modules. This automation may reduce the need for extensive on-site configuration, making deployment and maintenance more efficient and less error-prone. The cloud-based system can update settings and calibrations remotely, ensuring optimal performance.

In some embodiments, a sensor node may measure particulate matter, while an accessory module may measure black carbon. This setup may allow for collocated measurements, providing comprehensive data on both particulate matter and black carbon concentrations. The sensor node and accessory module may be collocated with one another or it may be two separate independent devices, such as two sensor nodes that may measure different things. In some embodiments, a single integrated device can measure both parameters, thereby simplifying the hardware setup and reducing potential points of failure. In some embodiments, the system may use black carbon measurements to enhance air quality analysis during episodic events, such as wildfires. A black carbon module may provide real-time data on total BC concentration via its infrared channel and further apply source apportionment models to estimate the contributions of different emission sources, such as wood burning and traffic-related combustion. In some embodiments, this source apportionment may be carried out onboard using an aethalometer-style algorithm that may rely on dual-wavelength light absorption measurements (e.g., IR and blue channels). The model may assume different Angstrom Absorption Exponent (“AAE”) values for wood burning and fossil fuel sources and may partition the measured BC accordingly. While default AAE values may be used (e.g., 1.0 for traffic, 2.0 for wood burning, etc.), more refined estimates based on literature (e.g., 0.9 and 1.68) may be applied offline to improve interpretability. During wildfire episodes, the system may observe strong correlations between BC and PM_{2.5} concentrations. In some embodiments, this correlation may be used as a qualitative indicator that elevated PM_{2.5} levels are of combustion origin. For example, when the Pearson correlation between BC and PM_{2.5} exceeds a threshold (e.g., R>0.9), the system may flag the event as likely dominated by combustion emissions, such as smoke from wildfires. In such cases, PM_{2.5} measurements may be interpreted in the context of exposure to fine combustion aerosols, which have heightened health relevance. In some embodiments, the system may also consider periods in which BC and PM_{2.5} show weak or no correlation (e.g., R<0.5) over multi-hour or daily time windows. This condition may indicate that the elevated PM_{2.5} levels are primarily driven by non-combustion sources, such as resuspended dust, sea salt, secondary aerosol formation, or construction activity. By detecting such decoupling between PM_{2.5} and combustion-specific tracers, the system may generate metadata flags indicating likely non-combustion particulate episodes, enabling more accurate air quality interpretation and reporting. In some embodiments, black carbon data may be used to contextualize PM_{2.5} trends in real time. Because BC is a more chemically specific marker for combustion emissions, whereas PM_{2.5} includes both primary and secondary particles from diverse sources, the relative patterns of BC and PM_{2.5} may be used to disaggregate and interpret pollution dynamics. For example, simultaneous increases in both signals may suggest fresh combustion input, while a divergence may indicate atmospheric aging or secondary particle formation. Finally, in some embodiments, the system may combine black carbon readings with meteorological data (e.g., wind direction, temperature rate of change, or vertical mixing conditions) to better understand local air quality dynamics and the movement or source of pollution plumes. This can be particularly useful for identifying episodic intrusions of smoke from distant fires, or distinguishing local versus transported pollution events. These embodiments illustrate how the integration of black carbon sensing with PM_{2.5} measurements and source attribution algorithms may enable more accurate classification of pollution sources, real-time episode detection, and actionable environmental intelligence, particularly during complex, high-impact air quality events, such as wildfire smoke, dust storms, or industrial incidents. In some embodiments, the sensor node may measure particulate matter using a low-cost optical particle sensor, while an accessory module may independently measure particulate matter using a different class of low-cost optical sensor. This second sensor, integrated within the accessory module, may employ dual laser diodes with differing polarization states to enhance particle detection across a broader size range. In particular, the use of polarized laser beams may improve the ability to distinguish particle sizes based on angular light scattering, increasing sizing accuracy in accordance with Mie scattering theory. This configuration may enable the accessory module to more effectively characterize larger particles (e.g., with aerodynamic diameters greater than 1 micrometer), thereby enhancing the accuracy of coarse particulate matter measurements, such as PM₁₀ and total suspended particulate (“TSP”). When combined with a primary sensor node, the system can provide more complete particle size distribution data and more robust PM measurement performance in environments characterized by atypical or variable particle profiles. In some embodiments, this setup may be particularly advantageous in locations with high levels of dust resuspension or mechanical disturbance, such as mining operations, construction sites, unpaved roadside areas, and industrial perimeters. By utilizing two complementary sensing modalities, the system may better capture both fine and coarse fractions, improving monitoring resolution for occupational health, regulatory compliance, or environmental impact assessment.

Some embodiments may use solar power to operate the sensor node and accessory modules or the combined device. Solar power may enhance the system's sustainability and may make it suitable for deployment in remote or off-grid locations where traditional power sources are unavailable.

Additionally, a method may be provided to utilize black carbon measurements to improve the accuracy of particulate matter measurements. By using source attribution estimates, the method may enhance the conversion of particulate matter sensor data from particle number to mass concentration. This approach may increase the reliability and accuracy of air quality measurements, providing better data for environmental monitoring.

### FIG. 14—Develop Collocation-Based Calibration

FIG. 14 is a flowchart of an illustrative process 1400 for developing collocation-based calibration (e.g., for a particular SOI or a particular SOI pollutant (e.g., at a particular location)). At operations 1402 of process 1400 (e.g., at one or more of operations 1406-1414), collocation for an SOI may be performed at a location (e.g., a location X), while a calibration for at least one SOI (e.g., a collocation-based calibration) may be developed at operations 1404 of process 1400 (e.g., at one or more of operations 1416-1426). For example, process 1400 may start by advancing to operation 1406, where any suitable sensor of interest (“SOI” (e.g., target sensor component (e.g., of a sensor node))) and, optionally, any suitable CSCs (e.g., one or more sensing capabilities configured to remain collocated with the SOI) may be collocated with any suitable reference monitor (“RM”) of the SOI (e.g., a monitor configured to measure the same target pollutant as the SOI (e.g., PM_{2.5})) at any suitable location (e.g., a location X). At operation 1408, any suitable collocation data may be collected from all available sources (e.g., the RM, the SOI, any CSC(s), etc.) for any suitable collocation period of time (e.g., at least 4 weeks). For example, operation 1408 may populate a data table (e.g., each column may be one of an SOI reading, an RM reading, a CSC reading, etc., while each row may be a time stamp for the readings of that row (e.g., as collected at least once an hour for the collocation period (e.g., SOI readings may be captured every 15 minutes, while RM readings may be captured every 60 minutes, whereby every 4 SOI readings may be averaged or otherwise combined into a single reading to be aligned with the same time basis as the RM, etc.). At operation 1410, any suitable collocation data quality checks may be applied and invalid measurements may be filtered out (e.g., any suitable data cleaning may be applied to the data table (e.g., to get rid of any suitable noise, etc.)). At operation 1412, it may be determined whether the collocation data satisfies any relevant requirements (e.g., if the data table includes at least a certain range of high/low reference readings, range of pollutant concentrations, etc.). If no, then process 1400 may return to operation 1408 for at least some suitable additional collocation period of time (e.g., at least one week), otherwise process 1400 may advance to operation 1414, where the collocation data may be frozen (e.g., save, archive, record, or otherwise appropriately handle the collocation data for later use). Next, once collocation has been performed at operation 1402 (e.g., at operations 1406-1414) for an SOI, process 1400 may advance to operation 1404, where a calibration may be developed (e.g., a collocation-based calibration for the SOI (e.g., determine a mathematical translation that may map the detected SOI data and any detected CSC data to the detected RM data). For example, at operation 1416, the frozen collocation data may be augmented by computing any suitable derived features (e.g., mathematical transformations (e.g., derived features that may capture non-linearities, temporal dynamics, and/or event-driven anomalies in the raw data), where, for example, the data table of the frozen colocation data may be augmented by adding any suitable new columns of data (e.g., a product of two CSCs, RH baseline, dust-sensitive squared differences, ratio features, etc.)). At operation 1418, the augmented collocation data may be split into a training data set and a testing data set (e.g., some rows of data may be considered training data sets and some other rows of data may be considered testing data sets (e.g., using any suitable methodology (e.g., random, classic, etc.) At operation 1420, any suitable model (e.g., linear regression, LightGBM, etc.), any suitable features (e.g., any suitable column(s) of the augmented data (e.g., SOI & T, SOI & T & RH, SOI & T*RH, etc.)), and any suitable hyperparameters (e.g., learning rate, number of leafs, depth of tree, and/or the like for LightGBM model, etc.) may be selected (e.g., using any suitable methodology) and then trained using the training data set. At operation 1422, the calibration may be evaluated on the testing data set (e.g., the model selected and trained at operation 1420 may be tested at operation 1422). At operation 1424, it may be determined whether any suitable accuracy targets have been met based on the calibration evaluation. If no, then process 1400 may return to operation 1420 for another loop of operations 1420-1424 (e.g., where another suitable combination of model, features, and hyper parameters may be selected for evaluation), otherwise process 1400 may advance to operation 1426, where the calibration model, features, hyperparameters, coefficients (e.g., weights (e.g., as may be learned from training the calibration)), and/or any other suitable data of the successful evaluation may be recorded or archived or stored or otherwise appropriately defined as a calibration (e.g., as a collocation-based calibration for the SOI). Next, once the calibration has been developed and recorded, process 1400 may advance to operation 1428, where such collocation-based calibration may be ready for use (e.g., in the field with outputs from the SOI (e.g., for use by a calibration regulator 950 of the SOI)).

It is understood that the operations shown in process 1400 of FIG. 14 are only illustrative and that existing operations may be modified or omitted, additional operations may be added, and the order of certain operations may be altered.

### FIG. 15—Develop Global Calibration

FIG. 15 is a flowchart of an illustrative process 1500 for developing global calibration (e.g., for a particular SOI pollutant (e.g., PM_{2.5})). At operation 1502 of process 1500, collocations at any suitable number N locations (e.g., locations 1, 2, . . . , N) with diverse environmental conditions and pollution sources may be performed (e.g., operation 1504 may perform collocation for location 1, operation 1506 may perform collocation for location 2, and operation 1508 may perform collocation for location N, etc.). For example, operation 1504 may be similar to operation 1402 of process 1400 for any suitable SOI at any suitable location 1, operation 1506 may be similar to operation 1402 of process 1400 for any suitable SOI (e.g., the same SOI or a different SOI as the SOI for operation 1504) at any suitable location 2 (e.g., a location different from location 1 of operation 1504), and operation 1508 may be similar to operation 1402 of process 1400 for any suitable SOI (e.g., the same SOI or a different SOI as the SOI for operation 1504 and/or operation 1506) at any suitable location 3 (e.g., a location different from location 1 of operation 1504 and location 2 of operation 1506), where N may be any suitable number and the locations may vary greatly, as may the SOIs (e.g., operation 1502 may involve a large number of operations 1504-1508 utilizing as many SOI collocations as possible throughout the world that may be available to the system (e.g., all previous collocations carried out by all customers in all situations and/or the like (e.g., thousands if not millions)). Operation 1502 may result in a large number of frozen collocation data tables (e.g., as described by operation 1414 of process 1400) for a particular SOI type (e.g., a particular SOI pollutant) (e.g., historical colocation data). At operation 1510, over time, the collocation data from the multiple locations (e.g., from operations 1504-1508 of operation 1502) may be combined into a global collocation dataset (e.g., by stitching together each of the data tables into a megatable that may include a large number of rows spanning all data collection times from all data tables). At operation 1512, exploratory data analysis may be performed on the global collocation data set. At operation 1514, it may be determined whether the analysis reveals sufficient environmental conditions and pollution sources variability (e.g., if the megatable includes data sets collected from various enough locations (e.g., Egypt and Alaska) and/or from various enough time periods to provide a sufficiently global set of data). If no, then process 1500 may return to operation 1502, otherwise process 1500 may advance to operation 1516, where the global collocation data may be frozen (e.g., similarly to operation 1414 but for the global collocation data). Next, at operation 1518, a global calibration may be developed on the global collocation data (e.g., similar to operation 1404 but for the frozen global collocation data of operation 1516 rather than for the frozen collocation data of operation 1414). Then, at operation 1520, the global calibration may be ready for use (e.g., in the field with outputs from a new sensor node (e.g., for use by a calibration regulator 950 of a new SOI)). Therefore, process 1500 may be used by any suitable system of the disclosure to create (e.g., purposely) or utilize otherwise created collocation in diverse environments for at least one sensor type (e.g., PM2.5 sensor as SOI of each collocation) and craft at least one global calibration that may be representative of a wide range of locations and pollutant exposures for the at least one sensor type and then pre-configure certain new sensors (e.g., new sensor nodes) with such global calibration in order to work better out-of-the-box (e.g., if collocation of that sensor may be difficult (e.g., an end user may not have access to an applicable reference monitor)). An SOI that may be configured to apply such global calibration may not be a sensor that was used in developing such global calibration, but instead may be a sensor not yet used in a field but may still be in a factory being readied to provide to an end user.

It is understood that the operations shown in process 1500 of FIG. 15 are only illustrative and that existing operations may be modified or omitted, additional operations may be added, and the order of certain operations may be altered.

### FIG. 16—Develop Raw Normalization

FIG. 16 is a flowchart of an illustrative process 1600 for developing raw normalization. At operation 1602 of process 1600, any suitable number of SOIs (e.g., SOIs for a particular SOI pollutant (e.g., PM_{2.5}) may be installed side by side at any suitable location (e.g., a parking lot or otherwise (e.g., by a manufacturer at a factory or by an end user)). This may be done just for SOIs of a particular type and may not involve any RMs and/or any CSCs. At operation 1604, raw SOI readings may be collected from the SOIs for any suitable collection period of time (e.g., at least 4 weeks), which may be similar to operation 1408 but just for SOI data and not also any RM data and/or any CSC data. At operation 1606, the entire collection period may be divided into any suitable time intervals and, for each time interval, the median raw reading across all SOIs may be calculated (e.g., a group median reading). As there may not be an RM available or used in such a process, the system may be configured to create a virtual RM by using a group median reading, which may be the literal median reading or an average value of all the readings or the reading of a representative median SOI, or the like. At operation 1608, for each SOI, a simple linear regression may be performed between its raw SOI readings and the group median readings to determine slope and intercept. At operation 1610, for each SOI, the slope and intercept may be recorded as the raw normalization for the SOI. Then, at operation 1612, the raw normalizations may be ready for use (see, e.g., process 1900 of FIG. 19).

It is understood that the operations shown in process 1600 of FIG. 16 are only illustrative and that existing operations may be modified or omitted, additional operations may be added, and the order of certain operations may be altered.

### FIG. 17—Develop Global Calibration Normalization

FIG. 17 is a flowchart of an illustrative process 1700 for developing global calibration normalization. At operation 1702 of process 1700, any suitable number of SOIs (e.g., SOIs for a particular SOI pollutant (e.g., PM_{2.5}) may be installed side by side at any suitable location (e.g., a parking lot or otherwise (e.g., by a manufacturer at a factory or by an end user)). This may be done just for SOIs of a particular type and may not involve any RMs and/or any CSCs. At operation 1704, at least one calibration regulator for each SOI may correct its SOI measurements with global calibration (e.g., global calibration that may have been readied for use at operation 1520 of process 1500). At operation 1706, global calibrated SOI measurements may be collected from the SOIs for any suitable collection period of time (e.g., at least 4 weeks), which may be similar to operation 1408 but just for SOI data and not also any RM data and/or any CSC data. At operation 1708, the entire collection period may be divided into any suitable time intervals and, for each time interval, the median global calibrated measurement across all SOIs may be calculated (e.g., a group median measurement). As there may not be an RM available or used in such a process, the system may be configured to create a virtual RM by using a group median measurement, which may be the literal median global calibrated measurement or an average value of all the global calibrated measurements or the global calibrated measurements of a representative median SOI, or the like. At operation 1710, for each SOI, a simple linear regression may be performed between its global calibrated SOI measurements and the group median measurements to determine slope and intercept. At operation 1712, for each SOI, the slope and intercept may be recorded as the global calibration normalization for the SOI. Then, at operation 1714, the global calibration normalizations may be ready for use (see, e.g., process 2000 of FIG. 20).

It is understood that the operations shown in process 1700 of FIG. 17 are only illustrative and that existing operations may be modified or omitted, additional operations may be added, and the order of certain operations may be altered.

### FIG. 18—Use Collocation-Based or Global Calibration

FIG. 18 is a flowchart of an illustrative process 1800 for using calibration. For example, as shown in FIG. 18, process 1800 may include an operation 1802, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of SOIs (e.g., SOIs 1-N) may be produced or otherwise obtained. At operation 1804, each calibration regulator of each one of the SOIs may be configured to apply global calibration (e.g., global calibration as may be developed by any suitable process, such as by process 1500 of FIG. 15). For example, an appropriate global calibration may be applied to all the SOIs (e.g., in the factory before providing to an end user), such as by defining a calibration regulator 950 for each of the SOIs (e.g., on each SOI or in the cloud) and configuring each one of the calibration regulators to apply that global calibration. At operation 1806, based on any suitable information (e.g., based on the onboarding of operation 1802 or otherwise (e.g., if the customer has access to one or more reference monitors), collocation may be recommended to the customer and process 1800 may advance from operation 1806 to operation 1808. At operation 1808, collocation-based calibrations may be developed and configured for each SOI (or at least each SOI of operation 1802 that may not have an accessible reference monitor (e.g., pursuant to operation 1806)), before advancing to operation 1828 For example, for SOI 1, operation 1810 may perform collocation with a reference monitor (see, e.g., operation 1402 of process 1400), then operation 1812 may develop a calibration 1 (e.g., a collocation-based calibration 1) (see, e.g., operation 1404 of process 1400), and then operation 1814 may configure a calibration regulator for SOI 1 to apply collocation-based calibration 1 to any suitable data output by SOI 1 (e.g., raw SOI data or globally calibrated SOI data (e.g., by stacking with global calibration applied to raw SOI data due to the configuration of operation 1804 (see, e.g., process 2100)), where operation 1814 may be operative to selectively choose between applying its calibration to raw or globally calibrated data, or operation 1804 may be alternatively provided between operations 1806 and 1828 so that no globally calibrated data may be available to operation 1814). Additionally, for SOI 2, operation 1816 may perform collocation with a reference monitor (see, e.g., operation 1402 of process 1400), then operation 1818 may develop a calibration 2 (e.g., a collocation-based calibration 2) (see, e.g., operation 1404 of process 1400), and then operation 1820 may configure a calibration regulator for SOI 2 to apply collocation-based calibration 2 to any suitable data output by SOI 2 (e.g., raw SOI data or globally calibrated SOI data (e.g., by stacking with global calibration applied to raw SOI data due to the configuration of operation 1804 (see, e.g., process 2100)), where operation 1820 may be operative to selectively choose between applying its calibration to raw or globally calibrated data, or operation 1804 may be alternatively provided between operations 1806 and 1828 so that no globally calibrated data may be available to operation 1820). Additionally, for SOI N, operation 1822 may perform collocation with a reference monitor (see, e.g., operation 1402 of process 1400), then operation 1824 may develop a calibration N (e.g., a collocation-based calibration N) (see, e.g., operation 1404 of process 1400), and then operation 1826 may configure a calibration regulator for SOI N to apply collocation-based calibration N to any suitable data output by SOI N (e.g., raw SOI data or globally calibrated SOI data (e.g., by stacking with global calibration applied to raw SOI data due to the configuration of operation 1804 (see, e.g., process 2100)), where operation 1826 may be operative to selectively choose between applying its calibration to raw or globally calibrated data, or operation 1804 may be alternatively provided between operations 1806 and 1828 so that no globally calibrated data may be available to operation 1826). Otherwise, process 1800 may advance from operation 1806 to operation 1828, where the SOIs may be moved to the desired monitoring locations (e.g., to the appropriate positions in the field as desired by the customer for end use). After operation 1828, process 1800 may advance to operation 1830, where the calibration regulator(s) of each SOI may correct measurements of the SOI with the applicable configured calibration(s) (e.g., global calibration and/or collocation-based calibration of the calibration regulator).

It is understood that the operations shown in process 1800 of FIG. 18 are only illustrative and that existing operations may be modified or omitted, additional operations may be added, and the order of certain operations may be altered.

### FIG. 19—Use Collocation-Based and/or Global Calibration with Raw Normalization

FIG. 19 is a flowchart of an illustrative process 1900 for using calibration with raw normalization. For example, as shown in FIG. 19, process 1900 may include an operation 1902, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of SOIs (e.g., SOIs 1-N) may be produced or otherwise obtained. At operation 1904, raw normalization may be developed for each of the SOIs (see, e.g., raw normalization as may be developed by any suitable process, such as by process 1600 of FIG. 16). At operation 1906, each calibration regulator of each one of the SOIs may be configured to apply the appropriate raw normalization. At operation 1908, each calibration regulator of each one of the SOIs may be configured to apply global calibration (e.g., global calibration as may be developed by any suitable process, such as by process 1500 of FIG. 15). Operation 1908 may be configured to stack on top of operation 1906 (e.g., a global calibration may be applied to normalized raw data rather than to just actual raw data (e.g., at operation 1916)). At operation 1910, based on any suitable information (e.g., based on the onboarding of operation 1902 or otherwise (e.g., if the customer has access to one or more reference monitors), collocation may be recommended to the customer and process 1900 may advance from operation 1910 to operation 1912. At operation 1912, collocation-based calibrations may be developed and configured for each SOI (see, e.g., operations 1810-1826, which may also be described by process 1400 of FIG. 14), before advancing to operation 1914. In some embodiments, operation 1912 may be done on normalized raw data rather than actual raw data, where such normalized raw data may be globally calibrated or not (e.g., if operation 1908 is alternatively positioned between operation 1910 and operation 1914) Otherwise, process 1900 may advance from operation 1910 to operation 1914, where the SOIs may be moved to the desired monitoring locations (e.g., to the appropriate positions in the field as desired by the customer for end use). After operation 1914, process 1900 may advance to operation 1916, where the calibration regulator(s) of each SOI may correct measurements of the SOI with the applicable configured calibration(s) (e.g., global calibration and/or collocation-based calibration of the calibration regulator) and the applicable raw normalization(s). Process 1900 may be similar to process 1800 but may improve accuracy further (e.g., using normalized data).

It is understood that the operations shown in process 1900 of FIG. 19 are only illustrative and that existing operations may be modified or omitted, additional operations may be added, and the order of certain operations may be altered.

### FIG. 20—Use Global Calibrations With Global Calibration Normalization

FIG. 20 is a flowchart of an illustrative process 2000 for using global calibrations with global calibration normalization. For example, as shown in FIG. 20, process 2000 may include an operation 2002, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of SOIs (e.g., SOIs 1-N) may be produced or otherwise obtained. At operation 2004, each calibration regulator of each one of the SOIs may be configured to apply global calibration (e.g., global calibration as may be developed by any suitable process, such as by process 1500 of FIG. 15). At operation 2006, global calibration normalization may be developed for each of the SOIs (see, e.g., global calibration normalization as may be developed by any suitable process, such as by process 1700 of FIG. 17). At operation 2008, each calibration regulator of each one of the SOIs may be configured to apply the appropriate global calibration normalization. At operation 2010, the SOIs may be moved to the desired monitoring locations (e.g., to the appropriate positions in the field as desired by the customer for end use). After operation 2010, process 2000 may advance to operation 2012, where the calibration regulator(s) of each SOI may correct measurements of the SOI with the applicable configured calibration(s) (e.g., global calibration of the calibration regulator) and the applicable global calibration normalization(s). Operation 2008 may be configured to stack on top of operation 2004 (e.g., a global calibration normalization may be applied to globally calibrated raw data (e.g., at operation 2012)).

It is understood that the operations shown in process 2000 of FIG. 20 are only illustrative and that existing operations may be modified or omitted, additional operations may be added, and the order of certain operations may be altered.

### FIG. 21—Global Calibration Scaling To Reference

FIG. 21 is a flowchart of an illustrative process 2100 for using global calibration scaling to reference. For example, as shown in FIG. 21, process 2100 may include an operation 2102, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of SOIs (e.g., SOIs 1-N) may be produced or otherwise obtained. At operation 2104, each calibration regulator of each one of the SOIs may be configured to apply global calibration (e.g., global calibration as may be developed by any suitable process, such as by process 1500 of FIG. 15). At operation 2106, each calibration regulator of each one of the SOIs may be configured to correct SOI measurements with global calibration. At operation 2108, collocation may be performed (e.g., for some or all of the SOIs). At operation 2110, for each SOI, a simple linear regression may be performed between its global calibrated measurements and the reference measurements (e.g., from one or more reference monitors of the collocation) to determine slope and intercept. At operation 2112, for each SOI, the slope and intercept may be recorded as the global calibration scaling for the SOI. At operation 2114, each calibration regulator of each one of the SOIs may be configured to apply global calibration scaling. At operation 2116, the SOIs may be moved to the desired monitoring locations (e.g., to the appropriate positions in the field as desired by the customer for end use). Then, at operation 2118, the calibration regulator(s) of each SOI may correct measurements of the SOI with the applicable configured calibration(s) (e.g., global calibration of the calibration regulator) and the applicable global calibration scaling(s). Operation 2114 may be configured to stack on top of operation 2104 (e.g., a global calibration scaling may be applied to globally calibrated raw data (e.g., at operation 2118)). For example, stacking or layering may be the application of one calibration or normalization or scaling on another (e.g., the output of one may be the input to another).

It is understood that the operations shown in process 2100 of FIG. 21 are only illustrative and that existing operations may be modified or omitted, additional operations may be added, and the order of certain operations may be altered.

### FIG. 22—Dynamic Calibration (Type 1)

FIG. 22 is a flowchart of an illustrative process 2200 for dynamic calibration. For example, as shown in FIG. 22, process 2200 may include an operation 2202, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of SOIs (e.g., SOIs 1-N) may be produced or otherwise obtained. At operation 2204, each calibration regulator of each one of the SOIs may be configured to apply global calibration (e.g., global calibration as may be developed by any suitable process, such as by process 1500 of FIG. 15). At operation 2206, global calibration normalization may be developed for each of the SOIs (see, e.g., global calibration normalization as may be developed by any suitable process, such as by process 1700 of FIG. 17). At operation 2208, each calibration regulator of each one of the SOIs may be configured to apply the appropriate global calibration normalization. At operation 2210, the calibration regulator(s) of each SOI may correct measurements of the SOI with the applicable configured calibration(s) (e.g., global calibration of the calibration regulator) and the applicable global calibration normalization(s). At operation 2212, collocation may be performed for at least one or some of the SOIs (e.g., SOI 1 alone, SOIs 1 to J, etc.). At operation 2214, for each SOI of operation 2212, a simple linear regression may be performed between its global calibrated and normalized measurements and the reference measurements (e.g., from one or more reference monitors of the collocation) to determine slope and intercept. At operation 2216, for an SOI chosen between the SOI(s) of operation 2212 (e.g., the only or any suitable one of the SOIs, which may be multiple SOIs for redundancy and/or back-up purposes), slope and intercept may be recorded as global calibration scaling for all SOIs. At operation 2218, each calibration regulator of each one of the SOIs may be configured to apply that global calibration scaling. At operation 2220, the remaining SOIs (e.g., SOIs 2 to N, SOIs J+1 to N, etc.) may be moved to the desired monitoring location (e.g., to the appropriate positions in the field as desired by the customer for end use). Then, at operation 2222, the calibration regulator(s) of each SOI may correct measurements of the SOI with the applicable configured calibration(s) (e.g., global calibration of the calibration regulator), the applicable global calibration normalization, and the applicable global calibration scaling(s). Operation 2208 may be configured to stack on top of operation 2204 (e.g., a global calibration normalization may be applied to globally calibrated raw data (e.g., at operation 2118)) and/or operation 2218 may be configured to stack on top of operation 2208 (e.g., a global calibration scaling may be applied to globally calibrated raw data that has been global calibration normalized (e.g., at operation 2118)). After operation 2222, process 2200 may wait for any suitable period of time at operation 2224 and may then advance to operation 2226. At operation 2226, it may be determined whether a dynamic update is desired. If no, process 2200 may return to operation 2222, otherwise process 2200 may advance to operation 2228 where new data collected by the SOI(s) of operation 2212 and any reference monitor(s) of the collocation may be added to the collocation data and, optionally, any suitable older collocation data may be removed from the collocation data as process 2200 may continue by readvancing through operations 2214-2224. Therefore, because normalization may be carried out on all SOIs (e.g., global normalization of operation 2208), the system may be enabled to transfer the scaling of one SOI to all the other SOIs (e.g., through operations 2212-2222). Operation 2214 may involve two or more SOIs in order to provide some redundancy to the system (e.g., in case one of the SOIs breaks down over time), but only one may be used in some embodiments. Dynamic updating may be enabled by periodically running operation 2226 and then optionally running operation 2228 and re-running the scaling of operations 2214-2222. For example, operation 2226 may include comparing the performance of the chosen SOI of operation 2216 to the performance of the reference monitor of the collocation at some frequency (e.g., at a time period of operation 2224 (e.g., 3 months or any other suitable period) after the initial iteration of operation 2222) to determine if there has been any slip in the collocation (e.g., between the Summer and Fall or the Fall and Winter seasons, etc.) using any suitable technique(s) (e.g., RMSE, R², etc.). If so, operations 2228 and 2214-2222 may be carried out to rerun the scaling and replace the old scaling data with the new scaling data.

It is understood that the operations shown in process 2200 of FIG. 22 are only illustrative and that existing operations may be modified or omitted, additional operations may be added, and the order of certain operations may be altered.

### FIG. 23—Dynamic Calibration (Type 2)

FIG. 23 is a flowchart of an illustrative process 2300 for dynamic calibration. For example, as shown in FIG. 23, process 2300 may include an operation 2302, where a customer may be onboarded by any suitable entity (e.g., an entity responsible for manufacturing and/or calibrating a sensor prior to the sensor being provided to an end user), where the customer and entity may align on air quality monitoring goals and define a calibration strategy (e.g., using any suitable communication technique(s)). During the on-boarding, any suitable number N of SOIs (e.g., SOIs 1-N) may be produced or otherwise obtained. At operation 2304, raw normalization may be developed for each of the SOIs (see, e.g., raw normalization as may be developed by any suitable process, such as by process 1600 of FIG. 16). At operation 2306, each calibration regulator of each one of the SOIs may be configured to apply the appropriate raw normalization. At operation 2308, the calibration regulator(s) of each SOI may correct raw measurements of the SOI with the applicable configured raw normalization(s). At operation 2310, any suitable collocation-based calibration may be developed (see, e.g., operation 1912 of process 1900) for at least one or some of the SOIs (e.g., SOI 1, SOIs 1 to J, etc.) using normalized raw measurements. At operation 2312, one of the collocation-based calibration(s) may be chosen and each calibration regulator of each one of the SOIs may be configured to apply the chosen collocation-based calibration. At operation 2314, the remaining SOIs (e.g., SOIs 2 to N, SOIs J+1 to N, etc.) may be moved to the desired monitoring location (e.g., to the appropriate positions in the field as desired by the customer for end use). Then, at operation 2316, the calibration regulator(s) of each SOI may correct measurements of the SOI with the applicable configured calibration(s) (e.g., collocation-based calibration of the calibration regulator) and the applicable raw normalization. After operation 2316, process 2300 may wait for any suitable period of time at operation 2318 and may then advance to operation 2320. At operation 2320, it may be determined whether a dynamic update is desired. If no, process 2300 may return to operation 2316, otherwise process 2300 may advance to operation 2322 where new data collected by the at least one SOI of operation 2310 and any reference monitor(s) of the collocation may be added to the collocation data and, optionally, any suitable older collocation data may be removed from the collocation data as process 2300 may continue by readvancing through operations 2310-2316. Therefore, because normalization may be carried out on all SOIs (e.g., raw normalization of operation 2306), the system may be enabled to transfer the collocation-based calibration of one SOI to all the other SOIs (e.g., through operations 2310-2316). Operation 2310 may involve two or more SOIs in order to provide some redundancy to the system (e.g., in case one of the SOIs breaks down over time), but only one may be used in some embodiments. Dynamic updating may be enabled by periodically running operation 2320 and then optionally running operation 2322 and re-running the collocation-based calibration of operations 2310-2316. For example, operation 2320 may include comparing the performance of the chosen SOI of operation 2312 to the performance of the reference monitor of the collocation at some frequency (e.g., at a time period of operation 2318 (e.g., 3 months or any other suitable period) after the initial iteration of operation 2316) to determine if there has been any slip in the collocation (e.g., between the Summer and Fall or the Fall and Winter seasons, etc.) using any suitable technique(s) (e.g., RMSE, R², etc.). If so, operations 2322 and 2310-2316 may be carried out to rerun the collocation-based calibration and replace the old collocation-based calibration data with the new collocation-based calibration data.

It is understood that the operations shown in process 2300 of FIG. 23 are only illustrative and that existing operations may be modified or omitted, additional operations may be added, and the order of certain operations may be altered.

### Other Apparatus, Systems, Methods, and Non-Transitory Computer-Readable Storage Media

Therefore, while process 1800 may use global calibration and/or collocation-based calibration, process 1900 may use raw normalization and global calibration and/or collocation-based calibration, process 2000 may use global calibration and global calibration normalization, process 2100 may use global calibration and global calibration scaling, process 2200 may use global calibration and global calibration normalization and global calibration scaling, and process 2300 may use raw normalization and collocation-based calibration, any suitable combinations and orderings of calibration(s), normalization(s), and/or scaling(s) (e.g., any suitable layering or stacking or hybrid approaches) may be utilized by various methods of a system of the disclosure, one or more of which may enable dynamic updates (e.g., based on ongoing collocation data).

A method is provided that may include accessing collocation data for each one of a plurality of collocations (see, e.g., operation 1502), wherein the accessed collocation data for each collocation of the plurality of collocations may include: sensor data collected from a sensor of the collocation over a collocation period of time of the collocation; and reference monitor data collected from a reference monitor of the collocation over the collocation period of time of the collocation; and wherein, for each collocation of the plurality of collocations, the sensor of the collocation was collocated with the reference monitor of the collocation during the collocation period of time of the collocation at a location of the collocation. The method may also include combining the accessed collocation data from each one the plurality of collocations into global collocation data (see, e.g., operations 1510-1516), developing a global calibration on the global collocation data (see, e.g., operation 1518), and configuring a calibration regulator for a sensor of interest (“SOI”) to apply the developed global calibration (see, e.g., operation 1804). In some embodiments, the SOI is not a sensor of any collocation of the plurality of collocations (see, e.g., operation 1502). In some embodiments, the sensor of each collocation of the plurality of collocations is the same particular type of sensor (see, e.g., operation 1502). In some embodiments, the location of a first collocation of the plurality of collocations is different than the location of a second collocation of the plurality of collocations (see, e.g., operation 1502). In some embodiments, the collocation period of time of a first collocation of the plurality of collocations is different than the collocation period of time of a second collocation of the plurality of collocations (see, e.g., operation 1502). In some embodiments, the location of a first collocation of the plurality of collocations is different than the location of a second collocation of the plurality of collocations; and the collocation period of time of a first collocation of the plurality of collocations is different than the collocation period of time of a second collocation of the plurality of collocations (see, e.g., operation 1502). In some embodiments, the method may further include positioning the SOI in an SOI monitoring position (see, e.g., operation 1828); after the positioning, obtaining an SOI sensor reading from the SOI (see, e.g., operation 1830); and correcting the obtained SOI sensor reading with the developed global calibration using the configured calibration regulator for the SOI (see, e.g., operation 1830). In some such further embodiments, the positioning may include positioning the SOI in the SOI monitoring position to be collocated with an SOI reference monitor (see, e.g., operation 2108); the obtaining may include obtaining the SOI sensor reading from the SOI and obtaining a reference measurement from the SOI reference monitor (see, e.g., operation 2110); and the method may further include performing a linear regression between the corrected SOI sensor reading and the obtained reference measurement (see, e.g., operation 2110). In some such further embodiments, the method may further include developing a global calibration scaling based on the performed linear regression (see, e.g., operation 2112). In some such further embodiments, the method may further include moving the SOI to a different SOI monitoring position after the obtaining (see, e.g., operation 2116); after the moving, obtaining another SOI sensor reading from the SOI (see, e.g., operation 2116); and correcting the obtained other SOI sensor reading with the developed global calibration and with the developed global calibration scaling (see, e.g., operation 2118). In some such further embodiments, the method may further include further configuring the calibration regulator for the SOI to apply the developed global calibration scaling (see, e.g., operation 2118). In some such further embodiments, the method may further include moving the SOI to a different SOI monitoring position after the obtaining (see, e.g., operation 2116); after the moving, obtaining another SOI sensor reading from the SOI (see, e.g., operation 2116); and correcting the obtained other SOI sensor reading with the developed global calibration and with the developed global calibration scaling using the further configured calibration regulator for the SOI (see, e.g., operation 2118). In some embodiments, the method may also include, prior to the configuring, providing a plurality of SOIs that includes the SOI and at least one other SOI (see, e.g., operation 2202), wherein the configuring includes configuring a calibration regulator for each SOI of the plurality of SOIs to apply the developed global calibration (see, e.g., operation 2204). In some such further embodiments, the method may also include positioning the SOI in an SOI monitoring position that is collocated with an SOI reference monitor (see, e.g., operation 2212); after the positioning, obtaining an SOI sensor reading from the SOI and obtaining a reference measurement from the SOI reference monitor (see, e.g., operation 2212); correcting the obtained SOI sensor reading with the developed global calibration using the configured calibration regulator for the SOI (see, e.g., operation 2212); and performing a linear regression between the corrected SOI sensor reading and the obtained reference measurement. In some such further embodiments, the method may also include developing a global calibration scaling based on the performed linear regression (see, e.g., operation 2112). In some such further embodiments, the method may also include placing the at least one other SOI in a different SOI monitoring position than the SOI monitoring position (see, e.g., operation 2220); after the placing, obtaining another SOI sensor reading from the at least one other SOI (see, e.g., operation 2222); and correcting the obtained other SOI sensor reading with the developed global calibration and with the developed global calibration scaling (see, e.g., operation 2222). In some such further embodiments, the method may also include further configuring the calibration regulator for the at least one other SOI to apply the developed global calibration scaling (see, e.g., operation 2218). In some such further embodiments, the method may also include placing the at least one other SOI in a different SOI monitoring position than the SOI monitoring position (see, e.g., operation 2220); after the placing, obtaining another SOI sensor reading from the at least one other SOI (see, e.g., operation 2222); and correcting the obtained other SOI sensor reading with the developed global calibration and with the developed global calibration scaling using the further configured calibration regulator for the at least one other SOI (see, e.g., operation 2222).

A non-transitory computer-readable storage medium may be provided storing at least one program, the at least one program including instructions, which, when executed by at least one processor of an electronic subsystem, may cause the at least one processor to: access collocation data for each one of a plurality of collocations (see, e.g., operation 1502), wherein: the accessed collocation data for each collocation of the plurality of collocations may include: sensor data collected from a sensor of the collocation over a collocation period of time of the collocation; and reference monitor data collected from a reference monitor of the collocation over the collocation period of time of the collocation; and for each collocation of the plurality of collocations, the sensor of the collocation was collocated with the reference monitor of the collocation during the collocation period of time of the collocation at a location of the collocation; combine the accessed collocation data from each one the plurality of collocations into global collocation data (see, e.g., operations 1510-1516); develop a global calibration on the global collocation data (see, e.g., operation 1518); and configure a calibration regulator for a sensor of interest (“SOI”) to apply the developed global calibration (see, e.g., operation 1804).

A system is provided that may include a memory component; a communications component; and a processor component configured to: access collocation data for each one of a plurality of collocations using the communications component (see, e.g., operation 1502), wherein: the accessed collocation data for each collocation of the plurality of collocations may include: sensor data collected from a sensor of the collocation over a collocation period of time of the collocation; and reference monitor data collected from a reference monitor of the collocation over the collocation period of time of the collocation; and for each collocation of the plurality of collocations, the sensor of the collocation was collocated with the reference monitor of the collocation during the collocation period of time of the collocation at a location of the collocation; combine the accessed collocation data from each one the plurality of collocations into global collocation data (see, e.g., operations 1510-1516); develop a global calibration on the global collocation data (see, e.g., operation 1518); and configure, in the memory component, a calibration regulator for a sensor of interest (“SOI”) to apply the developed global calibration (see, e.g., operation 1804).

A compact sensor apparatus may be provided that may include a power module configured to supply a reliable power supply from a primary power source supplemented by a secondary power source, a sensor module configured to monitor a gas for one or more characteristics, a communication module configured to establish a wireless communication channel over a network with a host, a controller configured to manage the sensor module and to send measurement data to the host by way of the wireless communication channel, a printed circuit board configured to interconnect the power module, the reliable power supply, the controller, and the communication module, and an enclosure configured to house the printed circuit board, the power module, the sensor module, the communication module, and the controller. In some embodiments, the gas may be ambient air. In some embodiments, the power module may include a power management circuit configured to monitor electrical load and maintain the reliable power supply by selectively supplying supplemental power from a battery in response to the primary power source supplying power below a threshold. In some embodiments, the primary power source may include one of a wired power supply and a solar power module and the secondary power source may include a battery. In some embodiments, the enclosure may include a body and a lid, the lid may include a gimbal fastener configured to connect a solar power module to the lid.

A system may be provided that may include an interchangeable sensor module configured to monitor an air sample for one or more characteristics, an enclosure including the interchangeable sensor module, a power module configured to supply power, a communication module configured to establish a wireless communication channel over a network with a host, a controller configured to manage the interchangeable sensor module and to send measurement data to the host by way of the wireless communication channel, and a universal mount configured to mount the enclosure in a plurality of mounting configurations. In some embodiments, the enclosure may be configured to atmospherically isolate the interchangeable sensor module from the power module, the communication module, and the controller; the system further may include removable fasteners configured to engage the enclosure and the interchangeable sensor module. In some embodiments, the interchangeable sensor module may include an inlet port and an outlet port and wherein the enclosure may include a pair of openings, each opening sized and positioned to align with one of the inlet port and the outlet port. In some embodiments, the power module may include a solar power module configured to supply current as a primary power source and a battery configured to selectively supply current as the primary power source and to supplement power supplied by the solar power module. In some embodiments, the enclosure may include a battery compartment configured to receive the battery and to integrate the battery with the system and a gimbal fastener configured to engage the solar power module and to integrate the solar power module with the system. In some embodiments, the enclosure may include two or more openings, a body, one or more seals, and a lid, the lid fastened to the body by way of removable fasteners; wherein the seals, openings and lid are configured to engage the body to provide a liquid ingress protection rating greater than four. In some embodiments, the enclosure may include a body and a lid, the lid permanently connected to the body, the enclosure including an opening and a door, the door configured to seal the opening from moisture ingress when the door is closed, the opening sized to slidably accept the interchangeable sensor module. The system may further include a printed circuit board configured to connect the power module, the communication module, and the controller, the printed circuit board further including an input/output connector configured to couple the interchangeable sensor module to the printed circuit board. In some embodiments, the system may further include a second interchangeable sensor module configured to monitor one or more environmental characteristics, the second interchangeable sensor module configured to sit within the enclosure and configured to removably couple to the controller and the power module.

A method may be provided that may include placing a first sensor node near a reference monitor within a region; placing a plurality of sensor nodes at various locations within the region; gathering measurement data from the first sensor node, the reference monitor, and the plurality of sensor nodes; determining a calibration profile for each of the first sensor node and the plurality of sensor nodes based on measurement data from the reference monitor and the first sensor node; and applying the calibration profile for each of the first sensor node and the plurality of sensor nodes to measurement data from each of the first sensor node and the plurality of sensor nodes to obtain calibrated measurement data for each of the first sensor node and the plurality of sensor nodes. In some embodiments, applying the calibration profile may include wirelessly communicating a calibration profile to each of the first sensor node and the plurality of sensor nodes (and wherein the first sensor node and reference monitor may include a co-location pair). In some embodiments, determining the calibration profile may include determining a calibration model for the first sensor node and determining a set of calibration constants for the first sensor node. In some embodiments, applying the calibration profile may include applying the calibration model to measurement data for one or more of the plurality of sensor nodes to generate calibrated measurement data. In some embodiments, determining the calibration profile and applying the calibration profile for each of the first sensor node and the plurality of sensor nodes may be performed at a host in communication with the first sensor node, the reference monitor, and the plurality of sensor nodes by way of a network. In some embodiments, applying the calibration profile for each of the first sensor node and the plurality of sensor nodes may further include communicating the calibration profile for each of the first sensor node and the plurality of sensor nodes to each of the first sensor node and the plurality of sensor nodes such that the first sensor node and the plurality of sensor nodes apply the calibration profile to generate calibrated measurement data.

A method for obtaining information about a system may be provided, wherein the system includes a data management platform, a first reference monitor positioned at a first reference monitor location, a first sensor node positioned at a first sensor node location that is within a distance limit of the first reference monitor location, a second reference monitor positioned at a second reference monitor location, a second sensor node positioned at a second sensor node location that is within the distance limit of the second reference monitor location, and a third sensor node positioned at a third sensor node location that is outside the distance limit of the first reference monitor location and that is outside the distance limit of the second reference monitor location, the method including: selecting, using the data management platform, a particular calibration profile for the third sensor node from a plurality of calibration profiles that may include a first calibration profile associated with the first sensor node and a second calibration profile associated with the second sensor node; gathering, using the third sensor node, measurement data for the third sensor node location; and applying the selected particular calibration profile to the gathered measurement data for the third sensor node location to obtain calibrated measurement data for the third sensor node location. In some embodiments, the gathering occurs prior to the selecting. In some embodiments, the method may further include receiving the gathered measurement data for the third sensor node location at the data management platform from the third sensor node, wherein the applying may include applying, using the data management platform, the selected particular calibration profile to the received gathered measurement data for the third sensor node location to obtain the calibrated measurement data for the third sensor node location. In some embodiments, the method may further include receiving the gathered measurement data for the third sensor node location at the data management platform from the third sensor node, wherein the applying may include applying, using the data management platform, the selected particular calibration profile to the received gathered measurement data for the third sensor node location to obtain the calibrated measurement data for the third sensor node location. In some embodiments, the selecting occurs prior to the gathering. In some embodiments, the method may further include receiving the selected particular calibration profile at the third sensor node from the data management platform, wherein the applying may include applying, using the third sensor node, the received selected particular calibration profile to the gathered measurement data for the third sensor node location to obtain the calibrated measurement data for the third sensor node location. In some embodiments, the method may further include receiving the selected particular calibration profile at the third sensor node from the data management platform, wherein the applying may include applying, using the third sensor node, the received selected particular calibration profile to the gathered measurement data for the third sensor node location to obtain the calibrated measurement data for the third sensor node location. In some embodiments, the magnitude of the distance limit is defined such that, if the distance between a sensor node and a reference monitor is lower than the magnitude of the distance limit, then the reference monitor and the sensor node are accurately considered to be exposed to the same concentration of air pollutants. In some embodiments, the selecting may include selecting the first calibration profile as the particular calibration profile for the third sensor node when the third sensor node location is closer to the first reference monitor location than to the second reference monitor location; and the selecting may include selecting the second calibration profile as the particular calibration profile for the third sensor node when the third sensor node location is closer to the second reference monitor location than to the first reference monitor location. In some embodiments, the selecting may include selecting the particular calibration profile for the third sensor node based on at least one of: land use information; meteorological information; or traffic information.

A method for generating information about a system may be provided, wherein the system may include a data management platform, a first reference monitor positioned at a first reference monitor location, and a first sensor node positioned at a first sensor node location that is within a distance limit of the first reference monitor location, the method including: periodically gathering over a time interval, using the first reference monitor, measurement data for the first reference monitor location; periodically gathering over the time interval, using the first sensor node, measurement data for the first sensor node location; and calculating, using the data management platform, a calibration profile for the first sensor node based on: the gathered measurement data for the first reference monitor location; and the gathered measurement data for the first sensor node location. In some embodiments, the first reference monitor may include an air quality monitoring station operated by a governmental agency and with measurement accuracy higher than the accuracy of the first sensor node. In some embodiments, the calculating may include determining a plurality of calibration constants of the calibration profile by fitting a calibration model of the calibration profile to the gathered measurement data for the first reference monitor location and to the gathered measurement data for the first sensor node location. In some embodiments, the method may also include applying the calculated calibration profile to measurement data gathered by a second sensor node positioned at a second sensor node location to obtain calibrated measurement data for the second sensor node location, wherein the applying may include multiplying the gathered measurement data for the second sensor node location with a calibration constant of the plurality of calibration constants of the calculated calibration profile. In some embodiments, the applying further includes multiplying another calibration constant of the plurality of calibration constants with one of a temperature measurement of the system or a humidity measurement of the system. In some embodiments, a calibration constant of the plurality of calibration constants may include an offset coefficient. In some embodiments, a calibration constant of the plurality of calibration constants may include a bias coefficient. In some embodiments, a first calibration constant of the plurality of calibration constants may include an offset coefficient; and a second calibration constant of the plurality of calibration constants may include a bias coefficient. In some embodiments, the calibration model may be a linear model. In some embodiments, the first reference monitor may be operated by a governmental agency. In some embodiments, the measurement accuracy of data gathering by the first reference monitor may be higher than the measurement accuracy of data gathering by the first sensor node. In some embodiments, the system further may include a second reference monitor positioned at a second reference monitor location, a second sensor node positioned at a second sensor node location that is within the distance limit of the second reference monitor location, and a third sensor node positioned at a third sensor node location that is outside the distance limit of the first reference monitor location and that is outside the distance limit of the second reference monitor location, the method further including: selecting, using the data management platform, a particular calibration profile for the third sensor node from a plurality of calibration profiles that includes the first calibration profile for the first sensor node and a second calibration profile for the second sensor node; gathering, using the third sensor node, measurement data for the third sensor node location; and applying the selected particular calibration profile to the gathered measurement data for the third sensor node location to obtain calibrated measurement data for the third sensor node location. In some embodiments, the gathering the measurement data for the third sensor node location occurs prior to the selecting. In some embodiments, the method further includes receiving the gathered measurement data for the third sensor node location at the data management platform from the third sensor node, wherein the applying may include applying, using the data management platform, the selected particular calibration profile to the received gathered measurement data for the third sensor node location to obtain the calibrated measurement data for the third sensor node location. In some embodiments, the method may include receiving the gathered measurement data for the third sensor node location at the data management platform from the third sensor node, wherein the applying includes applying, using the data management platform, the selected particular calibration profile to the received gathered measurement data for the third sensor node location to obtain the calibrated measurement data for the third sensor node location. In some embodiments, the selecting occurs prior to the gathering the measurement data for the third sensor node location. In some embodiments, the method may further include receiving the selected particular calibration profile at the third sensor node from the data management platform, wherein the applying includes applying, using the third sensor node, the received selected particular calibration profile to the gathered measurement data for the third sensor node location to obtain the calibrated measurement data for the third sensor node location. In some embodiments, the method may further include receiving the selected particular calibration profile at the third sensor node from the data management platform, wherein the applying includes applying, using the third sensor node, the received selected particular calibration profile to the gathered measurement data for the third sensor node location to obtain the calibrated measurement data for the third sensor node location. In some embodiments, the selecting may include selecting the first calibration profile as the particular calibration profile for the third sensor node when the third sensor node location is closer to the first reference monitor location than to the second reference monitor location; and the selecting may include selecting the second calibration profile as the particular calibration profile for the third sensor node when the third sensor node location is closer to the second reference monitor location than to the first reference monitor location. In some embodiments, the selecting may include selecting the particular calibration profile for the third sensor node based on at least one of: land use information; meteorological information; or traffic information. In some embodiments, the magnitude of the distance limit may be defined such that, if the distance between a sensor node and a reference monitor is lower than the magnitude of the distance limit, then the reference monitor and the sensor node are accurately considered to be exposed to the same concentration of air pollutants. In some embodiments, the method may further include, after the calculating, applying the calculated calibration profile to measurement data gathered by the first sensor node to obtain calibrated measurement data for the first sensor node location. In some embodiments, the applying may include multiplying the gathered measurement data for the first sensor node location with a calibration constant of the plurality of calibration constants of the calculated calibration profile. In some embodiments, the applying may further include multiplying another calibration constant of the plurality of calibration constants with one of a temperature measurement of the system or a humidity measurement of the system. In some embodiments, the method may further include wirelessly communicating the calculated calibration profile to the first sensor node. In some embodiments, the method may further include, prior to the calculating, wirelessly communicating the gathered measurement data for the first sensor node location to the data management platform. In some embodiments, the method may further include, prior to the calculating, wirelessly communicating the gathered measurement data for the first sensor node location to the data management platform.

A method for calibrating air quality sensors as described herein.

A system for calibrating air quality sensors as described herein may be provided.

A non-transitory computer-readable storage medium storing at least one program, the at least one program including instructions, which, when executed by at least one processor of an electronic subsystem, cause the at least one processor to calibrate air quality sensors as described herein may be provided.

A method for generating information about a system may be provided, wherein the system includes a data management platform, a first reference gas monitor positioned at a first reference gas monitor location, a first gas sensor node, and a second gas sensor node, the method may include: positioning the first gas sensor node at a first gas sensor node location that is within a distance limit of the first reference gas monitor location; positioning the second gas sensor node at a second gas sensor node location, wherein the second gas sensor node location is further away from the first reference gas monitor location than the first gas sensor node location is from the first reference gas monitor location; periodically gathering over a time interval, using the first reference gas monitor, gas quality measurement data for the first reference gas monitor location; periodically gathering over the time interval, using the first gas sensor node positioned at the first gas sensor node location, gas quality measurement data for the first gas sensor node location; calculating, using the data management platform, a calibration profile for the first gas sensor node based on: the gathered gas quality measurement data for the first reference gas monitor location; and the gathered gas quality measurement data for the positioned first gas sensor node location; applying the calculated calibration profile to gas quality measurement data gathered by the second gas sensor node positioned at the second gas sensor node location to obtain calibrated gas quality measurement data for the second gas sensor node location; and storing the calibrated gas quality measurement data for the second gas sensor node location in storage media of the system. In some embodiments, the first reference gas monitor may include an air quality monitoring station operated by a governmental agency and with measurement accuracy higher than the accuracy of the first gas sensor node. In some embodiments, the calculating may include determining a plurality of calibration constants of the calibration profile by fitting a calibration model of the calibration profile to the gathered gas quality measurement data for the first reference gas monitor location and to the gathered gas quality measurement data for the first gas sensor node location. In some embodiments, the applying may include multiplying the gathered gas quality measurement data for the second gas sensor node location with a calibration constant of the plurality of calibration constants of the calculated calibration profile. In some embodiments, the applying may further include multiplying another calibration constant of the plurality of calibration constants with one of a temperature measurement of the system or a humidity measurement of the system. In some embodiments, a calibration constant of the plurality of calibration constants may include an offset coefficient. In some embodiments, a calibration constant of the plurality of calibration constants may include a bias coefficient. In some embodiments, a first calibration constant of the plurality of calibration constants may include an offset coefficient; and a second calibration constant of the plurality of calibration constants may include a bias coefficient. In some embodiments, the calibration model is a linear model. In some embodiments, the first reference gas monitor is operated by a governmental agency. In some embodiments, the measurement accuracy of data gathering by the first reference gas monitor is higher than the measurement accuracy of data gathering by the first gas sensor node.

A method for generating information about a system may be provided, wherein the system includes a data management platform, a first reference monitor positioned at a first reference monitor location, and a first sensor node positioned at a first sensor node location that is within a distance limit of the first reference monitor location, wherein the system further includes a second reference monitor positioned at a second reference monitor location, a second sensor node positioned at a second sensor node location that is within the distance limit of the second reference monitor location, and a third sensor node positioned at a third sensor node location that is outside the distance limit of the first reference monitor location and that is outside the distance limit of the second reference monitor location, the method may include: periodically gathering over a time interval, using the first reference monitor, gas measurement data for the first reference monitor location; periodically gathering over the time interval, using the first sensor node, gas measurement data for the first sensor node location; calculating, using the data management platform, a first calibration profile for the first sensor node based on: the gathered gas measurement data for the first reference monitor location; and the gathered gas measurement data for the first sensor node location; selecting, using the data management platform, a particular calibration profile for the third sensor node from a plurality of calibration profiles that may include the first calibration profile for the first sensor node and a second calibration profile for the second sensor node; gathering, using the third sensor node, gas measurement data for the third sensor node location; applying the selected particular calibration profile to the gathered gas measurement data for the third sensor node location to obtain calibrated measurement data for the third sensor node location; and storing the calibrated measurement data for the third sensor node location in storage media. In some embodiments, the gathering the gas measurement data for the third sensor node location occurs prior to the selecting. In some embodiments, the method may further include receiving the gathered gas measurement data for the third sensor node location at the data management platform from the third sensor node, wherein the applying may include applying, using the data management platform, the selected particular calibration profile to the received gathered gas measurement data for the third sensor node location to obtain the calibrated measurement data for the third sensor node location. In some embodiments, the method may further include receiving the gathered gas measurement data for the third sensor node location at the data management platform from the third sensor node, wherein the applying may include applying, using the data management platform, the selected particular calibration profile to the received gathered gas measurement data for the third sensor node location to obtain the calibrated measurement data for the third sensor node location. In some embodiments, the selecting may occur prior to the gathering the gas measurement data for the third sensor node location. In some embodiments, the method may further include receiving the selected particular calibration profile at the third sensor node from the data management platform, wherein the applying may include applying, using the third sensor node, the received selected particular calibration profile to the gathered gas measurement data for the third sensor node location to obtain the calibrated measurement data for the third sensor node location. In some embodiments, the method may further include receiving the selected particular calibration profile at the third sensor node from the data management platform, wherein the applying may include applying, using the third sensor node, the received selected particular calibration profile to the gathered gas measurement data for the third sensor node location to obtain the calibrated measurement data for the third sensor node location. In some embodiments, the selecting may include selecting the first calibration profile as the particular calibration profile for the third sensor node when the third sensor node location is closer to the first reference monitor location than to the second reference monitor location; and the selecting may include selecting the second calibration profile as the particular calibration profile for the third sensor node when the third sensor node location is closer to the second reference monitor location than to the first reference monitor location. In some embodiments, the selecting may include selecting the particular calibration profile for the third sensor node based on at least one of: land use information; meteorological information; or traffic information. In some embodiments, the magnitude of the distance limit may be defined such that, if the distance between a gas sensor node and a reference gas monitor is lower than the magnitude of the distance limit, then the reference gas monitor and the gas sensor node are accurately considered to be exposed to the same concentration of air pollutants. In some embodiments, the method may further include, after the calculating, applying the calculated calibration profile to gas quality measurement data gathered by the first gas sensor node to obtain calibrated gas quality measurement data for the first gas sensor node location. In some embodiments, the applying may include multiplying the gathered gas quality measurement data for the first gas sensor node location with a calibration constant of the plurality of calibration constants of the calculated calibration profile. In some embodiments, the applying may further include multiplying another calibration constant of the plurality of calibration constants with one of a temperature measurement of the system or a humidity measurement of the system. In some embodiments, the method may further include wirelessly communicating the calculated calibration profile to the first gas sensor node. In some embodiments, the method may further include, prior to the calculating, wirelessly communicating the gathered gas quality measurement data for the first gas sensor node location to the data management platform. In some embodiments, the method may further include, prior to the calculating, wirelessly communicating the gathered gas quality measurement data for the first gas sensor node location to the data management platform. In some embodiments, the second gas sensor node location is not within the distance limit of the first reference gas monitor location. In some embodiments, the magnitude of the distance limit may be defined such that, if the distance between a gas sensor node and a reference gas monitor is lower than the magnitude of the distance limit, then the reference gas monitor and the gas sensor node are accurately considered to be exposed to the same concentration of air pollutants.

A method for generating information about a system may be provided, wherein the system includes a data management platform, a first reference gas monitor positioned at a first reference monitor location, and a first gas sensor node positioned at a first sensor node location that is within a distance limit of the first reference monitor location, wherein the system further includes a second reference gas monitor positioned at a second reference monitor location, a second gas sensor node positioned at a second sensor node location that is within the distance limit of the second reference monitor location, and a third gas sensor node positioned at a third sensor node location that is not within the distance limit of the first reference monitor location and that is not within the distance limit of the second reference monitor location, the method may include: gathering over a first time interval, using the first reference gas monitor, gas quality measurement data for the first reference monitor location; gathering over the first time interval, using the first gas sensor node, gas quality measurement data for the first sensor node location; calculating a first calibration profile for the first gas sensor node based on the gathered gas quality measurement data for the first reference monitor location; storing the first calibration file in the system; gathering over a second time interval, using the second reference gas monitor, gas quality measurement data for the second reference monitor location; gathering over the second time interval, using the second gas sensor node, gas quality measurement data for the second sensor node location; calculating a second calibration profile for the second gas sensor node based on the gathered gas quality measurement data for the second reference monitor location; storing the second calibration file in the system; selecting a particular calibration profile for the third gas sensor node from the stored first calibration profile for the first gas sensor node and the stored second calibration profile for the second gas sensor node; and applying the selected particular calibration profile to gas quality measurement data for the third sensor node location to obtain calibrated measurement data for the third sensor node location.

### Further Remarks

One, some, or all of the processes described with respect to FIGS. 1-23 and otherwise may each be partially or entirely implemented by software, but may also be implemented in hardware, firmware, or any combination of software, hardware, and firmware. Instructions for performing these processes may also be embodied as machine- or computer-readable code recorded on a machine- or computer-readable medium. In some embodiments, the computer-readable medium may be a non-transitory computer-readable medium. Examples of such a non-transitory computer-readable medium include but are not limited to a read-only memory, a random-access memory, a flash memory, a CD-ROM, a DVD, a magnetic tape, a removable memory card, and a data storage device (e.g., storage subsystem 1316). In other embodiments, the computer-readable medium may be a transitory computer-readable medium. In such embodiments, the transitory computer-readable medium can be distributed over network-coupled computer systems so that the computer-readable code is stored and executed in a distributed fashion. For example, such a transitory computer-readable medium may be communicated from a central network controller device to a router device or from a data device to any network device. Such a transitory computer-readable medium may embody computer-readable code, instructions, data structures, program modules, or other data in a modulated data signal, such as a carrier wave or other transport mechanism, and may include any information delivery media. A modulated data signal may be a signal that has one or more of its characteristics set or changed in such a manner as to encode information in the signal.

Any, each, or at least one module or component or subsystem of the disclosure may be provided as a software construct, firmware construct, one or more hardware components, or a combination thereof. For example, any, each, or at least one module or component or subsystem of any suitable system may be described in the general context of computer-executable instructions, such as program modules, that may be executed by one or more computers or other devices. Generally, a program module may include one or more routines, programs, objects, components, and/or data structures that may perform one or more particular tasks or that may implement one or more particular abstract data types. The number, configuration, functionality, and interconnection of the modules and components and subsystems of system 1 are only illustrative, and that the number, configuration, functionality, and interconnection of existing modules, components, and/or subsystems may be modified or omitted, additional modules, components, and/or subsystems may be added, and the interconnection of certain modules, components, and/or subsystems may be altered.

Implementations within the scope of the present disclosure can be partially or entirely realized using a tangible computer-readable storage medium, or multiple tangible computer-readable storage media of one or more types, encoding one or more instructions. The tangible computer-readable storage medium also can be non-transitory in nature.

At least a portion of one or more of the modules of any suitable system of the disclosure may be stored in or otherwise accessible to a subsystem in any suitable manner (e.g., in storage subsystem 1316). Any or each module of any suitable system of the disclosure may be implemented using any suitable technologies (e.g., as one or more integrated circuit devices), and different modules may or may not be identical in structure, capabilities, and operation. Any or all of the modules or other components of any suitable system of the disclosure may be mounted on an expansion card, mounted directly on a system motherboard, or integrated into a system chipset component (e.g., into a “north bridge” chip). At least a portion of one or more of the modules of any system of the disclosure may be stored in or otherwise accessible any suitable component(s) in any suitable manner. Any or each module of any suitable system of the disclosure may be implemented using any suitable technologies (e.g., as one or more integrated circuit devices), and different modules may or may not be identical in structure, capabilities, and operation. Any or all of the modules or other components of any suitable system of the disclosure may be mounted on an expansion card, mounted directly on a system motherboard, or integrated into a system chipset component (e.g., into a “north bridge” chip).

Any or each module of any suitable system of the disclosure may be a dedicated system implemented using one or more expansion cards adapted for various bus standards. For example, all of the modules may be mounted on different interconnected expansion cards or all of the modules may be mounted on one expansion card. With respect to a system, by way of example only, modules of the system may interface with a motherboard or processor assembly through an expansion slot (e.g., a peripheral component interconnect (“PCI”) slot or a PCI express slot). Alternatively, modules of the system need not be removable but may include one or more dedicated modules that may include memory (e.g., RAM) dedicated to the utilization of the module. In other embodiments, modules of the system may be at least partially integrated into a subsystem. For example, a module of the system may utilize a portion of memory of a subsystem. Any or each module of the system may include its own processing circuitry and/or memory. Alternatively, any or each module of the system may share processing circuitry and/or memory with any other module of the system and/or processor assembly and/or memory assembly of a subsystem.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device (e.g., via one or more wired connections, one or more wireless connections, or any combination thereof).

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including, but not limited to, routines, subroutines, programs, data structures, objects, modules, applications, applets, functions, and/or the like. As recognized by those of skill in the art, details including, but not limited to, the number, structure, sequence, and organization of instructions can vary significantly without varying the underlying logic, function, processing, and output.

While the above discussion primarily may refer to microprocessor or multi-core processors that execute software, one or more implementations may be performed by one or more integrated circuits, such as ASICs or FPGAs. In one or more implementations, such integrated circuits may execute instructions that may be stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software may depend upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Any suitable calibration model may be developed and/or generated for use in evaluating and/or predicting output states. For example, a model may be a learning engine for an experiencing entity, where the learning engine may be operative to use any suitable machine learning (“ML”) (e.g., the system's ability to learn automatically from past events to affect future behavior) to use certain monitored system data for a particular environment (e.g., at a particular time and/or with respect to one or more planned activities) in order to predict, estimate, and/or otherwise generate an output state. For example, the learning engine may include any suitable neural network (e.g., an artificial neural network) that may be initially configured, trained on one or more sets of monitored system data that is associated with known or otherwise determined or confirmed states or data from any suitable sources, and then used to predict further states based on another set of monitored system data.

A neural network or neuronal network or artificial neural network may be hardware-based, software-based, or any combination thereof, such as any suitable model (e.g., an analytical model, a computational model, etc.), which, in some embodiments, may include one or more sets or matrices of weights (e.g., adaptive weights, which may be numerical parameters that may be tuned by one or more learning algorithms or training methods or other suitable processes) and/or may be capable of approximating one or more functions (e.g., non-linear functions or transfer functions) of its inputs. The weights may be connection strengths between neurons of the network, which may be activated during training and/or prediction. A neural network may generally be a system of interconnected neurons that can compute values from inputs and/or that may be capable of machine learning and/or pattern recognition (e.g., due to an adaptive nature). A neural network may use any suitable machine learning techniques to optimize a training process. The neural network may be used to estimate or approximate functions that can depend on a large number of inputs and that may be generally unknown. The neural network may generally be a system of interconnected “neurons” that may exchange messages between each other, where the connections may have numeric weights (e.g., initially configured with initial weight values) that can be tuned based on experience, making the neural network adaptive to inputs and capable of learning (e.g., learning pattern recognition). A suitable optimization or training process may be operative to modify a set of initially configured weights assigned to the output of one, some, or all neurons from the input(s) and/or hidden layer(s). A non-linear transfer function may be used to couple any two portions of any two layers of neurons, including an input layer, one or more hidden layers, and an output (e.g., an input to a hidden layer, a hidden layer to an output, etc.).

Different input neurons of the neural network may be associated with respective different types of monitored system data categories and may be activated by monitored system data of the respective monitored system data categories (e.g., each possible category of monitored system data variable information may be associated with one or more particular respective input neurons of the neural network and monitored system data for the particular monitored system data category may be operative to activate the associated input neuron(s)). The weight assigned to the output of each neuron may be initially configured using any suitable determinations that may be made by a custodian or processor of the model based on the data available to that custodian.

The initial configuring of the learning engine or management model for a particular system (e.g., the initial weighting and arranging of neurons of a neural network of the learning engine) may be done using any suitable data accessible to a custodian of the management model, such as data associated with the configuration of other learning engines of the system (e.g., learning engines or management models for other systems), data associated with the particular system (e.g., initial background data accessible by the model custodian about the particular system composition, location, past uses, and/or the like), data assumed or inferred by the model custodian using any suitable guidance, and/or the like. For example, a model custodian may be operative to capture any suitable initial background data about a particular system in any suitable manner, which may be enabled by any suitable user interface provided to an appropriate subsystem or device accessible to one, some, or each operator or entity with knowledge of the particular system (e.g., a model app or website). The model custodian may provide a data collection portal for enabling any suitable entity to provide initial background data for the particular system. The data may be uploaded in bulk or manually entered in any suitable manner.

A management model custodian may receive not only monitored system data for at least one monitored system data category for a particular system experience but also a system output product state for that system experience. This may be enabled by monitoring any suitable system data for a system. The management model custodian may provide a data collection portal for enabling any suitable entity(ies) to provide such data. The system output state may be received and may be derived from the system in any suitable manner.

A learning engine or comfort model for a system may be using the received monitored system data for the system experience (e.g., as inputs of a neural network of the learning engine) and using the received system output product state for the system experience (e.g., as an output of the neural network of the learning engine). Any suitable training methods or algorithms (e.g., learning algorithms) may be used to train the neural network of the learning engine, including, but not limited to, Back Propagation, Resilient Propagation, Genetic Algorithms, Simulated Annealing, Levenberg, Nelder-Meade, and/or the like. Such training methods may be used individually and/or in different combinations to get the best performance from a neural network. A loop (e.g., a receipt and train loop) of receiving monitored system data and a system output product state for a system experience (e.g., a particular system in a particular environment at a particular moment) and then training the system model using the received monitored system data and system output product state may be repeated any suitable number of times for the same system(s) in different system experiences (e.g., in same or different environments at different moments) and the same learning engine for more effectively training the learning engine for the system, where the received monitored system data and the received system output product state of different receipt and train loops may be for different environments or for the same environment (e.g., at different times and/or with respect to different planned activities) and/or may be received from the same source or from different sources of the system, while the training of different receipt and train loops may be done for the same learning engine using whatever monitored system data and system output product state was received for the particular receipt and train loop. The number and/or type(s) of the one or more monitored system data categories for which monitored system data may be received for one receipt and train loop may be the same or different in any way(s) than the number and/or type(s) of the one or more monitored system data categories for which monitored system data may be received for a second receipt and train loop.

A trained model may then receive input data from any suitable source using any suitable methods for use by the model. The trained model may then use this new input data to generate output data using the learning engine or model. For example, the new input data may be utilized as input(s) to the neural network of the learning engine similarly to how other input data accessed for a receipt and train loop may be utilized as input(s) to the neural network of the learning engine at a training portion of the receipt and train loop, and such utilization of the learning engine with respect to the new input data may result in the neural network providing an output indicative of data that may represent the learning engine's predicted or estimated result.

The processing power and speed of any suitable calibration system and its various models may be configured to determine continuously an updated system output product state of a system and present associated information or otherwise adjust a managed element based on the determined system output product state automatically and instantaneously or substantially instantaneously based on any new received monitored system data that may be generated by the system, such that management of the system may run quickly and smoothly. This may enable the system to operate as effectively and as efficiently as possible.

The use of one or more suitable models or engines or neural networks or the like may enable prediction or any suitable determination of an output product state of a system in a system experience. Such models (e.g., neural networks) running on any suitable processing units (e.g., graphical processing units (“GPUs”) that may be available to the system) provide significant speed improvements in efficiency and accuracy with respect to prediction over other types of algorithms and human-conducted analysis of data, as such models can provide estimates in a few milliseconds or less, thereby improving the functionality of any computing device on which they may be run. Due to such efficiency and accuracy, such models enable a technical solution for enabling the generation of any suitable control data (e.g., for controlling any suitable functionality of any suitable managed element) using any suitable real-time data (e.g., data made available to the models) that may not be possible without the use of such models, as such models may increase performance of their computing device(s) by requiring less memory, providing faster response times, and/or increased accuracy and/or reliability. Due to the condensed time frame and/or the time within which a decision with respect to system data ought to be made to provide a desirable use experience, such models offer the unique ability to provide accurate determinations with the speed necessary to enable effective and efficient use management.

Terms used herein should be accorded their ordinary meaning in the relevant arts, or the meaning indicated by their use in context, but if an express definition is provided, that meaning controls.

“Circuitry” may refer to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment). “Firmware” may refer to software logic embodied as processor-executable instructions stored on volatile memory media and/or non-volatile memory media. “Hardware”, in certain embodiments, may refer to logic embodied as analog and/or digital circuitry. “Software” may refer to logic implemented as processor-executable instructions in a machine memory (e.g. Read/write volatile memory media or non-volatile memory media).

Various functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

Within this disclosure, different entities (which may variously be referred to as “units,” “circuits,” other components, etc.) may be described or claimed as “configured” to perform one or more tasks or operations. This formulation-[entity] configured to [perform one or more tasks]—is used herein to refer to structure (i.e., something physical, such as an electronic circuit). More specifically, this formulation is used to indicate that this structure is arranged to perform the one or more tasks during operation. A structure can be said to be “configured to” perform some task even if the structure is not currently being operated. A “credit distribution circuit configured to distribute credits to a plurality of processor cores” is intended to cover, for example, an integrated circuit that has circuitry that performs this function during operation, even if the integrated circuit in question is not currently being used (e.g., a power supply is not connected to it). Thus, an entity described or recited as “configured to” perform some task may refer to something physical, such as a device, circuit, memory storing program instructions executable to implement the task, etc. This phrase is not used herein to refer to something intangible.

The term “configured to” is not intended to mean “configurable to.” An unprogrammed FPGA, for example, would not be considered to be “configured to” perform some specific function, although it may be “configurable to” perform that function after programming.

“Data management platform” in this context may refer to a centralized system for collecting and analyzing large sets of data originating from disparate sources. A data management platform creates a combined development and delivery environment that provides users with consistent, accurate and timely data. At its simplest, a data management platform could be a database management system that imports data from many systems and enables users to view the data in a consistent manner. A high-end data management platform might combine data management technologies and data analytics tools into a single software suite. A key role of a data management platform is to collect structured and unstructured data from a range of internal and external sources, and to then integrate and store that data. These platforms also analyze and organize data to provide insight to data-driven parts of the business.

As may be used in this specification and any claims of this application, the terms “base station,” “receiver,” “computer,” “server,” “processor,” and “memory” may all refer to electronic or other technological devices. These terms exclude people or groups of people. For the purposes of the specification, the terms “display” or “displaying” means displaying on an electronic device.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. The term “and/or” as used herein may refer to and encompasses any and all possible combinations of one or more of the associated listed items. As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” may each refer to only A, only B, or only C; any combination of A, B, and C; and/or at least one of each of A, B, and C. The terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. When used in the claims, the term “or” is used as an inclusive or and not as an exclusive or. For example, the phrase “at least one of x, y, or z” means any one of x, y, and z, as well as any combination thereof.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

As used herein, the term “or” can be construed in either an inclusive or exclusive sense. Moreover, plural instances can be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and can fall within a scope of various implementations of the present disclosure. In general, structures and functionality presented as separate resources in the example configurations can be implemented as a combined structure or resource. Similarly, structures and functionality presented as a single resource can be implemented as separate resources. These and other variations, modifications, additions, and improvements fall within a scope of implementations of the present disclosure as represented by the appended claims. The specification and drawings are, accordingly, to be regarded in an illustrative rather than a restrictive sense.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As may be used herein, the terms “computer,” “personal computer,” “device,” “computing device,” “router device,” and “controller device” may refer to any programmable computer system that is known or that will be developed in the future. In certain embodiments, a computer will be coupled to a network, such as described herein. A computer system may be configured with processor-executable software instructions to perform the processes described herein. Such computing devices may be mobile devices, such as a mobile telephone, data assistant, tablet computer, or other such mobile device. Alternatively, such computing devices may not be mobile (e.g., in at least certain use cases), such as in the case of server computers, desktop computing systems, or systems integrated with non-mobile components.

As may be used herein, the terms “component,” “module,” and “system,” are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server may be a component. One or more components may reside within a process and/or thread of execution and a component may be localized on one computer and/or distributed between two or more computers.

The predicate words “configured to,” “operable to,” “operative to,” and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation or the processor being operative to monitor and control the operation. Likewise, a processor configured to execute code can be construed as a processor programmed to execute code or operable to execute code or operative to execute code.

As used herein, the term “based on” may be used to describe one or more factors that may affect a determination. However, this term does not exclude the possibility that additional factors may affect the determination. For example, a determination may be solely based on specified factors or based on the specified factors as well as other, unspecified factors. The phrase “determine A based on B” specifies that B is a factor that is used to determine A or that affects the determination of A. However, this phrase does not exclude that the determination of A may also be based on some other factor, such as C. This phrase is also intended to cover an embodiment in which A may be determined based solely on B. As used herein, the phrase “based on” may be synonymous with the phrase “based at least in part on.”

As used herein, the phrase “in response to” may be used to describe one or more factors that trigger an effect. This phrase does not exclude the possibility that additional factors may affect or otherwise trigger the effect. For example, an effect may be solely in response to those factors, or may be in response to the specified factors as well as other, unspecified factors. The phrase “perform A in response to B” specifies that B is a factor that triggers the performance of A. However, this phrase does not foreclose that performing A may also be in response to some other factor, such as C. This phrase is also intended to cover an embodiment in which A is performed solely in response to B.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”. Reciting in the appended claims that a structure is “configured to” perform one or more tasks is expressly intended not to invoke 35 U.S.C. § 112(f) for that claim element. Accordingly, claims in this application that do not otherwise include the “means for” [performing a function] construct should not be interpreted under 35 U.S.C § 112(f).

As used herein, the terms “first,” “second,” etc. Are used as labels for nouns that they precede, and do not imply any type of ordering (e.g., spatial, temporal, logical, etc.), unless stated otherwise. For example, in a register file having eight registers, the terms “first register” and “second register” can be used to refer to any two of the eight registers, and not, for example, just logical registers 0 and 1.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” may refer to one or more. Pronouns in the masculine (e.g., his) include the feminine and neuter/neutral gender (e.g., her and its and they) and vice versa. Headings and subheadings, if any, are used for convenience only and do not limit the subject disclosure.

While there have been described systems, methods, and computer-readable media for calibrating air quality sensors, many changes may be made therein without departing from the spirit and scope of the subject matter described herein in any way. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. It is also to be understood that various directional and orientational terms, such as “left” and “right,” “up” and “down,” “front” and “back” and “rear,” “top” and “bottom” and “side,” “above” and “below,” “length” and “width” and “thickness” and “diameter” and “cross-section” and “longitudinal,” “X-” and “Y-” and “Z-,” “roll” and “pitch” and “yaw,” “clockwise” and “counter-clockwise,” and/or the like, may be used herein only for convenience, and that no fixed or absolute directional or orientational limitations are intended by the use of these terms. For example, the components of the apparatus can have any desired orientation. If reoriented, different directional or orientational terms may need to be used in their description, but that will not alter their fundamental nature as within the scope and spirit of the disclosure.

It is to be understood that the disclosed subject matter is not limited in its application to the details of construction and to the arrangements of the components set forth in the above description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, systems, methods, and media for carrying out the several purposes of the disclosed subject matter. It is important, therefore, that the claims be regarded as including such equivalent constructions insofar as they do not depart from the spirit and scope of the disclosed subject matter. Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims which follow. Having thus described illustrative embodiments in detail, it will be apparent that modifications and variations are possible without departing from the scope of the disclosure as claimed. The scope of patentable subject matter is not limited to the depicted embodiments but is rather set forth in the following Claims.

Therefore, those skilled in the art will appreciate that the concepts of the disclosure can be practiced by other than the described embodiments, which are presented for purposes of illustration rather than of limitation.

What is claimed is:

1. A method comprising:
accessing collocation data for each one of a plurality of collocations, wherein:
the accessed collocation data for each collocation of the plurality of collocations comprises:
sensor data collected from a sensor of the collocation over a collocation period of time of the collocation; and
reference monitor data collected from a reference monitor of the collocation over the collocation period of time of the collocation; and
for each collocation of the plurality of collocations, the sensor of the collocation was collocated with the reference monitor of the collocation during the collocation period of time of the collocation at a location of the collocation;
combining the accessed collocation data from each one the plurality of collocations into global collocation data;
developing a global calibration on the global collocation data; and
configuring a calibration regulator for a sensor of interest (“SOI”) to apply the developed global calibration.

2. The method of claim 1, wherein the SOI is not a sensor of any collocation of the plurality of collocations.

3. The method of claim 1, wherein the sensor of each collocation of the plurality of collocations is the same particular type of sensor.

4. The method of claim 1, wherein the location of a first collocation of the plurality of collocations is different than the location of a second collocation of the plurality of collocations.

5. The method of claim 1, wherein the collocation period of time of a first collocation of the plurality of collocations is different than the collocation period of time of a second collocation of the plurality of collocations.

6. The method of claim 1, wherein:
the location of a first collocation of the plurality of collocations is different than the location of a second collocation of the plurality of collocations; and
the collocation period of time of a first collocation of the plurality of collocations is different than the collocation period of time of a second collocation of the plurality of collocations.

7. The method of claim 1, further comprising:
positioning the SOI in an SOI monitoring position;
after the positioning, obtaining an SOI sensor reading from the SOI; and
correcting the obtained SOI sensor reading with the developed global calibration using the configured calibration regulator for the SOI.

8. The method of claim 7, wherein:
the positioning comprises positioning the SOI in the SOI monitoring position to be collocated with an SOI reference monitor;
the obtaining comprises obtaining the SOI sensor reading from the SOI and obtaining a reference measurement from the SOI reference monitor; and
the method further comprises performing a linear regression between the corrected SOI sensor reading and the obtained reference measurement.

9. The method of claim 8, further comprising developing a global calibration scaling based on the performed linear regression.

10. The method of claim 9, further comprising:
moving the SOI to a different SOI monitoring position after the obtaining;
after the moving, obtaining another SOI sensor reading from the SOI; and
correcting the obtained other SOI sensor reading with the developed global calibration and with the developed global calibration scaling.

11. The method of claim 9, further comprising further configuring the calibration regulator for the SOI to apply the developed global calibration scaling.

12. The method of claim 11, further comprising:
moving the SOI to a different SOI monitoring position after the obtaining;
after the moving, obtaining another SOI sensor reading from the SOI; and
correcting the obtained other SOI sensor reading with the developed global calibration and with the developed global calibration scaling using the further configured calibration regulator for the SOI.

13. The method of claim 1, further comprising, prior to the configuring, providing a plurality of SOIs that comprises the SOI and at least one other SOI, wherein the configuring comprises configuring a calibration regulator for each SOI of the plurality of SOIs to apply the developed global calibration.

14. The method of claim 13, further comprising:
positioning the SOI in an SOI monitoring position that is collocated with an SOI reference monitor;
after the positioning, obtaining an SOI sensor reading from the SOI and obtaining a reference measurement from the SOI reference monitor;
correcting the obtained SOI sensor reading with the developed global calibration using the configured calibration regulator for the SOI; and
performing a linear regression between the corrected SOI sensor reading and the obtained reference measurement.

15. The method of claim 14, further comprising developing a global calibration scaling based on the performed linear regression.

16. The method of claim 15, further comprising:
placing the at least one other SOI in a different SOI monitoring position than the SOI monitoring position;
after the placing, obtaining another SOI sensor reading from the at least one other SOI; and
correcting the obtained other SOI sensor reading with the developed global calibration and with the developed global calibration scaling.

17. The method of claim 15, further comprising further configuring the calibration regulator for the at least one other SOI to apply the developed global calibration scaling.

18. The method of claim 17, further comprising:
placing the at least one other SOI in a different SOI monitoring position than the SOI monitoring position;
after the placing, obtaining another SOI sensor reading from the at least one other SOI; and
correcting the obtained other SOI sensor reading with the developed global calibration and with the developed global calibration scaling using the further configured calibration regulator for the at least one other SOI.

19. A non-transitory computer-readable storage medium storing at least one program, the at least one program comprising instructions, which, when executed by at least one processor of an electronic subsystem, cause the at least one processor to:
access collocation data for each one of a plurality of collocations, wherein:
the accessed collocation data for each collocation of the plurality of collocations comprises:
sensor data collected from a sensor of the collocation over a collocation period of time of the collocation; and
reference monitor data collected from a reference monitor of the collocation over the collocation period of time of the collocation; and
for each collocation of the plurality of collocations, the sensor of the collocation was collocated with the reference monitor of the collocation during the collocation period of time of the collocation at a location of the collocation;
combine the accessed collocation data from each one the plurality of collocations into global collocation data;
develop a global calibration on the global collocation data; and
configure a calibration regulator for a sensor of interest (“SOI”) to apply the developed global calibration.

20. A system comprising:
a memory component;
a communications component; and
a processor component configured to:
access collocation data for each one of a plurality of collocations using the communications component, wherein:
the accessed collocation data for each collocation of the plurality of collocations comprises:
sensor data collected from a sensor of the collocation over a collocation period of time of the collocation; and
reference monitor data collected from a reference monitor of the collocation over the collocation period of time of the collocation; and
for each collocation of the plurality of collocations, the sensor of the collocation was collocated with the reference monitor of the collocation during the collocation period of time of the collocation at a location of the collocation;
combine the accessed collocation data from each one the plurality of collocations into global collocation data;
develop a global calibration on the global collocation data; and
configure, in the memory component, a calibration regulator for a sensor of interest (“SOI”) to apply the developed global calibration.
